SEQUENCE LISTING (145 sequences)

SEQ ID NO: 1 - protein, 133 aa, Homo sapiens [sequence omitted]

SEQ ID NO: 2 - protein, 132 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTL

SEQ ID NO: 3 - protein, 131 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIIST

SEQ ID NO: 4 - protein, 130 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIIS

SEQ ID NO: 5 - protein, 129 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSII

SEQ ID NO: 6 - protein, 128 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSI

SEQ ID NO: 7 - protein, 127 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQS

SEQ ID NO: 8 - protein, 126 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQ

SEQ ID NO: 9 - protein, 125 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFC

SEQ ID NO: 10 - protein, 124 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITF

SEQ ID NO: 11 - protein, 133 aa, synthetic construct
APTSSSTKKTQLQLEHLLLDLQMILNGINNYKNPKLTRMLTGKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 12 - protein, 133 aa, synthetic construct
APTSSSTKKTQLQLEHLLLDLQMILNGINNYKNPKLTRMLTVKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 13 - protein, 133 aa, synthetic construct
APTSSSTKKTQLQLEHLLLDLQMILNGINNYKNPKLTRMLTIKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 14 - protein, 129 aa, synthetic construct
APTSSSTKKTQLQLEHLLLDLQMILNGINNYKNPKLTRMLTVKFYMPKKATELKHLQCLEEELKPLEEVLNLAQSKNFHLRPRDLISNINVIVLELKGSETTFMCEYADETATIVEFLNRWITFCQSII

SEQ ID NO: 15 - protein, 129 aa, synthetic construct
APTSSSTKKTQLQLEHLLLDLQMILNGINNYKNPKLTRMLTIKFYMPKKATELKHLQCLEEELKPLEEVLNLAQSKNFHLRPRDLISNINVIVLELKGSETTFMCEYADETATIVEFLNRWITFCQSII

SEQ ID NO: 16 - protein, 128 aa, synthetic construct
APTSSSTKKTQLQLEHLLLDLQMILNGINNYKNPKLTRMLTVKFYMPKKATELKHLQCLEEELKPLEEVLNLAQSKNFHLRPRDLISNINVIVLELKGSETTFMCEYADETATIVEFLNRWITFCQSI

SEQ ID NO: 17 - protein, 128 aa, synthetic construct
APTSSSTKKTQLQLEHLLLDLQMILNGINNYKNPKLTRMLTIKFYMPKKATELKHLQCLEEELKPLEEVLNLAQSKNFHLRPRDLISNINVIVLELKGSETTFMCEYADETATIVEFLNRWITFCQSI

SEQ ID NO: 18 - protein, 129 aa, synthetic construct
APTSSSTKKTQLQLEHLLLDLQMILNGINNYKNPKLTRMLTMKFYMPKKATELKHLQCLEEELKPLEEVLNLAQSKNFHLRPRDLISNINVIVLELKGSETTFMCEYADETATIVEFLNRWITFCQSII

SEQ ID NO: 19 - protein, 129 aa, synthetic construct
APTSSSTKKTQLQLEHLLLDLQMILNGINNYKNPKLTWMLTFKFYMPKKATELKHLQCLEEELKPLEEVLNLAQSKNFHLRPRDLISNINVIVLELKGSETTFMCEYADETATIVEFLNRWITFCQSII

SEQ ID NO: 20 - protein, 129 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADEAATIVEFLNRWITFCQSII

SEQ ID NO: 21 - protein, 129 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADEEATIVEFLNRWITFCQSII

SEQ ID NO: 22 - protein, 133 aa, synthetic construct
APTSSSTKKTQLQLEHLLLDLQMILNGINNYKNPKLTWMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 23 - protein, 129 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIL

SEQ ID NO: 24 - protein, 129 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIV

SEQ ID NO: 25 - protein, 129 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIA

SEQ ID NO: 26 - protein, 129 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIS

SEQ ID NO: 27 - protein, 129 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIT

SEQ ID NO: 28 - protein, 129 aa, synthetic construct
APTSSSTKKTQLQLEHLLLDLQMILNGINNYKNPKLTRMLTVKFYMPKKATELKHLQCLEEELKPLEEVLNLAQSKNFHLRPRDLISNINVIVLELKGSETTFMCEYADETATIVEFLNRWITFCQSIL

SEQ ID NO: 29 - protein, 129 aa, synthetic construct
APTSSSTKKTQLQLEHLLLDLQMILNGINNYKNPKLTRMLTIKFYMPKKATELKHLQCLEEELKPLEEVLNLAQSKNFHLRPRDLISNINVIVLELKGSETTFMCEYADETATIVEFLNRWITFCQSIL

SEQ ID NO: 30 - protein, 129 aa, synthetic construct
APTSSSTKKTQLQLEHLLLDLQMILNGINNYKNPKLTWMLTFKFYMPKKATELKHLQCLEEELKPLEEVLNLAQSKNFHLRPRDLISNINVIVLELKGSETTFMCEYADETATIVEFLNRWITFCQSIL

SEQ ID NO: 31 - protein, 129 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQAII

SEQ ID NO: 32 - protein, 129 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQTII

SEQ ID NO: 33 - protein, 129 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQDII

SEQ ID NO: 34 - protein, 129 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQNIS

SEQ ID NO: 35 - protein, 129 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSII

SEQ ID NO: 36 - protein, 129 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSAI

SEQ ID NO: 37 - protein, 129 aa, synthetic construct; identical to SEQ ID NO: 5

SEQ ID NO: 38 - protein, 129 aa, synthetic construct; identical to SEQ ID NO: 5

SEQ ID NO: 39 - protein, 129 aa, synthetic construct; identical to SEQ ID NO: 5

SEQ ID NO: 40 - protein, 129 aa, synthetic construct; identical to SEQ ID NO: 5

SEQ ID NO: 41 - protein, 129 aa, synthetic construct; identical to SEQ ID NO: 5

SEQ ID NO: 42 - protein, 129 aa, synthetic construct; identical to SEQ ID NO: 5

SEQ ID NO: 43 - protein, 129 aa, synthetic construct; identical to SEQ ID NO: 5

SEQ ID NO: 44 - protein, 129 aa, synthetic construct; identical to SEQ ID NO: 5

SEQ ID NO: 45 - protein, 129 aa, synthetic construct; identical to SEQ ID NO: 5

SEQ ID NO: 46 - protein, 129 aa, synthetic construct; identical to SEQ ID NO: 5

SEQ ID NO: 47 - protein, 128 aa, synthetic construct; identical to SEQ ID NO: 6

SEQ ID NO: 48 - protein, 129 aa, synthetic construct; identical to SEQ ID NO: 5

SEQ ID NO: 49 - protein, 129 aa, synthetic construct; identical to SEQ ID NO: 23

SEQ ID NO: 50 - protein, 129 aa, synthetic construct; identical to SEQ ID NO: 23

SEQ ID NO: 51 - protein, 129 aa, synthetic construct; identical to SEQ ID NO: 23

SEQ ID NO: 52 - protein, 129 aa, synthetic construct; identical to SEQ ID NO: 23

SEQ ID NO: 53 - protein, 129 aa, synthetic construct; identical to SEQ ID NO: 23

SEQ ID NO: 54 - protein, 129 aa, synthetic construct; identical to SEQ ID NO: 23

SEQ ID NO: 55 - protein, 129 aa, synthetic construct; identical to SEQ ID NO: 23

SEQ ID NO: 56 - protein, 129 aa, synthetic construct; identical to SEQ ID NO: 23

SEQ ID NO: 57 - protein, 129 aa, synthetic construct; identical to SEQ ID NO: 23

SEQ ID NO: 58 - protein, 129 aa, synthetic construct; identical to SEQ ID NO: 23

SEQ ID NO: 59 - protein, 129 aa, synthetic construct; identical to SEQ ID NO: 23

SEQ ID NO: 60 - protein, 129 aa, synthetic construct; identical to SEQ ID NO: 23

SEQ ID NO: 61 - protein, 133 aa, synthetic construct
APTSSSTKKTQLQLEHLLLDLQMILNGINNYKNPKLTRMLTVKFAMPKKATELKHLQCLEEELKPLEEVLNGAQSKNFHLRPRDLISNINVIVLELKGSETTFMCEYADETATIVEFLNRWITFCQSIISTLT

SEQ ID NO: 62 - protein, 597 aa, synthetic construct
EIQLQQSGPELVKPGASVKVSCKASGYSFTDYNMYWVKQSHGESLEWIGYIDPYNGGTRYNQKFKGKATLTVDKSSSTAFMHLNSLTSEDSAVYYCARNGHWDGAWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 63 - protein, 597 aa, synthetic construct
EIQLQQSGPELVKPGASVKVSCKASGYSFTDYNMYWVKQSHGESLEWIGYIDPYNGGTRYNQKFKGKATLTVDKSSSTAFMHLNSLTSEDSAVYYCARNGHWDGAWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFCQSIISTLT

SEQ ID NO: 64 - protein, 361 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 65 - protein, 379 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 66 - protein, 361 aa, synthetic construct
APTSSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFCQSIISTLTESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 67 - protein, 379 aa, synthetic construct
APTSSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFCQSIISTLTGGGGSGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 68 - protein, 360 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 69 - protein, 359 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 70 - protein, 358 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 71 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIIESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 72 - protein, 356 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 73 - protein, 355 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 74 - protein, 354 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 75 - protein, 353 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 76 - protein, 352 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 77 - protein, 361 aa, synthetic construct
APTSSSTKKTQLQLEHLLLDLQMILNGINNYKNPKLTRMLTGKFYMPKKATELKHLQCLEEELKPLEEVLNLAQSKNFHLRPRDLISNINVIVLELKGSETTFMCEYADETATIVEFLNRWITFCQSIISTLTESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 78 - protein, 361 aa, synthetic construct
APTSSSTKKTQLQLEHLLLDLQMILNGINNYKNPKLTRMLTVKFYMPKKATELKHLQCLEEELKPLEEVLNLAQSKNFHLRPRDLISNINVIVLELKGSETTFMCEYADETATIVEFLNRWITFCQSIISTLTESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 79 - protein, 361 aa, synthetic construct
APTSSSTKKTQLQLEHLLLDLQMILNGINNYKNPKLTRMLTIKFYMPKKATELKHLQCLEEELKPLEEVLNLAQSKNFHLRPRDLISNINVIVLELKGSETTFMCEYADETATIVEFLNRWITFCQSIISTLTESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 80 - protein, 357 aa, synthetic construct
APTSSSTKKTQLQLEHLLLDLQMILNGINNYKNPKLTRMLTVKFYMPKKATELKHLQCLEEELKPLEEVLNLAQSKNFHLRPRDLISNINVIVLELKGSETTFMCEYADETATIVEFLNRWITFCQSIIESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 81 - protein, 357 aa, synthetic construct
APTSSSTKKTQLQLEHLLLDLQMILNGINNYKNPKLTRMLTIKFYMPKKATELKHLQCLEEELKPLEEVLNLAQSKNFHLRPRDLISNINVIVLELKGSETTFMCEYADETATIVEFLNRWITFCQSIIESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 82 - protein, 356 aa, synthetic construct
APTSSSTKKTQLQLEHLLLDLQMILNGINNYKNPKLTRMLTVKFYMPKKATELKHLQCLEEELKPLEEVLNLAQSKNFHLRPRDLISNINVIVLELKGSETTFMCEYADETATIVEFLNRWITFCQSIESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 83 - protein, 356 aa, synthetic construct
APTSSSTKKTQLQLEHLLLDLQMILNGINNYKNPKLTRMLTIKFYMPKKATELKHLQCLEEELKPLEEVLNLAQSKNFHLRPRDLISNINVIVLELKGSETTFMCEYADETATIVEFLNRWITFCQSIESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 84 - protein, 357 aa, synthetic construct
APTSSSTKKTQLQLEHLLLDLQMILNGINNYKNPKLTRMLTMKFYMPKKATELKHLQCLEEELKPLEEVLNLAQSKNFHLRPRDLISNINVIVLELKGSETTFMCEYADETATIVEFLNRWITFCQSIIESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 85 - protein, 357 aa, synthetic construct
APTSSSTKKTQLQLEHLLLDLQMILNGINNYKNPKLTWMLTFKFYMPKKATELKHLQCLEEELKPLEEVLNLAQSKNFHLRPRDLISNINVIVLELKGSETTFMCEYADETATIVEFLNRWITFCQSIIESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 86 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADEAATIVEFLNRWITFCQSIIESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 87 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADEEATIVEFLNRWITFCQSIIESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 88 - protein, 361 aa, synthetic construct
APTSSSTKKTQLQLEHLLLDLQMILNGINNYKNPKLTWMLTFKFYMPKKATELKHLQCLEEELKPLEEVLNLAQSKNFHLRPRDLISNINVIVLELKGSETTFMCEYADETATIVEFLNRWITFCQSIISTLTESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 89 - protein, 356 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 90 - protein, 355 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIIKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 91 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSILESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 92 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 93 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIAESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 94 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSISESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 95 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSITESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 96 - protein, 357 aa, synthetic construct
APTSSSTKKTQLQLEHLLLDLQMILNGINNYKNPKLTRMLTVKFYMPKKATELKHLQCLEEELKPLEEVLNLAQSKNFHLRPRDLISNINVIVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 97 - protein, 357 aa, synthetic construct
APTSSSTKKTQLQLEHLLLDLQMILNGINNYKNPKLTRMLTIKFYMPKKATELKHLQCLEEELKPLEEVLNLAQSKNFHLRPRDLISNINVIVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 98 - protein, 357 aa, synthetic construct
APTSSSTKKTQLQLEHLLLDLQMILNGINNYKNPKLTWMLTFKFYMPKKATELKHLQCLEEELKPLEEVLNLAQSKNFHLRPRDLISNINVIVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 99 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQAIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 100 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQTIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 101 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQDIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 102 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQNISESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 103 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 104 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSAIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 105 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADRTATIVEFLNRWITFCQSIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 106 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADITATIVEFLNRWITFCQSIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 107 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADTTATIVEFLNRWITFCQSIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 108 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWYTFCQSIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 109 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWTTFCQSIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 110 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWVTFCQSIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 111 - protein, 357 aa, synthetic construct
APTSSSTKKTQLQLEHLLLDLQMILNGPNNYKNPKLTRMLTFKFYMPKKATELKHLQCLEEELKPLEEVLNLAQSKNFHLRPRDLISNINVIVLELKGSETTFMCEYADETATIVEFLNRWITFCQSIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 112 - protein, 357 aa, synthetic construct
APTSSSTKKTQLQLEHLLLDLQMILNGINNYDNPKLTRMLTFKFYMPKKATELKHLQCLEEELKPLEEVLNLAQSKNFHLRPRDLISNINVIVLELKGSETTFMCEYADETATIVEFLNRWITFCQSIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 113 - protein, 357 aa, synthetic construct
APTSSSTKKTQLQLEHLLLDLQMILNGINNYKNPKLTRMLTFKFYMPKKATGLKHLQCLEEELKPLEEVLNLAQSKNFHLRPRDLISNINVIVLELKGSETTFMCEYADETATIVEFLNRWITFCQSIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 114 - protein, 357 aa, synthetic construct
APTSSSTKKTQLQLEHLLLDLQMILNGINNYKNPKLTRMLTFKFYMPKKATELKHLQCLEEELKPLEEVLNLAQSRNFHLRPRDLISNINVIVLELKGSETTFMCEYADETATIVEFLNRWITFCQSIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 115 - protein, 357 aa, synthetic construct
APTSSSTKKTQLQLEHLLLDLQMILNGINNYKNPKLTRMLTFKFYMPKKATELKHLQCLEEELKPLEEVLNLAQSKNGHLRPRDLISNINVIVLELKGSETTFMCEYADETATIVEFLNRWITFCQSIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 116 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRYRDLISNINVIVLELKGSETTFMCEYADETATIVEFLNRWITFCQSIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 117 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADRTATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 118 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADITATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 119 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADTTATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 120 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWYTFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 121 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWTTFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 122 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWVTFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 123 - protein, 357 aa, synthetic construct
APTSSSTKKTQLQLEHLLLDLQMILNGPNNYKNPKLTRMLTFKFYMPKKATELKHLQCLEEELKPLEEVLNLAQSKNFHLRPRDLISNINVIVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 124 - protein, 357 aa, synthetic construct
APTSSSTKKTQLQLEHLLLDLQMILNGINNYDNPKLTRMLTFKFYMPKKATELKHLQCLEEELKPLEEVLNLAQSKNFHLRPRDLISNINVIVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 125 - protein, 357 aa, synthetic construct
APTSSSTKKTQLQLEHLLLDLQMILNGINNYKNPKLTRMLTFKFYMPKKATGLKHLQCLEEELKPLEEVLNLAQSKNFHLRPRDLISNINVIVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 126 - protein, 357 aa, synthetic construct
APTSSSTKKTQLQLEHLLLDLQMILNGINNYKNPKLTRMLTFKFYMPKKATELKHLQCLEEELKPLEEVLNLAQSRNFHLRPRDLISNINVIVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 127 - protein, 357 aa, synthetic construct
APTSSSTKKTQLQLEHLLLDLQMILNGINNYKNPKLTRMLTFKFYMPKKATELKHLQCLEEELKPLEEVLNLAQSKNGHLRPRDLISNINVIVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 128 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRYRDLISNINVIVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 129 - protein, 361 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 130 - protein, 361 aa, synthetic construct
APTSSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFCQSIISTLTESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 131 - protein, 449 aa, synthetic construct
EIQLQQSGPELVKPGASVKVSCKASGYSFTDYNMYWVKQSHGESLEWIGYIDPYNGGTRYNQKFKGKATLTVDKSSSTAFMHLNSLTSEDSAVYYCARNGHWDGAWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 132 - protein, 446 aa, synthetic construct
EIQLQQSGPELVKPGASVKVSCKASGYSFTDYNMYWVKQSHGESLEWIGYIDPYNGGTRYNQKFKGKATLTVDKSSSTAFMHLNSLTSEDSAVYYCARNGHWDGAWFAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 133 - protein, 446 aa, synthetic construct
EIQLQQSGPELVKPGASVKVSCKASGYSFTDYNMYWVKQSHGESLEWIGYIDPYNGGTRYNQKFKGKATLTVDKSSSTAFMHLNSLTSEDSAVYYCARNGHWDGAWFAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 134 - protein, 214 aa, synthetic construct
DIQMNQSPSSLSASLGDTITITCHASQNINVWLSWYQQKPGNIPKLLIYKASNLHTGVPSRFSGSRSGTGFTLTISSLQPEDIATYYCHQGQSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - protein, 198 aa, synthetic construct
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCHHHHHH

SEQ ID NO: 136 - protein, 230 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDLEGGGGSGGGGHHHHHH

SEQ ID NO: 137 - protein, 249 aa, synthetic construct
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENLEGGGGSGGGGHHHHHH

SEQ ID NO: 138 - protein, 358 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 139 - protein, 358 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 140 - protein, 361 aa, synthetic construct
APASSSTKKTQLQLEHLLLNLQMILNGINNYKNPKLTRMLTFKFYMPKKATELKHLQCLEEELKPLEEVLKLAQSKNFHLRPRDLISNINVIVLELKGSETTFMCEYADETATIVEFLNRWITFSQSIISTLTESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 141 - protein, 370 aa, synthetic construct
APTSSSTSSSTAEAQQQQQHLEQLRMDLEELLSRMENYRNLKLPRMLTFKFYLPKQATELKDLOCLEDELGPLRHVLDLTOSKSFQLEDAENFISNIRVTVVKLKGSDNTFECOFDDESATVVDFLRRWIAFCHSIISTSPOESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 142 - protein, 361 aa, synthetic construct
APTSSSTKKTQLQLEHLRLDLEMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCKSIISTLTESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 143 - protein, 361 aa, synthetic construct
APTSSSTKKTQLQLEHLLHDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIESIISTLTESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 144 - protein, 498 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSEIQLQQSGPELVKPGASVKVSCKASGYSFTDYNMYWVKQSHGESLEWIGYIDPYNGGTRYNQKFKGKATLTVDKSSSTAFMHLNSLTSEDSAVYYCARNGHWDGAWFAYWGQGTLVTVSA

SEQ ID NO: 145 - protein, 498 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSEIQLQQSGPELVKPGASVKVSCKASGYSFTDYNMYWVKQSHGESLEWIGYIDPYNGGTRYNQKFKGKATLTVDKSSSTAFMHLNSLTSEDSAVYYCARNGHWDGAWFAYWGQGTLVTVSA

(19) United States
(12) Patent Application Publication
(10) Pub. No.: US 2025/0000946 A1
(43) Pub. Date: Jan. 2, 2025

(54) IL-2 VARIANTS AND FUSION PROTEINS THEREOF

(71) Applicant: WUXI BIOLOGICS IRELAND LIMITED, CO LOUTH (IE)

(72) Inventors: Lei WU, SHANGHAI (CN); Jianqing XU, SHANGHAI (CN); Mengmeng SUN, SHANGHAI (CN); Shuang WANG, SHANGHAI (CN); Jijie GU, SHANGHAI (CN)

(21) Appl. No.: 18/694,718

(22) PCT Filed: Sep. 26, 2022
(86) PCT No.: PCT/CN2022/121283

§ 371 Date: Mar. 22, 2024

(30) Foreign Application Priority Data
Sep. 26, 2021 (WO) PCT/CN2021/120702

(51) Int. Cl. A61K 38/20; C07K 14/55

(52) U.S. Cl. CPC A61K 38/2013; C07K 14/55; C07K 2319/30

(57) ABSTRACT

Provided are IL-2 variants and polypeptide complexes comprising the IL-2 variants, the methods of producing the same and the uses thereof. The IL-2 variants and fusion proteins can be used as a potent agent for the treatment of cancers, autoimmune and inflammatory diseases.

### CROSS-REFERENCES

This application claims benefit of priority of International Patent Application No. PCT/CN2021/120702 filed on Sep. 26, 2021, the disclosure of which is incorporated by reference herein in its entirety.

### SEQUENCE LISTING

The instant application contains a sequence listing which is hereby incorporated by reference in its entirety.

### FIELD

This application generally relates to IL-2 variants, fusion proteins and polypeptide complexes comprising the IL-2 variant, a method for preparing the same, and the uses thereof.

### BACKGROUND

Interleukin-2 (IL-2) is a four α-helical bundle cytokine identified as a growth factor of T cells, involving in the growth and proliferation of many immune cells [Morgan D A, Ruscetti F W, and Gallo R. Selective in vitro growth of T lymphocytes from normal human bone marrows. Science 1976; 193: 1007-1008]. IL-2 is mainly produced by activated T cells. The biological activity of IL-2 is mediated through three transmembrane receptor subunits: IL-2Rα (CD25), IL-2Rβ (CD122) and common γ chain (CD132). NK cells and steady state T cells express IL-2Rβ and γ, and only regulatory T cells (Treg) and activated T cells express IL-2Rα in addition to IL-2Rβ and γ. IL-2Rα serves to facilitate the delivery of IL-2 to the other 2 subunits, and IL-2Rβ/γ transduce activating signals through STAT5-related pathway. The IL-2Rα/β/γ trimer on Treg and activated T cells works as the high affinity receptor (K_{D}˜10⁻¹¹M) of IL-2, and IL-2Rβ/γ (on NK cells and steady state T cells) is the intermediate affinity receptor (K_{D}˜10⁻⁹ M) [Takeshita T, Asao H, Ohtani K, et al. Cloning of the gamma chain of the human IL-2 receptor. Science 1992; 257: 379-382]. The high affinity receptor is critical to capture IL-2 in lower concentration, and make Treg and activated T cells more sensitive to IL-2 to support their respective functions.

IL-2 and its variants has been tested in various clinical trials. IL-2 has shown promising efficacy in cancer immunotherapy, but its toxicity profile has limited its efficacy and use. Recombinant IL-2 showed short half-life and severe side effects such as vascular leak syndrome (VLS). IL-2 variants have been developed to mitigate such problems, however, they have encountered issues including mitigated efficacy or elevated immunogenicity.

Cancer immunotherapy using immuno-checkpoint inhibitors such as anti-PD(L)1 antibodies and other immune modulatory drugs have achieved breakthrough in recent years. However, a significant proportion of cancer patients still remain resistant or refractory to existing cancer immunotherapies. IL-2, as a T and NK cells modulatory cytokine, has the potential to augment responses induced by existing therapies, like therapeutic antibodies to checkpoints (PD1, PD-L1, CTLA4, LAG-3, etc.) or tumor association antigens (Rituximab, Trustuzumab, Daratumumab, etc).

Treg cells play a critical role to maintain immune homeostasis and self-tolerance, and is crucial in controlling the development of allergies and autoimmune diseases. IL-2 is able to induce the proliferation of Treg cells by signaling through high affinity IL-2 receptor. Therefore, expanding Treg population with IL-2 therapy has the potential to balance and limit pathogenic T cells in inflammatory diseases. At present, IL-2 therapy is being tested in clinical trials for inflammatory diseases including graft-versus-host disease, type 1 diabetes and systemic lupus erythematosus [Ye C, Brand D, and Zheng S. G. Targeting IL-2: an unexpected effect in treating immunological diseases. Sig Transduct Target Ther 2018]. Nevertheless, existing IL-2 molecules are still burdened by fast clearance and potential toxicity.

Therapeutic IL-2, therefore, still need further exploration and optimization to generate ideal drug molecules with powerful efficacy and limited toxicity. IL-2 based therapies may meet the huge medical needs in immuno-oncology and autoimmune disease therapeutic areas.

The present disclosure provides potency reduced IL-2 variants and IL-2 fusion proteins thereof, which may serve as a novel immunotherapy agent with improved therapeutic efficacy.

### SUMMARY

These and other objectives are provided for by the present disclosure which, in a broad sense, is directed to compounds, methods, compositions and articles of manufacture that provide proteins with improved efficacy. The benefits provided by the present disclosure are broadly applicable in the field of therapeutics and diagnostics and may be used in conjunction with antibodies that react with a variety of targets.

The present invention is directed to human IL-2 variants, fusion proteins and polypeptide complexes comprising the IL-2 variants. The IL-2 is herein engineered with a unique approach to produce variants—with attenuated affinity to IL-2Rβ/γc, to IL-2Rα, or to both and the combined IL-2Rα/β/γc complex. Though attenuated, the IL-2 variant still retains binding capacity to IL-2Rα, to IL-2Rβ/γc, and to the combined IL-2Rα/β/γc complex. The IL-2 variant has higher affinity to IL-2Rα/β/γc complex than to IL-2Rα or IL-2Rβ/γc.

The IL-2 variants showed different potency and toxicity. The IL-2 variants hence may serve as a novel immunotherapy agent for cancer and autoimmune disease, as a standalone therapy or in combination with other treatment options. In addition, the IL-2 variants may serve as an immunomodulatory agent for autoimmunity and other inflammatory diseases.

In some aspects, the present disclosure provides an IL-2 variant, wherein the IL-2 variant has a modified or reduced binding affinity to at least one of IL-2Rα, IL-2Rβ, and common γ chain, and has an amino acid sequence comprising one or more mutation(s) selected from a C-terminal truncation, a substitution at position 28, a substitution at position 32, a substitution at position 38, a substitution at position 42, a substitution at position 52, a substitution at position 72, a substitution at position 76, a substitution at position 78, a substitution at position 82, a substitution at position 110, a substitution at position 111, a substitution at position 122, a substitution at position 125, a substitution at position 127, a substitution at position 128, and a substitution at position 129 and any combination thereof, compared to the amino acid sequence as set forth in SEQ ID NO: 1.

In some embodiments, the C-terminal truncation is selected from a truncation of 1 to 20 amino acids, such as a truncation of 20, 19, 18, 17, 16, 15, 14, 13, 12, 11, 10, 9, 8, 7, 6, 5, 4, 3, 2 or 1 amino acid(s).

In some embodiments, the substitution at position 28 is selected from a group consisting of: I28A, I28E, I28G, I28V, I28F, I28M, I28W, I28P, I28Q, I28K, I28S, I28T, I28N, I28D, I28R, I28H, I28L and I128Y.

In some embodiments, the substitution at position 32 is selected from a group consisting of: K32A, K32E, K32G, K32V, K32F, K32M, K32W, K32P, K32Q, K32I, K32S, K32T, K32N, K32D, K32R, K32H, K32L and K32Y.

In some embodiments, the substitution at position 38 is selected from a group consisting of: R38W, R38F, R38Y, R38A, R38V, R38G, R38S, R38T, R38L, R38I, R38H, R38K, R38Q, R38N, R38D, R39E, R38M and R38P.

In some embodiments, the substitution at position 42 is selected from a group consisting of: F42G, F42V, F42I, F42M, F42W, F42P, F42Q, F42K, F42A, F42S, F42T, F42E, F42N, F42D, F42R, F42H, F42L and F42Y.

In some embodiments, the substitution at position 52 is selected from a group consisting of: E52A, E52K, E52G, E52V, E52F, E52M, E52W, E52P, E52Q, E52I, E52S, E52T, E52N, E52D, E52R, E52H, E52L and E52Y.

In some embodiments, the substitution at position 76 is selected from a group consisting of: K76A, K76E, K76G, K76V, K76F, K76M, K76W, K76P, K76Q, K76I, K76S, K76T, K76N, K76D, K76R, K76H, K76L and K76Y.

In some embodiments, the substitution at position 78 is selected from a group consisting of: F78A, F78K, F78G, F78V, F78E, F78M, F78W, F78P, F78Q, F78I, F78S, F78T, F78N, F78D, F78R, F78H, F78L and F78Y.

In some embodiments, the substitution at position 82 is selected from a group consisting of: P82A, P82E, P82G, P82V, P82F, P82M, P82W, P82K, P82Q, P82I, P82S, P82T, P82N, P82D, P82R, P82H, P82L and P82Y.

In some embodiments, the substitution at position 110 is selected from a group consisting of: E110A, E110K, E110G, E110V, E110F, E110M, E110W, E110P, E110Q, E110I, E 10S, E110T, E110N, E110D, E110R, E110H, E110L and E110Y.

In some embodiments, the substitution at position 111 is selected from a group consisting of: T111A, T111E, T111G, T111V, T111I, T111M, T111W, T111P, T111Q, T111K, T111S, T111F, T111N, T111D, T111R, T111H, T111L and T111Y.

In some embodiments, the substitution at position 122 is selected from a group consisting of: I122A, I122E, I122G, I122V, I122F, I122M, I122W, I122P, I122Q, I122K, I122S, I122T, I122N, I122D, I122R, I122H, I122L and I122Y.

In some embodiments, the substitution at position 125 is selected from a group consisting of: C125A, C125E, C125G, C125V, C125F, C125M, C125W, C125P, C125Q, C125K, C125I, C125S, C125T, C125N, C125D, C125R, C125H, C125L and C125Y.

In some embodiments, the substitution at position 127 is selected from a group consisting of: S127A, S127E, S127G, S127V, S127F, S127M, S127W, S127P, S127Q, S127K, S127I, S127T, S127N, S127D, S127R, S127H, S127L and S127Y.

In some embodiments, the substitution at position 128 is selected from a group consisting of: I128A, I128E, I128G, I128V, I128F, I128M, I128W, I128P, I128Q, I128K, I128S, I128T, I128N, I128D, I128R, I128H, I128L and I128Y.

In some embodiments, and the substitution at position 129 is selected from a group consisting of: I129A, I129E, I129G, I129V, I129T, I129M, I129W, I129P, I129Q, I129K, I129S, I129F, I129N, I129D, I129R, I129H, I129L and I129Y.

In some embodiments, the IL-2 variant comprises an amino acid sequence that differs from SEQ ID NO: 1 only by a C-terminal truncation. Specifically, the amino acid sequence of the IL-2 variant is selected from those as set forth in SEQ ID NOs: 2-10.

In some embodiments, the IL-2 variant comprises an amino acid sequence that differs from SEQ ID NO: 1 only by a substitution at position 38. Specifically, the amino acid sequence of the IL-2 variant is as set forth in SEQ ID NO: 22.

In some embodiments, the IL-2 variant comprises an amino acid sequence that differs from SEQ ID NO: 1 only by a substitution at position 42. Specifically, the amino acid sequence of the IL-2 variant is selected from those as set forth in SEQ ID NOs: 11-13.

In some embodiments, the IL-2 variant comprises an amino acid sequence that differs from SEQ ID NO: 1 only by a substitution at position 111.

In some embodiments, the IL-2 variant comprises an amino acid sequence that differs from SEQ ID NO: 1 only by a substitution at position 129.

In some embodiments, the amino acid sequence of the IL-2 variant comprises a C-terminal truncation in combination with a substitution at position 38. Specifically, the amino acid sequence of the IL-2 variant is selected from those as set forth in SEQ ID NO: 19 and homologous sequences thereof with at least 95% identity (e.g. at least 96% identity, 97% identity, 98% identity, or 99% identity).

In some embodiments, the amino acid sequence of the IL-2 variant comprises a C-terminal truncation in combination with a substitution at position 42. Specifically, the amino acid sequence of the IL-2 variant is selected from those as set forth in SEQ ID NOs: 14-18 and homologous sequences thereof with at least 95% identity (e.g. at least 96% identity, 97% identity, 98% identity, or 99% identity).

In some embodiments, the amino acid sequence of the IL-2 variant comprises a C-terminal truncation in combination with a substitution at position 111. Specifically, the amino acid sequence of the IL-2 variant is selected from those as set forth in SEQ ID NOs: 20-21 and homologous sequences thereof with at least 95% identity (e.g. at least 96% identity, 97% identity, 98% identity, or 99% identity).

In some embodiments, the amino acid sequence of the IL-2 variant comprises a C-terminal truncation in combination with a substitution at position 129. Specifically, the amino acid sequence of the IL-2 variant is selected from those as set forth in SEQ ID NOs: 23-27 and homologous sequences thereof with at least 95% identity (e.g. at least 96% identity, 97% identity, 98% identity, or 99% identity).

In some embodiments, the amino acid sequence of the IL-2 variant comprises a C-terminal truncation in combination with a substitution at positions 129 and 42. Specifically, the amino acid sequence of the IL-2 variant is selected from those as set forth in SEQ ID NOs: 28-29 and homologous sequences thereof with at least 95% identity (e.g. at least 96% identity, 97% identity, 98% identity, or 99% identity).

In some embodiments, the amino acid sequence of the IL-2 variant comprises a C-terminal truncation in combination with a substitution at positions 129 and 38. Specifically, the amino acid sequence of the IL-2 variant is selected from those as set forth in SEQ ID NOs: 30 and homologous sequences thereof with at least 95% identity (e.g. at least 96% identity, 97% identity, 98% identity, or 99% identity).

In some embodiments, the amino acid sequence of the IL-2 variant comprises a C-terminal truncation in combination with a substitution at position 127. Specifically, the amino acid sequence of the IL-2 variant is selected from those as set forth in SEQ ID NOs: 31-33 and homologous sequences thereof with at least 95% identity (e.g. at least 96% identity, 97% identity, 98% identity, or 99% identity).

In some embodiments, the amino acid sequence of the IL-2 variant comprises a C-terminal truncation in combination with a substitution at positions 127 and 129. Specifically, the amino acid sequence of the IL-2 variant is selected from those as set forth in SEQ ID NOs: 34 and homologous sequences thereof with at least 95% identity (e.g. at least 96% identity, 97% identity, 98% identity, or 99% identity).

In some embodiments, the amino acid sequence of the IL-2 variant comprises a C-terminal truncation in combination with a substitution at position 125. Specifically, the amino acid sequence of the IL-2 variant is selected from those as set forth in SEQ ID NOs: 35 and homologous sequences thereof with at least 95% identity (e.g. at least 96% identity, 97% identity, 98% identity, or 99% identity).

In some embodiments, the amino acid sequence of the IL-2 variant comprises a C-terminal truncation in combination with a substitution at position 128. Specifically, the amino acid sequence of the IL-2 variant is selected from those as set forth in SEQ ID NOs: 36 and homologous sequences thereof with at least 95% identity (e.g. at least 96% identity, 97% identity, 98% identity, or 99% identity).

In some embodiments, the amino acid sequence of the IL-2 variant comprises a C-terminal truncation in combination with a substitution at position 110. Specifically, the amino acid sequence of the IL-2 variant is selected from those as set forth in SEQ ID NOs: 37-39 and homologous sequences thereof with at least 95% identity (e.g. at least 96% identity, 97% identity, 98% identity, or 99% identity).

In some embodiments, the amino acid sequence of the IL-2 variant comprises a C-terminal truncation in combination with a substitution at position 122. Specifically, the amino acid sequence of the IL-2 variant is selected from those as set forth in SEQ ID NOs: 40-42 and homologous sequences thereof with at least 95% identity (e.g. at least 96% identity, 97% identity, 98% identity, or 99% identity).

In some embodiments, the amino acid sequence of the IL-2 variant comprises a C-terminal truncation in combination with a substitution at position 28. Specifically, the amino acid sequence of the IL-2 variant is selected from those as set forth in SEQ ID NOs: 43 and homologous sequences thereof with at least 95% identity (e.g. at least 96% identity, 97% identity, 98% identity, or 99% identity).

In some embodiments, the amino acid sequence of the IL-2 variant comprises a C-terminal truncation in combination with a substitution at position 32. Specifically, the amino acid sequence of the IL-2 variant is selected from those as set forth in SEQ ID NOs: 44 and homologous sequences thereof with at least 95% identity (e.g. at least 96% identity, 97% identity, 98% identity, or 99% identity).

In some embodiments, the amino acid sequence of the IL-2 variant comprises a C-terminal truncation in combination with a substitution at position 52. Specifically, the amino acid sequence of the IL-2 variant is selected from those as set forth in SEQ ID NOs: 45 and homologous sequences thereof with at least 95% identity (e.g. at least 96% identity, 97% identity, 98% identity, or 99% identity).

In some embodiments, the amino acid sequence of the IL-2 variant comprises a C-terminal truncation in combination with a substitution at position 76. Specifically, the amino acid sequence of the IL-2 variant is selected from those as set forth in SEQ ID NOs: 46 and homologous sequences thereof with at least 95% identity (e.g. at least 96% identity, 97% identity, 98% identity, or 99% identity).

In some embodiments, the amino acid sequence of the IL-2 variant comprises a C-terminal truncation in combination with a substitution at position 78. Specifically, the amino acid sequence of the IL-2 variant is selected from those as set forth in SEQ ID NOs: 47 and homologous sequences thereof with at least 95% identity (e.g. at least 96% identity, 97% identity, 98% identity, or 99% identity).

In some embodiments, the amino acid sequence of the IL-2 variant comprises a C-terminal truncation in combination with a substitution at position 82. Specifically, the amino acid sequence of the IL-2 variant is selected from those as set forth in SEQ ID NOs: 48 and homologous sequences thereof with at least 95% identity (e.g. at least 96% identity, 97% identity, 98% identity, or 99% identity).

In some embodiments, the amino acid sequence of the IL-2 variant comprises a C-terminal truncation in combination with a substitution at positions 110 and 129. Specifically, the amino acid sequence of the IL-2 variant is selected from those as set forth in SEQ ID NOs: 49-51 and homologous sequences thereof with at least 95% identity (e.g. at least 96% identity, 97% identity, 98% identity, or 99% identity).

In some embodiments, the amino acid sequence of the IL-2 variant comprises a C-terminal truncation in combination with a substitution at positions 122 and 129. Specifically, the amino acid sequence of the IL-2 variant is selected from those as set forth in SEQ ID NOs: 52-54 and homologous sequences thereof with at least 95% identity (e.g. at least 96% identity, 97% identity, 98% identity, or 99% identity).

In some embodiments, the amino acid sequence of the IL-2 variant comprises a C-terminal truncation in combination with a substitution at positions 28 and 129. Specifically, the amino acid sequence of the IL-2 variant is selected from those as set forth in SEQ ID NOs: 55 and homologous sequences thereof with at least 95% identity (e.g. at least 96% identity, 97% identity, 98% identity, or 99% identity).

In some embodiments, the amino acid sequence of the IL-2 variant comprises a C-terminal truncation in combination with a substitution at positions 32 and 129. Specifically, the amino acid sequence of the IL-2 variant is selected from those as set forth in SEQ ID NOs: 56 and homologous sequences thereof with at least 95% identity (e.g. at least 96% identity, 97% identity, 98% identity, or 99% identity).

In some embodiments, the amino acid sequence of the IL-2 variant comprises a C-terminal truncation in combination with a substitution at positions 52 and 129. Specifically, the amino acid sequence of the IL-2 variant is selected from those as set forth in SEQ ID NOs: 57 and homologous sequences thereof with at least 95% identity (e.g. at least 96% identity, 97% identity, 98% identity, or 99% identity).

In some embodiments, the amino acid sequence of the IL-2 variant comprises a C-terminal truncation in combination with a substitution at positions 76 and 129. Specifically, the amino acid sequence of the IL-2 variant is selected from those as set forth in SEQ ID NOs: 58 and homologous sequences thereof with at least 95% identity (e.g. at least 96% identity, 97% identity, 98% identity, or 99% identity).

In some embodiments, the amino acid sequence of the IL-2 variant comprises a C-terminal truncation in combination with a substitution at positions 78 and 129. Specifically, the amino acid sequence of the IL-2 variant is selected from those as set forth in SEQ ID NOs: 59 and homologous sequences thereof with at least 95% identity (e.g. at least 96% identity, 97% identity, 98% identity, or 99% identity).

In some embodiments, the amino acid sequence of the IL-2 variant comprises a C-terminal truncation in combination with a substitution at positions 82 and 129. Specifically, the amino acid sequence of the IL-2 variant is selected from those as set forth in SEQ ID NOs: 60 and homologous sequences thereof with at least 95% identity (e.g. at least 96% identity, 97% identity, 98% identity, or 99% identity).

The binding affinity of the IL-2 variant to at least one of IL-2Rα, IL-2Rβ and common γ chain may be measured by FACS binding analysis.

In some aspects, the present disclosure provides a fusion protein, comprising the IL-2 variant as described above operably linked to a non-IL-2 moiety. Preferably, the non-IL-2 moiety is capable of extending the half-life of IL-2 variant in vivo. The fusion protein may be a monomer, dimer or multimer. In some embodiments, the non-IL-2 moiety is selected from an Fc region, a human serum albumin, an anti-human serum albumin moiety, PEGs, and lipids.

In some embodiments, the fusion protein comprises one or more (e.g. 2, 3, 4, 6, 8 or more) IL-2 variants as disclosed herein, wherein the IL-2 variant may have a same or different amino acid sequence.

In some embodiments, the fusion protein comprises two amino acid chains, wherein:
(a) each chain comprises one IL-2 variant fused to one chain of the Fc region at the N-terminal of the Fc region;
(b) each chain comprises one IL-2 variant fused to one chain of the Fc region at the C-terminal of the Fc region;
(c) one chain comprises one IL-2 variant fused to one chain of the Fc region at the N-terminal of the Fc region, and the other chain comprises one IL-2 variant fused to the other chain of the Fc region at the C-terminal of the Fc region; or
(d) each chain comprises two IL-2 variants fused to the N-terminal and C-terminal of one chain of the Fc region, respectively.

In some embodiments, each chain of the fusion proteins as described above comprises more than one IL-2 variants that are operably linked together, and then fused to the N or C terminal of the Fc region. Preferably, the two chains have the same number of IL-2 variants to form a symmetric configuration.

In some exemplary embodiments, the fusion protein as disclosed herein comprises the amino acid sequence as set forth in SEQ ID NO: 138 or 139.

In some aspects, the present disclosure provides a bifunctional polypeptide complex, comprising an IL-2 moiety, an antigen-binding moiety, a hinge region and an Fc region, wherein: the IL-2 moiety comprises a wild-type IL-2 or the IL-2 variant as described above, the antigen-binding moiety is selected from a Fab, a VHH, a scFv, an aptamer, and a TCR, and wherein the antigen-binding moiety is operably linked to the N-terminal of the hinge region and Fc region or the C-terminal of the Fc region, and the IL-2 moiety is operably linked to the N-terminal of the hinge region or the C-terminal of the Fc region. The polypeptide complex may be homodimeric or heterodimeric.

In some embodiments, the bifunctional polypeptide complex comprises two heavy chains and one light chain, wherein the antigen-binding moiety is a Fab, and the first heavy chain comprises, from N-terminal to C-terminal:
(a) the IL-2 moiety;
(b) optionally, a linker; and
(c) one chain of the hinge region and the Fc region;
the second heavy chain comprises, from N-terminal to C-terminal:
(d) heavy chain of the Fab; and
(e) the other chain of the hinge region and the Fc region, and
the light chain comprises light chain of the Fab.

In some embodiments, the bifunctional polypeptide complex comprises two chains, wherein the antigen-binding moiety is a VHH, and the first chain comprises, from N-terminal to C-terminal:
(a) the IL-2 moiety;
(b) optionally, a linker; and
(c) one chain of the hinge region and the Fc region;
the second chain comprises, from N-terminal to C-terminal:
(d) the VHH; and
(e) the other chain of the hinge region and the Fc region.

In some embodiments, the polypeptide complex comprises two heavy chains and two light chains, wherein the antigen-binding moiety is a Fab, and the first heavy chain comprises, from N-terminal to C-terminal:
(a) heavy chain of the Fab;
(b) one chain of the hinge region and the Fc region;
(c) optionally, a linker; and
(d) the IL-2 moiety, and the second heavy chain comprises, from N-terminal to C-terminal:
(e) heavy chain of the Fab; and
(f) the other chain of the hinge region and the Fc region; and the two light chains each comprising light chain of the Fab.

In some embodiments, the bifunctional polypeptide complex comprises two chains, wherein the antigen-binding moiety is a VHH, and the first chain comprises, from N-terminal to C-terminal:
(a) the VHH;
(b) one chain of the hinge region and the Fc region;
(c) optionally, a linker; and
(d) the IL-2 moiety, and
the second chain comprises, from N-terminal to C-terminal:
(e) the VHH; and
(f) the other chain of the hinge region and the Fc region.

In some embodiments, the bifunctional polypeptide complex comprises two heavy chains and two light chains, wherein the antigen-binding moiety is a Fab, and
the two heavy chains each comprising, from N-terminal to C-terminal:
(a) heavy chain of the Fab;
(b) one chain of the hinge region and the Fc region;
(c) optionally, a linker; and
(d) the IL-2 moiety, and
the two light chains each comprising light chain of the Fab.

In some embodiments, the bifunctional polypeptide complex comprises two chains, wherein the antigen-binding moiety is a VHH, and
each chain comprises, from N-terminal to C-terminal:
(a) the VHH;
(b) one chain of the hinge region and the Fc region;
(c) optionally, a linker; and
(d) the IL-2 moiety.

In some embodiments, the bifunctional polypeptide complex comprises two heavy chains and two light chains, wherein the antigen-binding moiety is a Fab, and
the two heavy chains each comprising, from N-terminal to C-terminal:
(a) the IL-2 moiety
(b) optionally, a linker; and
(c) one chain of the hinge region and the Fc region;
(d) heavy chain of the Fab, and
the two light chains each comprising light chain of the Fab.

In some embodiments, the bifunctional polypeptide complex comprises two chains, wherein the antigen-binding moiety is a VHH, and
each chain comprises, from N-terminal to C-terminal:
(a) the IL-2 moiety;
(b) optionally, a linker; and
(c) one chain of the hinge region and the Fc region;
(d) the VHH.

In some embodiments, the bifunctional polypeptide complex comprises two chains, wherein the antigen-binding moiety is a scFv, and
each chain comprises, from N-terminal to C-terminal:
(a) the IL-2 moiety;
(b) optionally, a linker; and
(c) one chain of the hinge region and the Fc region;
(d) the scFv.

In some embodiments, the antigen-binding moiety specifically binds to a target antigen, which may be selected from PD-1, PD-L1, PD-L2, CTLA-4, LAG3, TIM-3, TIM4, 4-1BB, OX-40, OX-40L, GITR, A2aR, TIGIT, CD96, PVRIG, CD226, 5T4, VISTA, VSIG3, VSIG4, ICOS, CD28, CD3, CD4, CD8, CD45, CD44v6, CD27, CD47, SIRPAa, SLAMF7, CD24, Siglec10, Sigleci5, Siglec8, VSIR, VSIG4, PSGL-1, C5AR1, BTN1A1, BTN3A1, CD70, RANKL, CSF1R, CSF2RB, TNFRSF1/1a/1b, BDCA2, BTLA, C5aR, NKG2A, NKG2D, NKp30, NKp46, CD16a, CD56, CD166, FCGR3, CD2, Neurophilin-1, CCR8, CCR2, CCR4, CCR5, CCR6, CCR7, CCR8, GCGR, CXCR2, CXCR4, CXCR5, CALCRL, ETAR, GLP1R, CX3CR1, GPR1, GPR17, GPR20, GPR30, GPR34, GPR-65, GPCR78, GPRC5D, GPR84, LGR4, LGR5, VEGF, VEGFR, HER2, HER3, Trop2, pCAD, ERα, EGFR, de2-7 EGFR, EGFRvIII, PSMA, PSCA, PSA, TAG-72, SEZ6, SEZ6L, SEZ6L2, SEMA4D, DLL3, GD2, GPC3, KLB, KLRB1, KLRG1, GPC1, PCSK9, EpCAM, p-Cadherin, Caludin 6, Caludin 18.2, FGFR2b, FGFR3, FGFR4, MUC1, MUC13, MUC16, MUC17, MUCL3, FolRa, TfR, TF, TFR, TFPI, c-Met, NY-ESO-1, GUCY2C, LIV-1, Integrin αvβ6, Integrin α10β1, Intergrin α3, Integrin α5β4, Integrin αvβ3, Integrin αvβ8, ROR1, ROR2, PRLR, PTK7, B7-H3, Nectin-4, NetG1, Ax1, CD147, LRRC15, Napi2b, STEAP1, LY6G6D, LYPD1, MACRO, MerTK, MICA, MICB, MSLN, Mkars, G12D, CDH3, CDH6, CDH17, APLA2, CAIX, CD46, CD47, CLDN6, EphA3, Fucosyl-GM1, ITGA3, Kallikrein, MISRII, Podocalyxin, RON, ROBO1, PAUF, PLA2, Podocalyxin, PRLR, PTK7, TM4SF1, TMEFF2, TREAKR, TREM-1, TREM-2, uPARAP, TYRP1, KAAG1, RU2AS, CD146, CD63, Endoglin, Globo H, IGF-1R, TEM1, TEM8, TAXIBP3, ADAM-9, ENPP3, EphA2, EphA3, FcRH5, NaPi3b, TWEAK, DLK1, SORT1, SSTR2, STEAP1, CD25, CD39, GARP, LRRC33, LAIR1, LAMP3, LAP, LEPR, LILRB1, LILRB2, LILRB4, RAGE, FGL1, TPBG, PDGFRB, TGFBR2, CEACAM1, CEACAM5, CEACAM6, Carcinoembryonic antigen (CEA), ICAM1, A33, CAMPATH-1 (CDw52), Carboanhydrase IX (MN/CA IX), CD248, PDPN, ITGB1, ITGAV, CD20, CD19, CD21, CD22, CLL, BCMA, DCLK1, DDR1, DLK1, DPEP3, DKK1, CD5, CD13, CD30, CD33, CD34, CD36, CD37, CD38, CD43, CD52, CD55, CD94, CD99, CD7, CD71, CD73, CD74, CD79a, CD79b, CD229, CD132, CD133, G250, CSF1R (CD115), HLA-DR, HLA-G, HTRA1, TRA-1-60, IGFR, IL-2 receptor, MCSP (Melanoma-associated cell surface chondroitin sulphate proteoglycane), ART1, ASGR1, B7H3, B7-H4, B7H6, CD124, c-Kit (CD117), CD7, Clex12A, Clever-1, IL-13RA2, IL-11RA, IL-31RA, IL-4RA, IFNAR, ActRIIb, IL-7R, SLAMF7, Fms-like tyrosine kinase 3 (FLT-3, CD135), GFRA1, BTLA, GloboH, CSF2RB, chondroitin sulfate proteoglycan 4 (CSPG4, melanoma-associated chondroitin sulfate proteoglycan), ITGA4, Clec5a, Clec7a, Clec9a, Clec12a, CLEC14, CD205, CD206, CD200R1, CD228, CD229, CD40, CD40L, FcRn, TLR8, TLR9, TNFR2, LTBR, CD44, CD93, PDGF, PDGFR-alpha (CD140a), PDGFR-beta (CD140b), CD146, CD147, CRTH2, TNF-α, TGF-3, IL1RAcP, TSLP, DR5, ST2, fibroblast activating protein (FAP), CDCP1, Derlin1, Tenascin, frizzled 1-10, the vascular antigens VEGFR2 (KDR/FLK1), VEGFR3 (FLT4, CD309), Endoglin, Tie2 and other tumor associated antigens (TAAs), I/O checkpoints, tumor microenvironment targets, or autoimmune and inflammatory diseases associated targets.

In some embodiments, the linker is (G4S)n linker with n=0-5.

In some embodiments, the hinge region is a truncated hinge region compared to wild-type hinge region. For example, the hinge region has a truncation of 1 to 10 amino acids from the N-terminal or C-terminal, such as a truncation of 10, 9, 8, 7, 6, 5, 4, 3, 2 or 1 amino acid(s).

In some embodiments, the Fc region is an IgG1, IgG2 or IgG4 Fc region. The IgG1 Fc region may have a LALA mutation, and the IgG4 Fc region may have a FALA mutation. Alternatively/additionally, each chain of the Fc variant comprises one or more substitutions compared to wild type human Fc for various purposes, for example to eliminate effector function (such as D265A), to promote heterodimerization (such as a knob into hole structure, charge interaction, single chain Fc), or to change half-life (such as YTE, LS or Ala-scan mutations), or to remove N-glycosylation (such as N297D).

In some embodiments, the polypeptide complex comprises:
(a) a first heavy chain that comprises an amino acid sequence selected from those set forth in SEQ ID NOs: 62-130 and homologous sequences thereof with at least 95% identity;
(b) a second heavy chain that comprises an amino acid sequence selected from those set forth in SEQ ID NOs: 131-133 and homologous sequences thereof with at least 95% identity; and
(c) a light chain that comprises an amino acid sequence selected from those set forth in SEQ ID NO: 134 and homologous sequences thereof with at least 95% identity.

In some specific embodiments, the polypeptide complex as disclosed herein comprises:
a first heavy chain that comprises an amino acid sequence selected from those set forth in SEQ ID NOs: 62, 63, 65 and 67, a second heavy chain that comprises an amino acid sequence selected from those set forth in SEQ ID NOs: 131, and a light chain that comprises an amino acid sequence selected from those set forth in SEQ ID NO: 134.

In some specific embodiments, the polypeptide complex as disclosed herein comprises:
a first heavy chain that comprises an amino acid sequence selected from those set forth in SEQ ID NOs: 64, 66 and 68-95, a second heavy chain that comprises an amino acid sequence selected from those set forth in SEQ ID NO: 132, and a light chain that comprises an amino acid sequence selected from those set forth in SEQ ID NO: 134.

In some specific embodiments, the polypeptide complex as disclosed herein comprises:
a first heavy chain that comprises an amino acid sequence selected from those set forth in SEQ ID NOs: 96-130, a second heavy chain that comprises an amino acid sequence selected from those set forth in SEQ ID NO: 133, and a light chain that comprises an amino acid sequence selected from those set forth in SEQ ID NO: 134.

In some aspects, the present disclosure provides an isolated nucleic acid molecule, comprising a nucleic acid sequence encoding the heavy chain and/or light chain of the polypeptide complex as disclosed herein.

In some aspects, the present disclosure provides a vector comprising such nucleic acid, and a host cell comprising the vector as disclosed herein.

In some aspects, the present disclosure provides a pharmaceutical composition comprising the polypeptide complex as disclosed herein and a pharmaceutically acceptable carrier.

In some aspects, the present disclosure provides an immunoconjugate comprising the IL-2 variant as disclosed herein or the fusion protein as disclosed herein conjugated to an agent, such as a chemotherapeutic agent, radioactive particle or toxin.

In some aspects, the present disclosure provides a method for producing the polypeptide complex as disclosed herein comprising the steps of:
expressing the polypeptide complex in the host cell comprising a vector(s) encoding the heavy chain and/or light chain of the polypeptide complex; and
isolating the polypeptide complex from the host cell culture.

In some aspects, the present disclosure provides a method of modulating an immune response in a subject, comprising administering the polypeptide complex as disclosed herein or the pharmaceutical composition as disclosed herein to the subject, optionally the immune response is NK cell, CD8+ cell, or CD4+ T cell (especially Treg) related.

In some aspects, the present disclosure provides a method for treating or preventing cancer in a subject, comprising administering an effective amount of the polypeptide complex as disclosed herein or the pharmaceutical composition as disclosed herein to the subject.

In some aspects, the present disclosure provides a method for treating or preventing cancer in a subject, comprising administering an effective amount of a DNA or RNA encoding the IL-2 variant or the polypeptide complex as disclosed herein to the subject.

In some embodiments, the method further comprises administering an additional anti-tumor therapy, such as cell immunotherapy including tumor-infiltrating lymphocyte (TIL) therapy, T cell receptor (TCR) therapy, chimeric antigen receptor (CAR) T cell therapy, macrophage cell therapy, and NK cell therapy, as well as targeted therapy and chemotherapy.

In some embodiments, the method further comprises administering an additional anti-tumor therapy, such as gene therapy including lentivirus, AAV, poxvirus, herpes zoster virus, oncolytic virus and other RNA/DNA vector encoding IL-2 variants.

In some embodiments, the cancer is selected but not limited from breast cancer, lung cancer, colon cancer, ovarian cancer, melanoma, bladder cancer, renal cell carcinoma, liver cancer, prostate cancer, stomach cancer, pancreatic cancer, NSCLC, non-Hodgkin's lymphoma, chronic lymphocytic leukemia, diffuse large B-cell lymphoma, and multiple myeloma.

In some aspects, the present disclosure provides use of the polypeptide complex as disclosed herein in the manufacture of a medicament for treating or preventing cancer.

In some aspects, the present disclosure provides the polypeptide complex as disclosed herein for use as a medicament.

In some aspects, the present disclosure provides the polypeptide complex as disclosed herein for use in treating or preventing cancer.

In some aspects, the present disclosure provides a kit for treating or diagnosing cancer, comprising a container comprising the polypeptide complex as disclosed herein or the immunoconjugate as disclosed herein.

In some aspects, the present disclosure provides the polypeptide complex as disclosed herein for use in treating or preventing autoimmune or inflammatory diseases.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein.

### BRIEF DESCRIPTION OF THE FIGURES

FIG. 1 illustrates a schematic description of various fusion protein and polypeptide complex formats comprising wild type IL-2 or an IL-2 variant, according to some embodiments of the disclosure. The conic shape indicates the IL-2 moiety, the ladder shape indicates the hinge region, and the oval shape indicates the antigen-binding moiety and Fc region. The upper row shows polypeptide complexes comprising an IL-2 moiety and an antigen-binding moiety: one IL-2 moiety+one antigen moiety (a, Fab; b, VHH) both at N-terminal; one IL-2 moiety at C-terminal+two antigen moieties (c, Fab; d, VHH) at N-terminal; two IL-2 moieties at C-terminal+two antigen moieties (e, Fab; f, VHH) at N-terminal; two IL-2 moieties at N-terminal+two antigen moieties (t, Fab; u, VHH; v, scFv) at C-terminal. These formats can accommodate different antigen-binding moieties such as Fabs, VHHs and scFvs comprising variable regions targeted to different epitopes/antigens. The second and third row shows fusion proteins or polypeptide complexes comprising one, two, four or more IL-2 moieties fused to the Fc region: (m) one IL-2 moiety fused to a non-IL-2 moiety (circle shape), single chain; (g, n) two IL-2 moieties at the N-terminal; (h, o) two IL-2 moieties at the C-terminal; (i, p) one IL-2 moiety at the N-terminal and one IL-2 moiety at the C-terminal; (j, q) two IL-2 moieties at the N-terminal and two IL-2 moieties at the C-terminal; (k, r) four or more IL-2 moieties at the N-terminal; (1, s) four or more IL-2 moieties at the C-terminal. n1 and n2 represent repeats of the IL-2 moiety. The “knob” in the Fc region indicates the two chains of the Fc region are modified to promote dimerization. When comprising more than one IL-2 moieties, the IL-2 moieties may be the same or different.

FIG. 2 illustrates the binding curves in NK92 (a) and HH (b) cells following treatment with wt IL-2 fused on N or C terminal of Fc.

FIG. 3 illustrates the binding curves in NK92 (a) and HH (b) cells treated with polypeptide complexes comprising wt IL-2 in human IgG1 or IgG4 format.

FIG. 4 illustrates the median fluorescence intensity (MFI) of STAT5 phosphorylation on primary human CD8+ T cells followed by treatment with polypeptide complexes comprising wt IL-2 or IL-2 variants in different formats.

FIG. 5 illustrates polypeptide complexes comprising truncated IL-2 variants showed different affinity and potency in cells with IL-2Rβ/γ. (a) The MFI of STAT5 phosphorylation on primary human CD8+ T cells followed by treatment with IL-2 variants. (b) The binding curves of IL-2 variants in HH cells.

FIG. 6 illustrates polypeptide complexes comprising truncated IL-2 variants showed different affinity and potency in cells with IL-2Rα/β/γ. (a) The MFI of STAT5 phosphorylation on human Treg cells after treatment with IL-2 variants. (b) The binding curve of IL-2 variants in NK92 cells.

FIG. 7 illustrates truncated IL-2 variants with amino acid mutation showed different affinity and potency in cells expressing IL-2Rβ/γ. (a) The median fluorescence intensity (MFI) of STAT5 phosphorylation on primary human CD8+ T cells followed by treatment with IL-2 variants. (b) The binding curves in HH cells following treatment with IL-2 variants.

FIG. 8 illustrates truncated IL-2 variants with amino acid mutation showed similar affinity and potency in cells expressing IL-2Rα/β/γ. (a) The median fluorescence intensity (MFI) of STAT5 phosphorylation on human Treg cells followed by treatment with IL-2 variants. (b) The binding curves in MJ20 cells following treatment with IL-2 variants.

FIG. 9 illustrates IL-2 variants showed different affinity and potency in cells expressing IL-2Rβ/γ. (a) The median fluorescence intensity (MFI) of STAT5 phosphorylation on primary human CD8+ T cells followed by treatment with IL-2 variants. (b) The binding curves in HH cells following treatment with IL-2 variants.

FIG. 10 illustrates IL-2 variants showed similar affinity and potency in cells expressing IL-2Rα/β/γ. (a) The median fluorescence intensity (MFI) of STAT5 phosphorylation on human Treg cells followed by treatment with IL-2 variants. (b) The binding curves in MJ20 cells following treatment with IL-2 variants.

FIG. 11 illustrates IL-2 variants with amino acid mutation in IL-2Rα interface showed similar affinity and potency in cells expressing IL-2Rα/β/γ. (a) The median fluorescence intensity (MFI) of STAT5 phosphorylation on human Treg cells followed by treatment with IL-2 variants. (b) The binding curves in NK92 cells following treatment with IL-2 variants.

FIG. 12 illustrates IL-2 variants with amino acid mutation in IL-2Rα interface showed different affinity and potency in cells expressing IL-2Rβ/γ. (a) The median fluorescence intensity (MFI) of STAT5 phosphorylation on primary human CD8+ T cells followed by treatment with IL-2 variants. (b) The binding curves in HH cells following treatment with IL-2 variants.

FIG. 13 illustrates the binding curves in HH (a) and NK92 (b) cells post treatment with IL-2 variants.

FIG. 14 illustrates IL-2 variants showed different potency in primary human CD8+ T cells (a) and Treg (b), determined by STAT5 phosphorylation assay.

FIG. 15 illustrates the binding curves in HH (a) and MJ20 (b) cells post treatment with IL-2 variants.

FIG. 16 illustrates IL-2 variants showed different potency in primary human CD8+ T cells (a) and Treg (b), determined by STAT5 phosphorylation assay.

FIG. 17 illustrates the binding curves in HH (a) and MJ20 (b) cells post treatment with bivalent IL-2 variants.

FIG. 18 illustrates bivalent IL-2 variants showed different potency in primary human CD8+ T cells (a) and Treg (b), determined by STAT5 phosphorylation assay.

FIG. 19 illustrates SPR curves showing binding of pre-complexed human IL-2Rβ with IL-2 variants (1:1) to human IL-2Rγc immobilized on a sensor chip.

FIG. 20 illustrates body weight change of maximum tolerance dose (MTD) assay. Mice were treated with IL-2 variants in different doses: W3XX115-T2U0.E44-1.uIgG4V322 and W3XX115-T3U0.E44-1.uIgG4V322 (a); W3XX115-T2U0.E44-6.uIgG4V322, W3XX115-T2U0.E44-15.uIgG4V322 and W3XX115-T3U0.E44-20.uIgG4V322 (b); W3XX115-T2U0.E44-26.uIgG4V322 and W3XX115-T3U0.E44-33.uIgG4V322 (c); W3XX115-T2.Z20-1.uIgG4V322 and W3XX115-T2.Z20-2.uIgG4V322 (d).

FIG. 21 illustrates the change in tumor volume of MC38 colon carcinoma model after treated with IL-2 variants. MC38 syngeneic model was treated by W3XX115-T2U0.E44-1.uIgG4V322 and W3XX115-T3U0.E44-1.uIgG4V322 (a); treated by IL-2 variants in MTD or high dose (b), or in different dose range (c, d).

FIG. 22 illustrates pharmacodynamics effect of Treg and CD8+ T cells in tumor upon treatment of W3XX115-T2U0.E44-1.uIgG4V322 and W3XX115-T3U0.E44-1.uIgG4V322.

FIG. 23 (a)-(1) illustrate pharmacodynamics effect of different kinds of tumor infiltrated immune cells upon treatment of IL-2 variants.

FIG. 24 (a)-(f) illustrate pharmacodynamics effect of peripheral T cells upon treatment of IL-2 variants.

FIG. 25 illustrates the change of body weight (a) and colon length (b) of DSS-induced colitis mice model upon treatment of IL-2 variants.

### DETAILED DESCRIPTION

While the present disclosure may be embodied in many different forms, disclosed herein are specific illustrative embodiments thereof that exemplify the principles of the disclosure. It should be emphasized that the present disclosure is not limited to the specific embodiments illustrated. Moreover, any section headings used herein are for organizational purposes only and are not to be construed as limiting the subject matter described.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like. In this application, the use of “or” means “and/or” unless stated otherwise. Furthermore, the use of the term “comprising,” as well as other forms, such as “comprises” and “comprised,” is not limiting. In addition, ranges provided in the specification and appended claims include both end points and all points between the end points.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated. See, e.g., Abbas et al., Cellular and Molecular Immunology, 6^{th} ed., W.B. Saunders Company (2010); Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual, Cold Spring Harbor Laboratory Press, Cold Spring Harbor, N.Y. (1998); and Coligan et al., Short Protocols in Protein Science, Wiley, John & Sons, Inc. (2003). The nomenclature used in connection with, and the laboratory procedures and techniques of, analytical chemistry, synthetic organic chemistry, and medicinal and pharmaceutical chemistry described herein are those well-known and commonly used in the art.

### Definitions

In order to better understand the disclosure, the definitions and explanations of the relevant terms are provided as follows.

The term “IL-2” or “Interleukin-2”, as used herein, is intended to encompass any form of IL-2, for example, 1) native unprocessed IL-2 molecule, “full-length” IL-2 protein or naturally occurring variants of IL-2; 2) any form of IL-2 that results from processing in the cell; or 3) full length, a fragment (e.g., a truncated form) or a modified form. IL-2 is a cytokine mainly produced from activated T cells and contributes to proliferation and activation of various immune cells. Human mature IL-2 has a molecular weight of about 15 kDa (133 amino acids, as shown in SEQ ID NO: 1) and has a four-α-helix bundle structure. As used in the disclosure, “IL-2” may be either wild-type IL-2 or an IL-2 variant.

The term “variant”, with regard to polypeptide or protein, means a biologically active polypeptide which includes one or more amino acid mutations in the native protein sequence. Optionally, the one or more amino acid mutations include amino acid substitution, deletion, and/or insertion at certain positions in the amino acid sequence. A variant has at least about 80%, and preferably at least about 85%, more preferably at least about 90%, and even more preferably at least about 95% (e.g. at least 96%, 97%, 98%, or 99% or higher) amino acid sequence identity with the corresponding native sequence polypeptide. Such variants include, for instance, polypeptides wherein one or more amino acid (naturally occurring amino acid and/or a non-naturally occurring amino acid) residues are added, or deleted, at the N- and/or C-terminus of the polypeptide. Variants also include polypeptide fragments (e.g., subsequences, truncations, etc.), typically biologically active, of the native sequence.

The term “IL-2 variant”, as used herein, includes all proteins which are produced by adding any modifications to wild-type IL-2, and has a function similar to wild-type IL-2, such as a specific binding to IL-R2a, IL-2Rβ/γc, and/or the combined IL-R2α/β/γc complex (although preferably the binding affinity is attenuated compared to the wild-type IL-2), activation of immune cells such as T cells (e.g. CD8+ T cells, NK cells, and Treg cells), phosphorylation of STAT5, among others. Examples of the variants include an IL-2 variant in which the wt IL-2 is modified by an amino acid modification (for example, deletion, substitution or addition), an IL-2 variant in which the wt IL-2 is modified by saccharide modification, and IL-2 variant in which the wt IL-2 is modified by chemical modification.

The term “antibody” or “Ab” herein is used in the broadest sense, which encompasses various antibody structures, including polyclonal antibodies, monospecific and multispecific antibodies (e.g. bispecific antibodies) and the polypeptide complexes as disclosed herein. A native intact antibody generally is a Y-shaped tetrameric protein comprising two heavy (H) and two light (L) polypeptide chains held together by covalent disulfide bonds and non-covalent interactions. Light chains of an antibody may be classified into κ and λ light chain. Heavy chains may be classified into μ, δ, γ, α and ε, which define isotypes of an antibody as IgM, IgD, IgG, IgA and IgE, respectively. In a light chain and a heavy chain, a variable region is linked to a constant region via a “J” region of about 12 or more amino acids, and a heavy chain further comprises a “D” region of about 3 or more amino acids. Each heavy chain consists of a heavy chain variable region (V_{H}) and a heavy chain constant region (C_{H}). A heavy chain constant region consists of 3 domains (C_{H}1, C_{H}2 and C_{H}3). Each light chain consists of a light chain variable region (V_{L}) and a light chain constant region (C_{L}). V_{H} and V_{L} region can further be divided into hypervariable regions (called complementary determining regions (CDR)), which are interspaced by relatively conservative regions (called framework region (FR)). Each V_{H} and V_{L} consists of 3 CDRs and 4 FRs in the following order: FR1, CDR1, FR2, CDR2, FR3, CDR3, FR4 from N-terminal to C-terminal. The variable region (V_{H} and V_{L}) of each heavy/light chain pair forms antigen binding sites, respectively. Distribution of amino acids in various regions or domains follows the definition in Kabat Sequences of Proteins of Immunological Interest (National Institutes of Health, Bethesda, Md. (1987 and 1991)), Chothia & Lesk (1987) J. Mol. Biol. 196:901-917; Chothia et al., (1989) Nature 342:878-883, Contact numbering, AbM numbering or IMGT numbering. Antibodies may be of different antibody isotypes, for example, IgG (e.g., IgG1, IgG2, IgG3 or IgG4 subtype), IgA1, IgA2, IgD, IgE or IgM antibody. In a broad sense, the polypeptide complexes as disclosed herein comprising an antigen-binding moiety also belongs to an antibody.

The term “antigen-binding moiety” as used herein refers to an antibody fragment formed from a portion of an antibody comprising one or more CDRs, or any other antibody fragment that binds to an antigen but does not comprise an intact native antibody structure. Examples of antigen-binding moiety include, without limitation, a variable domain, a variable region, a diabody, a Fab, a Fab′, a F(ab′)2, an Fv fragment, a disulphide stabilized Fv fragment (dsFv), a (dsFv)2, a bispecific dsFv (dsFv-dsFv′), a disulphide stabilized diabody (ds diabody), a multispecific antibody, a camelized single domain antibody, a nanobody, a domain antibody, a VHH, a bivalent domain antibody and a TCR. The term “Fab”, as used herein, is meant the polypeptide that comprises the VH, CH1, VL and CL immunoglobulin domains. An antigen-binding moiety is capable of binding to the same antigen to which the parent antibody binds. In certain embodiments, an antigen-binding moiety may comprise one or more CDRs from a particular human antibody grafted to a framework region from one or more different human antibodies. For more and detailed formats of antigen-binding moiety are described in (Spiess et al., Molecular Immunology, 67(2), pp. 95-106 (2015), and Brinkman et al., mAbs, 9(2), pp. 182-212 (2017), which are incorporated herein by their entirety.

The term “hinge region”, as used herein, has a same meaning as used with regard to an antibody, which refers to a short sequence of the heavy chains (H) of immunoglobulins linking the Fab (Fragment antigen binding) region to the Fc (Fragment crystallizable) region. The hinge region may be a full or a partial hinge region. The hinge region may consist of at least 2 (e.g., 5, 10, 15, 20, 40, 60 or more) amino acids which result in a flexible or semi-flexible linkage between adjacent variable and/or constant domains in a single polypeptide molecule. In some embodiments, the hinge region comprised in the polypeptide complex as disclosed herein is a C-terminal or N-terminal truncated hinge region. It will be appreciated that the hinge region is a specific linker and may be suitably replaced by other linker sequences in constructing the fusion protein or polypeptide complex herein.

The term “Fc”, as used herein, has a same meaning as used with regard to an antibody, which refers to that portion of the antibody comprising the second (CH2) and third (CH3) constant regions of a first heavy chain bound to the second and third constant regions of a second heavy chain via disulfide bonding. The Fc region of the antibody is responsible for various effector functions such as ADCC and CDC, but generally does not function in antigen binding. In the present disclosure, the term “Fc” includes both wild-type Fc, Fc variants and grafted Fc.

The term “modification”, with respect to an amino acid residue/position as used herein, refers to a change of a primary amino acid sequence as compared to a starting amino acid sequence, wherein the change results from a sequence alteration involving said amino acid residue/positions. For example, typical modifications include substitution of the residue (or at said position) with another amino acid (e.g., a conservative or non-conservative substitution), insertion of one or more amino acids adjacent to said residue/position, and deletion of said residue/position. An “amino acid substitution”, or variation thereof, refers to the replacement of an existing amino acid residue in a predetermined (starting) amino acid sequence with a different amino acid residue. Generally, the modification results in alteration in at least one physicobiochemical activity of the variant polypeptide compared to a polypeptide comprising the starting (or “wild type”) amino acid sequence. For example, in an IL-2 variant, a physicobiochemical activity that is altered can be binding affinity, binding capability and/or binding effect upon a target molecule. As used herein, two or more substitutions in an amino acid sequence may be expressed with “+” or “/” between each substitution.

As used herein, the term “fusion protein” refers to a chimeric polypeptide comprising an IL-2 moiety and a non-IL-2 moiety (and optionally more portions) operably linked together, where each of the portions is a polypeptide having a different property. The property may be a biological property, such as activity in vitro or in vivo. The property may also be a simple chemical or physical property, such as binding to a target antigen, catalysis of a reaction, etc. The two portions may be linked directly by a single peptide bond or through a peptide linker containing one or more amino acid residues. Generally, the two portions and the linker will be in reading frame with each other. Preferably, the non-IL-2 moiety can extend the half-life of IL-2 in vivo. In some embodiments, the non-IL-2 moiety is an immunoglobulin constant region comprising the hinge region and Fc region, thus the generated fusion protein is referred to as IL-2/Fc fusion protein. The IL-2/Fc fusion protein comprises the IL-2 moiety operably linked to the Fc region (optionally linked via a hinge region), and generally is a dimer.

The term “polypeptide complex” can be used interchangeably with “fusion protein” herein. The term “bifunctional polypeptide complex” refers to a fusion protein comprising an IL-2 moiety, an antigen-binding moiety, and an Fc region. Such fusion proteins are structurally similar to a conventional antibody, in view that the IL-2 moiety replaces the Fab or VHH in a conventional antibody or adds to the C terminal of the antibody. The term “bifunctional” means the polypeptide complex combined an antigen-binding specificity and IL-2 modulation effect.

The terms “operably linked” refer to a juxtaposition, with or without a spacer or linker, of two or more biological sequences of interest in such a way that they are in a relationship permitting them to function in an intended manner. When used with respect to polypeptides, it is intended to mean that the polypeptide sequences are linked in such a way that permits the linked product to have the intended biological function. For example, an antigen-binding moiety may be operably linked to a Fc region so as to provide for a stable product with antigen-binding activity. By “operably linked to”, the antigen-binding moiety may be directly linked to the Fc region as long as the two parts can function normally, or more preferably, the antigen-binding moiety may be indirectly linked to the Fc region via a linker sequence, such as a hinge region. The term may also be used with respect to polynucleotides. For one instance, when a polynucleotide encoding a polypeptide is operably linked to a regulatory sequence (e.g., promoter, enhancer, silencer sequence, etc.), it is intended to mean that the polynucleotide sequences are linked in such a way that permits regulated expression of the polypeptide from the polynucleotide.

The term “gene therapy”, as used herein, refers to a therapy that aims to treat diseases by replacing, inactivating or introducing genes into cells—either inside the body (in vivo) or outside of the body (ex vivo). In some embodiments, a DNA or RNA sequence encoding the IL-2 variant(s) as disclosed herein is administered to a subject. Gene therapy includes using lentivirus, AAV, poxvirus, herpes zoster virus, oncolytic virus and other RNA/DNA vectors to deliver the DNA or RNAs encoding the IL-2 variants.

The term “cell therapy”, as used herein, refers to a therapy that aims to treat diseases by restoring or altering certain sets of cells or by using cells to carry a therapy through the body. With cell therapy, cells are cultivated or modified outside the body before being injected into the patient. The cells may originate from the patient (autologous cells) or a donor (allogeneic cells). Current cell therapy includes using CAR-T, TCR-T, TIL, CAR-NK, CAR-yST, CAR-macrophage and other engineered immune cells. In some embodiments herein, the treatment method further includes the cell therapy.

The term “EC₅₀,” as used herein, which is also termed as “half maximal effective concentration” refers to the concentration of a drug, antibody or toxicant which induces a response halfway between the baseline and maximum after a specified exposure time. In the context of the application, EC₅₀ is expressed in the unit of “nM”.

The term “isolated,” as used herein, refers to a state obtained from natural state by artificial means. If a certain “isolated” substance or component is present in nature, it is possible because its natural environment changes, or the substance is isolated from natural environment, or both.

For example, a certain un-isolated polynucleotide or polypeptide naturally exists in a certain living animal body, and the same polynucleotide or polypeptide with a high purity isolated from such a natural state is called isolated polynucleotide or polypeptide. The term “isolated” excludes neither the mixed artificial or synthesized substance nor other impure substances that do not affect the activity of the isolated substance.

The term “vector,” as used herein, refers to a nucleic acid vehicle which can have a polynucleotide inserted therein. When the vector allows for the expression of the protein encoded by the polynucleotide inserted therein, the vector is called an expression vector. The vector can have the carried genetic material elements expressed in a host cell by transformation, transduction, or transfection into the host cell. Vectors are well known by a person skilled in the art, including, but not limited to plasmids, phages, cosmids, artificial chromosome such as yeast artificial chromosome (YAC), bacterial artificial chromosome (BAC) or P1-derived artificial chromosome (PAC); phage such as X phage or M13 phage and animal virus. The animal viruses that can be used as vectors, include, but are not limited to, retrovirus (including lentivirus), adenovirus, adeno-associated virus, herpes virus (such as herpes simplex virus), pox virus, baculovirus, papillomavirus, papova virus (such as SV40). A vector may comprise multiple elements for controlling expression, including, but not limited to, a promoter sequence, a transcription initiation sequence, an enhancer sequence, a selection element and a reporter gene. In addition, a vector may comprise origin of replication.

The term “host cell,” as used herein, refers to a cellular system which can be engineered to generate proteins, protein fragments, or peptides of interest. Host cells include, without limitation, cultured cells, e.g., mammalian cultured cells derived from rodents (rats, mice, guinea pigs, or hamsters) such as CHO, BHK, NSO, SP2/0, YB2/0; or human tissues or hybridoma cells, yeast cells, and insect cells, and cells comprised within a transgenic animal or cultured tissue. The term encompasses not only the particular subject cell but also the progeny of such a cell. Because certain modifications may occur in succeeding generations due to either mutation or environmental influences, such progeny may not be identical to the parent cell, but are still included within the scope of the term “host cell”.

The term “identity,” as used herein, refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al, 1988, SIAMJ. Applied Math. 48:1073.

The term “transfection,” as used herein, refers to the process by which nucleic acids are introduced into eukaryotic cells, particularly mammalian cells. Protocols and techniques for transfection include but not limited to lipid transfection and chemical and physical methods such as electroporation. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al, 1981, Gene 13:197. In a specific embodiment of the disclosure, a vector encoding the heavy chain and/or light chain of the polypeptide complex was transfected into 293F cells.

The term “fluorescence-activated cell sorting” or “FACS,” as used herein, refers to a specialized type of flow cytometry. It provides a method for sorting a heterogeneous mixture of biological cells into two or more containers, one cell at a time, based upon the specific light scattering and fluorescent characteristics of each cell (FlowMetric. “Sorting Out Fluorescence Activated Cell Sorting”. Retrieved 2017-11-09). Instruments for carrying out FACS are known to those of skill in the art and are commercially available to the public. Examples of such instruments include FACS Star Plus, FACScan and FACSort instruments from Becton Dickinson (Foster City, Calif.) Epics C from Coulter Epics Division (Hialeah, Fla.) and MoFlo from Cytomation (Colorado Springs, Colo.).

The term “antibody-dependent cell-mediated cytotoxicity” or “ADCC,” as used herein, refers to a form of cytotoxicity in which secreted Ig bound onto Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

The term “subject” includes any human or nonhuman animal, preferably humans.

The term “cancer,” as used herein, refers to any tumor or a malignant cell growth, proliferation or metastasis-mediated, solid tumors and non-solid tumors such as leukemia, which can initiate a medical condition.

The term “autoimmune disease,” as used herein, refers to any conditions arising from an abnormal immune response to a functioning body part such as rheumatoid arthritis, systemic lupus erythematosus, inflammatory bowel disease, multiple sclerosis, which can initiate a medical condition.

The term “treatment,” “treating” or “treated,” as used herein in the context of treating a condition, pertains generally to treatment and therapy, whether of a human or an animal, in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included. For cancer, “treating” may refer to dampen or slow the tumor or malignant cell growth, proliferation, or metastasis, or some combination thereof. For tumors, “treatment” includes removal of all or part of the tumor, inhibiting or slowing tumor growth and metastasis, preventing or delaying the development of a tumor, or some combination thereof.

The term “an effective amount,” as used herein, pertains to that amount of an active compound, or a material, composition or dosage form comprising an active compound, which is effective for producing some desired therapeutic effect, commensurate with a reasonable benefit/risk ratio, when administered in accordance with a desired treatment regimen. For instance, the “an effective amount,” when used in connection with treatment of diseases or conditions such as cancers, refers to an active agent, a drug or an antibody or antigen-binding portion thereof in an amount or concentration effective to treat the said diseases or conditions.

The term “prevent,” “prevention” or “preventing,” as used herein, with reference to a certain disease condition in a mammal, refers to preventing or delaying the onset of the disease, or preventing the manifestation of clinical or subclinical symptoms thereof.

The term “pharmaceutically acceptable,” as used herein, means that the vehicle, diluent, excipient and/or salts thereof, are chemically and/or physically is compatible with other ingredients in the formulation, and the physiologically compatible with the recipient.

As used herein, the term “a pharmaceutically acceptable carrier and/or excipient” refers to a carrier and/or excipient pharmacologically and/or physiologically compatible with a subject and an active agent, which is well known in the art (see, e.g., Remington's Pharmaceutical Sciences. Edited by Gennaro AR, 19th ed. Pennsylvania: Mack Publishing Company, 1995), and includes, but is not limited to pH adjuster, surfactant, adjuvant and ionic strength enhancer. For example, the pH adjuster includes, but is not limited to, phosphate buffer; the surfactant includes, but is not limited to, cationic, anionic, or non-ionic surfactant, e.g., Tween-80; the ionic strength enhancer includes, but is not limited to, sodium chloride.

As used herein, the term “adjuvant” refers to a non-specific immunopotentiator, which can enhance immune response to an antigen or change the type of immune response in an organism when it is delivered together with the antigen to the organism or is delivered to the organism in advance. There are a variety of adjuvants, including, but not limited to, aluminium adjuvants (for example, aluminum hydroxide), Freund's adjuvants (for example, Freund's complete adjuvant and Freund's incomplete adjuvant), coryne bacterium parvum, lipopolysaccharide, cytokines, and the like. Freund's adjuvant is the most commonly used adjuvant in animal experiments now. Aluminum hydroxide adjuvant is more commonly used in clinical trials.

### IL-2 Variants

In some aspects, the present disclosure provides IL-2 variants which comprise one or more modification(s), e.g. insertion, substitution and/or deletion, compared to the wild-type IL-2 protein, such as human wild-type IL-2 protein. The mature form of human wild-type IL-2 protein is exemplified in SEQ ID NO: 1.

Residues are designated herein by the one letter amino acid code followed by the IL-2 amino acid position, e.g., F42 is the phenylalanine residue at position 42 of SEQ ID NO: 1. Substitutions are designated herein by the one letter amino acid code followed by the IL-2 amino acid position followed by the substituting one letter amino acid code., e.g., F42V is a substitution of the phenylalanine residue at position 42 of SEQ ID NO: 1 with a valine residue. By introducing a modification(s) into the amino acid sequence, the IL-2 variants as disclosed herein provide a reduced binding affinity to IL-R2a, IL-2Rβ/γc, and/or IL-2Rα/β/γc complex, resulting in reduced potency in stimulating immune cells and thus reduced toxicity in vivo.

In some embodiments, the IL-2 variant has a truncation of one or more amino acids from the C terminal of the wild-type IL-2. Said truncation of one or more amino acids may be a truncation of 1, 2, 3, 4, 5, 6, 7, 8, 9, 10, 15, 20 or more amino acid(s), as long as the IL-2 variant can retain (preferably, attenuate) the binding capacity to IL-R2a, to IL-2Rβ/γc, and/or to the combined IL-2Rα/β/γc complex. The TL-2 variant may comprise an amino acid sequence that differs from SEQ ID NO: 1 only by a C-terminal truncation. In some specific embodiments, the amino acid sequence of the IL-2 variant is selected from those as set forth in SEQ ID NOs: 2-10.

In some embodiments, the IL-2 variant has at least one amino acid substitution compared to SEQ ID NO: 1. The substitution(s) may occur at the amino acids located at the IL-2/Rα or IL-2Rβ/γc binding interface. Specifically, the substitution may occur at one or more of positions 28, 32, 38, 42, 45, 52, 72, 76, 78, 82, 110, 111, 122, 125, 127, 128 and 129 corresponding to SEQ ID NO: 1.

In some embodiments, the IL-2 variant comprises one or more substitutions at positions 25, 28, 37, 38, 41, 42, 43, 45, 52, 61, 62, 65, 68, 72, 76, 78, 82, 107, 110, 111, 122, 125, 127, 128 and 129 of the amino acid sequence as set forth in SEQ ID NO: 1. At each position, the amino acid can be substituted by any amino acid other than cysteine and the original amino acid. For example, in some embodiments, the IL-2 variant comprises a substitution at position 42 of the amino acid sequence as set forth in SEQ ID NO: 1, the original amino acid phenylalanine (F) can be substituted by any amino acid other than cysteine and phenylalanine, such as alanine (A), aspartic acid (D), glutamic acid (E), serine (S), glycine (G), histidine (H), isoleucine (I), lysine (K), leucine (L), methionine (M), asparagine (N), proline (P), glutamine (Q), arginine (R), threonine (T), valine (V), tryptophan (W), tyrosine (Y), more specifically G, V, I, or M. In some specific embodiments, the amino acid sequence of the IL-2 variant is selected from those as set forth in SEQ ID NOs: 11-13 and homologous sequences thereof with at least 95% identity.

In some embodiments, the IL-2 variant comprises a substitution at position 38 of the amino acid sequence as set forth in SEQ ID NO: 1, the original amino acid arginine (R) can be substituted by any amino acid other than cysteine and arginine, such as A, D, E, F, G, H, K, L, M, N, P, Q, I, S, T, V, W, Y, more specifically W. In some specific embodiments, the amino acid sequence of the IL-2 variant is selected from SEQ ID NO: 22 and homologous sequences thereof with at least 95% identity.

In some embodiments, the IL-2 variant comprises a substitution at position 28 of the amino acid sequence as set forth in SEQ ID NO: 1, the original amino acid isoleucine (I) can be substituted by any amino acid other than cysteine and threonine, such as A, D, E, F, G, H, T, K, M, N, P, Q, R, S, L, V, W, Y, more specifically P.

In some embodiments, the IL-2 variant comprises a substitution at position 32 of the amino acid sequence as set forth in SEQ ID NO: 1, the original amino acid lysine (K) can be substituted by any amino acid other than cysteine and threonine, such as A, D, E, F, G, H, T, I, M, N, P, Q, R, S, L, V, W, Y, more specifically D.

In some embodiments, the IL-2 variant comprises a substitution at position 52 of the amino acid sequence as set forth in SEQ ID NO: 1, the original amino acid glutamic acid (E) can be substituted by any amino acid other than cysteine and threonine, such as A, D, E, F, G, H, T, K, M, N, P, Q, R, S, L, V, W, Y, more specifically G.

In some embodiments, the IL-2 variant comprises a substitution at position 76 of the amino acid sequence as set forth in SEQ ID NO: 1, the original amino acid lysine (K) can be substituted by any amino acid other than cysteine and threonine, such as A, D, E, F, G, H, T, I, M, N, P, Q, R, S, L, V, W, Y, more specifically R.

In some embodiments, the IL-2 variant comprises a substitution at position 78 of the amino acid sequence as set forth in SEQ ID NO: 1, the original amino acid phenylalanine (F) can be substituted by any amino acid other than cysteine and threonine, such as A, D, E, K, G, H, T, I, M, N, P, Q, R, S, L, V, W, Y, more specifically G.

In some embodiments, the IL-2 variant comprises a substitution at position 82 of the amino acid sequence as set forth in SEQ ID NO: 1, the original amino acid proline (P) can be substituted by any amino acid other than cysteine and threonine, such as A, D, E, K, G, H, T, I, M, N, F, Q, R, S, L, V, W, Y, more specifically Y.

In some embodiments, the IL-2 variant comprises a substitution at position 110 of the amino acid sequence as set forth in SEQ ID NO: 1, the original amino acid glutamic acid (E) can be substituted by any amino acid other than cysteine and threonine, such as A, D, E, F, G, H, T, K, M, N, P, Q, R, S, L, V, W, Y, more specifically R, I or T.

In some embodiments, the IL-2 variant comprises a substitution at position 111 of the amino acid sequence as set forth in SEQ ID NO: 1, the original amino acid threonine (T) can be substituted by any amino acid other than cysteine and threonine, such as A, D, E, F, G, H, I, K, M, N, P, Q, R, S, L, V, W, Y, more specifically A or E.

In some embodiments, the IL-2 variant comprises a substitution at position 122 of the amino acid sequence as set forth in SEQ ID NO: 1, the original amino acid isoleucine (I) can be substituted by any amino acid other than cysteine and threonine, such as A, D, E, F, G, H, K, M, N, P, Q, R, S, T, L, V, W, Y, more specifically Y, T or V.

In some embodiments, the IL-2 variant comprises a substitution at position 125 of the amino acid sequence as set forth in SEQ ID NO: 1, the original amino acid cysteine (C) can be substituted by any amino acid other than cysteine and threonine, such as A, D, E, F, G, H, I, K, M, N, P, Q, R, S, L, V, W, Y, more specifically A.

In some embodiments, the IL-2 variant comprises a substitution at position 127 of the amino acid sequence as set forth in SEQ ID NO: 1, the original amino acid serine (S) can be substituted by any amino acid other than cysteine and threonine, such as A, D, E, F, G, H, I, K, M, N, P, Q, R, T, L, V, W, Y, more specifically A, T, D or N.

In some embodiments, the IL-2 variant comprises a substitution at position 128 of the amino acid sequence as set forth in SEQ ID NO: 1, the original amino acid isoleucine (I) can be substituted by any amino acid other than cysteine and threonine, such as A, D, E, F, G, H, K, M, N, P, Q, R, S, T, L, V, W, Y, more specifically A.

In some embodiments, the IL-2 variant comprises a substitution at position 129 of the amino acid sequence as set forth in SEQ ID NO: 1, the original amino acid isoleucine (I) can be substituted by any amino acid other than cysteine and isoleucine, such as A, D, E, F, G, H, T, K, M, N, P, Q, R, S, L, V, W, Y, more specifically L, V, A, S or T.

In some embodiments, the IL-2 variant comprises both a truncation at the C-terminal and one or more substitution(s) selected from positions 28, 32, 38, 42, 45, 52, 72, 76, 78, 82, 110, 111, 122, 125, 127, 128 and 129.

In some embodiments, the IL-2 variant comprises a C-terminal truncation of 1, 2, 3, 4, 5, 6, 7, 8, 9, or 10 amino acid(s) and a substitution at position 28, such as I28P. Specifically, the IL-2 variant may comprise a C-terminal truncation of 4 or 5 amino acid(s) and a substitution of I28P.

In some embodiments, the IL-2 variant comprises a C-terminal truncation of 1, 2, 3, 4, 5, 6, 7, 8, 9, or 10 amino acid(s) and a substitution at position 32, such as K32D. Specifically, the IL-2 variant may comprise a C-terminal truncation of 4 or 5 amino acid(s) and a substitution of K32D.

In some embodiments, the IL-2 variant comprises a C-terminal truncation of 1, 2, 3, 4, 5, 6, 7, 8, 9, or 10 amino acid(s) and a substitution at position 38, such as R38W. Specifically, the IL-2 variant may comprise a C-terminal truncation of 4 or 5 amino acid(s) and a substitution of R38W.

In some embodiments, the IL-2 variant comprises a C-terminal truncation of 1, 2, 3, 4, 5, 6, 7, 8, 9, or 10 amino acid(s) and a substitution at position 42, such as F42V, F42I, F42G, or F42M.

Specifically, the IL-2 variant may comprise a C-terminal truncation of 4 or 5 amino acid(s) and a substitution of F42V, F42I, F42G, or F42M.

In some embodiments, the IL-2 variant comprises a C-terminal truncation of 1, 2, 3, 4, 5, 6, 7, 8, 9, or 10 amino acid(s) and a substitution at position 52, such as E52G. Specifically, the IL-2 variant may comprise a C-terminal truncation of 4 or 5 amino acid(s) and a substitution of E52G.

In some embodiments, the IL-2 variant comprises a C-terminal truncation of 1, 2, 3, 4, 5, 6, 7, 8, 9, or 10 amino acid(s) and a substitution at position 76, such as K76R. Specifically, the IL-2 variant may comprise a C-terminal truncation of 4 or 5 amino acid(s) and a substitution of K76R.

In some embodiments, the IL-2 variant comprises a C-terminal truncation of 1, 2, 3, 4, 5, 6, 7, 8, 9, or 10 amino acid(s) and a substitution at position 78, such as F78G. Specifically, the IL-2 variant may comprise a C-terminal truncation of 4 or 5 amino acid(s) and a substitution of F78G.

In some embodiments, the IL-2 variant comprises a C-terminal truncation of 1, 2, 3, 4, 5, 6, 7, 8, 9, or 10 amino acid(s) and a substitution at position 82, such as P82Y. Specifically, the IL-2 variant may comprise a C-terminal truncation of 4 or 5 amino acid(s) and a substitution of P82Y.

In some embodiments, the IL-2 variant comprises a C-terminal truncation of 1, 2, 3, 4, 5, 6, 7, 8, 9, or 10 amino acid(s) and a substitution at position 110, such as E110R, E110I, or E110T.

Specifically, the IL-2 variant may comprise a C-terminal truncation of 4 or 5 amino acid(s) and a substitution of E110R, E110I, or E110T.

In some embodiments, the IL-2 variant comprises a C-terminal truncation of 1, 2, 3, 4, 5, 6, 7, 8, 9, or 10 amino acid(s) and a substitution at position 111, such as T111A or T111E.

Specifically, the IL-2 variant may comprise a C-terminal truncation of 4 or 5 amino acid(s) and a substitution of T111A or T111E.

In some embodiments, the IL-2 variant comprises a C-terminal truncation of 1, 2, 3, 4, 5, 6, 7, 8, 9, or 10 amino acid(s) and a substitution at position 122, such as I122Y, I122T, or I122V.

Specifically, the IL-2 variant may comprise a C-terminal truncation of 4 or 5 amino acid(s) and a substitution of I122Y, I122T, or I122V.

In some embodiments, the IL-2 variant comprises a C-terminal truncation of 1, 2, 3, 4, 5, 6, 7, 8, 9, or 10 amino acid(s) and a substitution at position 125, such as C125A. Specifically, the IL-2 variant may comprise a C-terminal truncation of 4 or 5 amino acid(s) and a substitution of C125A.

In some embodiments, the IL-2 variant comprises a C-terminal truncation of 1, 2, 3, 4, 5, 6, 7, 8, 9, or 10 amino acid(s) and a substitution at position 127, such as S127A, S127T, S127D or S127N. Specifically, the IL-2 variant may comprise a C-terminal truncation of 4 or 5 amino acid(s) and a substitution of S127A, S127T, S127D or S127N.

In some embodiments, the IL-2 variant comprises a C-terminal truncation of 1, 2, 3, 4, 5, 6, 7, 8, 9, or 10 amino acid(s) and a substitution at position 128, such as I128A. Specifically, the IL-2 variant may comprise a C-terminal truncation of 4 or 5 amino acid(s) and a substitution of I128A.

In some embodiments, the IL-2 variant comprises a C-terminal truncation of 1, 2, 3, 4, 5, 6, 7, 8, 9, or 10 amino acid(s) and a substitution at position 129, such as I129L, I129V, I129A, I129S or I129T. Specifically, the TL-2 variant may comprise a C-terminal truncation of 4 or 5 amino acid(s) and a substitution of I129L, I129V, I129A, I129S or I129T.

In some specific embodiments, the amino acid sequence of the IL-2 variant is selected from those as set forth in SEQ ID NOs: 14-21, 23-60 and homologous sequences thereof with at least 95% identity, for example with at least 96% identity, at least 97% identity, at least 98% identity, or at least 99% identity or higher.

In some embodiments, the IL-2 variant comprises at least two substitutions occurred at one or more of positions 28, 32, 38, 42, 45, 52, 72, 76, 78, 82, 110, 111, 122, 125, 127, 128 and 129 corresponding to SEQ ID NO: 1. In some specific embodiments, the amino acid sequence of the IL-2 variant is selected from SEQ ID NOs: 28-30, 34, 49-60 and homologous sequences thereof with at least 95% identity.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percentage of identity between two amino acid sequences can be determined by the algorithm of Needleman and Wunsch (J. Mol. Biol. 48:444-453 (1970)) which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present disclosure can further be used as a “query sequence” to perform a search against public databases to, for example, identify related sequences. Such searches can be performed using the XBLAST program (version 2.0) of Altschul, et al. (1990) J. MoI. Biol. 215:403-10. BLAST protein searches can be performed with the XBLAST program, score=50, wordlength=3 to obtain amino acid sequences homologous to the antibody molecules of the disclosure. To obtain gapped alignments for comparison purposes, Gapped BLAST can be utilized as described in Altschul et al, (1997) Nucleic Acids Res. 25(17):3389-3402. When utilizing BLAST and Gapped BLAST programs, the default parameters of the respective programs (e.g., XBLAST and NBLAST) can be used. See www.ncbi.nlm.nih.gov.

### IL-2-Comprising Fusion Proteins and Polypeptide Complexes

In some aspects, the present disclosure provides a fusion protein comprising IL-2 fused to a non-IL-2 moiety. The incorporation of non-IL-2 moiety, such as PEGs, functional analogs of PEG, lipids and long-lived serum proteins, may serve for the purpose of half-life extension. In some embodiments, the non-IL-2 moiety is an antibody Fc region. In some other embodiments, the non-IL-2 moiety is human serum albumin (HSA). In some other embodiments, the non-IL-2 moiety is an anti-HSA moiety. The generated fusion protein may be a monomer or dimer.

As will be appreciated by those in the art, both Fc and albumin fusions achieve extended half-lives not only by increasing the size of the fusion protein, but also by taking advantage of the body's natural recycling mechanism: the neonatal Fc receptor, FcRn. The pH-dependent binding of these proteins to FcRn prevents degradation of the fusion protein in the endosome. Fusion to antibody Fc can improve the solubility and stability of the fusion protein.

A major difference between Fc and HSA/anti-HSA moiety is the dimeric nature of Fc versus the monomeric structure of has/anti-HSA moiety, leading to presentation of a fused protein as a dimer or a monomer depending on the choice of fusion partner. The dimeric nature of a Fc fusion protein can produce an avidity effect where the target receptors are spaced closely enough together or are themselves dimers.

In addition, the fusion protein as disclosed herein may further attenuate affinity to IL-2Rβ/γc, to IL-2Rα, or to both and the combined IL-2Rα/β/γc complex, through steric hindrance, hydrogen bond, salt bridge, hydrophobic effects, or other intramolecular interactions that form.

In some embodiments, the present disclosure provides a polypeptide complex comprising an IL-2 moiety, an antigen-binding moiety and a full or partial hinge region plus an Fc region. The antigen-binding moiety may be a Fab, scFv, a nanobody (VHH) or a TCR.

In some embodiments, the antigen-binding portion is Fab. The IL-2 moiety may be constructed on a different chain from the Fab. Alternatively, the IL-2 moiety may be on the same heavy chain with the VH region of the Fab. Where the IL-2 moiety is on the same heavy chain with the VH region, the IL-2 moiety can be operably linked to the hinge region and Fc region (optionally via a linker), which in turn is operably linked to the VH region, i.e. the IL-2 moiety and the Fab are separated by intervening hinge region and Fc region. In some other embodiments, the IL-2 moiety and the Fab are on the same side (usually N terminal) of the Fc region.

In some embodiments, the antigen-binding portion is VHH. The IL-2 moiety may be constructed on a different chain from the VHH. Alternatively, the IL-2 moiety may be on the same heavy chain with the VH region of the VHH. Where the IL-2 moiety is on the same chain with the VHH region, the IL-2 moiety can be operably linked to the hinge region and Fc region (optionally via a linker), which in turn is operably linked to the VHH region, i.e. the IL-2 moiety and the VHH are separated by intervening hinge region and Fc region. In some other embodiments, the IL-2 moiety and the VHH are on the same side (usually N terminal) of the Fc region.

In some embodiments, the antigen-binding portion is TCR. The IL-2 moiety may be constructed on a different chain from the TCR. Alternatively, the IL-2 moiety may be on the same heavy chain with one chain (alpha or beta chain) of the TCR. Where the IL-2 moiety is on the same chain with the TCR region, the IL-2 moiety can be operably linked to the hinge region and Fc region (optionally via a linker), which in turn is operably linked to the TCR region, i.e. the IL-2 moiety and the TCR are separated by intervening hinge region and Fc region. In some other embodiments, the IL-2 moiety and the TCR are on the same side (usually N terminal) of the Fc region.

Depending on the desired biochemical (e.g. solubility and stability) and pharmacokinetics properties, different construction formats may be applied, for example, the polypeptide complex may comprise more than one IL-2 moiety or more than one antigen-binding moiety.

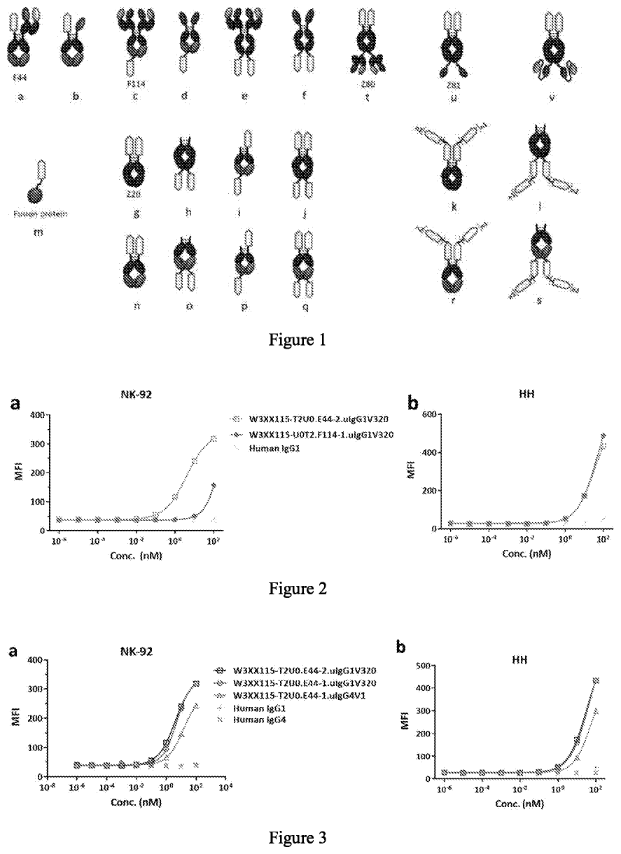

As depicted in FIG. 1(a), the polypeptide complex may comprise two heavy chains and one light chain, wherein the antigen-binding moiety is a Fab or TCR, and the first heavy chain comprises, from N-terminal to C-terminal: (a) the IL-2 moiety; (b) optionally, a linker; and (c) one chain of the hinge region and the Fc region;
the second heavy chain comprises, from N-terminal to C-terminal: (d) heavy chain of the Fab; and (e) the other chain of the hinge region and the Fc region, and
the light chain comprises light chain of the Fab or TCR.

As depicted in FIG. 1(b), the polypeptide complex may comprise two chains, wherein the antigen-binding moiety is a VHH, and the first chain comprises, from N-terminal to C-terminal: (a) the IL-2 moiety; (b) optionally, a linker; and (c) one chain of the hinge region and the Fc region;
the second chain comprises, from N-terminal to C-terminal: (d) the VHH; and (e) the other chain of the hinge region and the Fc region.

As depicted in FIG. 1(c), the polypeptide complex may comprise two heavy chains and two light chains, wherein the antigen-binding moiety is a Fab or TCR, and the first heavy chain comprises, from N-terminal to C-terminal: (a) heavy chain of the Fab or TCR; (b) one chain of the hinge region and the Fc region; (c) optionally, a linker; and (d) the IL-2 moiety, and
the second heavy chain comprises, from N-terminal to C-terminal: (e) heavy chain of the Fab or TCR; and (f) the other chain of the hinge region and the Fc region; and
the two light chains each comprising light chain of the Fab or TCR.

As depicted in FIG. 1(d), the polypeptide complex may comprise two chains, wherein the antigen-binding moiety is a VHH, and the first chain comprises, from N-terminal to C-terminal: (a) the VHH; (b) one chain of the hinge region and the Fc region; (c) optionally, a linker; and (d) the IL-2 moiety, and
the second chain comprises, from N-terminal to C-terminal: (e) the VHH; and (f) the other chain of the hinge region and the Fc region.

As depicted in FIG. 1(e), the polypeptide complex may comprise two heavy chains and two light chains, wherein the antigen-binding moiety is a Fab or TCR, and
the two heavy chains each comprising, from N-terminal to C-terminal: (a) heavy chain of the Fab or TCR; (b) one chain of the hinge region and the Fc region; (c) optionally, a linker; and (d) the IL-2 moiety, and
the two light chains each comprising light chain of the Fab or TCR.

As depicted in FIG. 1(f), the polypeptide complex may comprise two chains, wherein the antigen-binding moiety is a VHH, and
each chain comprises, from N-terminal to C-terminal: (a) the VHH; (b) one chain of the hinge region and the Fc region; (c) optionally, a linker; and (d) the IL-2 moiety.

As depicted in FIG. 1(t), the polypeptide complex may comprise two heavy chains and two light chains, wherein the antigen-binding moiety is a Fab or TCR, and
the two heavy chains each comprising, from N-terminal to C-terminal: (a) IL-2 moiety; optionally, a linker; and (b) one chain of the hinge region and the Fc region; (c) heavy chain of the Fab or TCR
the two light chains each comprising light chain of the Fab or TCR.

As depicted in FIG. 1(u), the polypeptide complex may comprise two chains, wherein the antigen-binding moiety is a VHH, and
each chain comprises, from N-terminal to C-terminal: (a) IL-2 moiety; optionally, a linker; and (b) one chain of the hinge region and the Fc region; (c) heavy chain of the VHH.

As depicted in FIG. 1(v), the polypeptide complex may comprise two chains, wherein the antigen-binding moiety is a scFv, and
each chain comprises, from N-terminal to C-terminal: (a) IL-2 moiety; optionally, a linker; and (b) one chain of the hinge region and the Fc region; (c) heavy chain of the scFv.

The target antigen for the antigen-binding moiety can be selected from checkpoint molecules such as PD-1, PD-L1, PD-L2, CTLA-4, LAG3, TIM-3, A2aR, TIGIT, VISTA, or tumor associated antigens such as HER2, and BCMA, angiogenesis related factors such as VEGF and PDGF, among others. A numerous variety of antibodies against such antigens are already developed and familiar to those skilled in the art.

The antigen-binding moiety moiety may be from or derived from antibodies which are already known, on the market, or developed de novo, such as any of the following antibodies:trastuzumab, pertuzumab, sacituzumab, abciximab, adalimumab, alefacept, alemtuzumab, basiliximab, belimumab, bezlotoxumab, bevacizuman. canakinumab, certolizumab pegol, cetuximab, daclizumab, denosumab, efalizumab, golimumab, gemtuzumab, infliximab, ipilimumab, ixekizumab, natalizumab, nivolumab, olaratumab, omalizumab, ofatumumab, palivizumab, panitumumab, pembrolizumab, rituximab, ranibizumab, tocilizumab, trastuzumab, secukinumab, and ustekinumab. The variable regions (or at least the CDR regions) of the antigen binding moieties may be same as those of the antibodies which are already known or developed de novo. By “derived from” it is meant that the variable regions are same as those in the parent antibody or have at least 80% homology (e.g. at least 85%, 90%, 95% or above) yet still retain the binding ability to the targeted antigen. For example, the variable regions from parental antibodies may be humanized, affinity matured, or glycosylation modified before being constructed into the polypeptide complexes as disclosed herein. The methods for modification of the variable regions, including CDRs and framework regions, are familiar to a person in the art.

In some exemplary embodiments, the polypeptide complex as disclosed herein comprise a Fab moiety operably linked to a Fc region and a IL-2 moiety operably linked to the C terminal of one chain of the Fc region, wherein the IL-2 moiety is consisted of wild-type IL-2 protein. Specifically, the polypeptide complex comprises a first heavy chain as set forth in SEQ ID NO: 62, a second heavy chain as set forth in SEQ ID NO: 131, and a light chain as set forth in SEQ ID NO: 134 (W3XX115-U0T2.F114-1.uIgG1V320).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of wild-type IL-2 protein. Specifically, the polypeptide complex comprises a first heavy chain as set forth in SEQ ID NO: 64 or 65, a second heavy chain as set forth in SEQ ID NO: 131 or 132, and a light chain as set forth in SEQ ID NO: 134 (W3XX115-T2U0.E44-1.uIgG4V1, W3XX115-T2U0.E44-2.uIgG1V320).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by a C-terminal truncation (e.g. truncated by 1, 2, 3, 4, 5, 6, 7, 8 or 9 amino acids) as described above. Specifically, the polypeptide complex comprises a first heavy chain as set forth in any of SEQ ID NOs: 68-76, a second heavy chain as set forth in SEQ ID NO: 132, and a light chain as set forth in SEQ ID NO: 134 (corresponding to W3XX115-T2U0.E44-3.uIgG4V1, W3XX115-T2U0.E44-4.uIgG4V1, W3XX115-T2U0.E44-5.uIgG4V1, W3XX115-T2U0.E44-6.uIgG4V1, W3XX115-T2U0.E44-7.uIgG4V1, W3XX115-T2U0.E44-8.uIgG4V1, W3XX115-T2U0.E44-9.uIgG4V1, W3XX115-T2U0.E44-10.uIgG4V1, W3XX115-T2U0.E44-11.uIgG4V1).

In some exemplary embodiments, the polypeptide complex as disclosed herein comprise a Fab moiety operably linked to one chain of a Fc region by a truncated hinge and a IL-2 moiety operably linked to the N terminal of other chain of the Fc region (optionally via a linker), wherein the IL-2 moiety is consisted of the IL-2 variant that differs from WT IL-2 by a C-terminal truncation (e.g. truncated by 1, 2, 3, 4, 5, 6, 7, 8 or 9 amino acids) as described above, and truncated hinge moiety is consisted of hinge variants that differs from WT hinge by a N-terminal truncation (e.g. truncated by 1, 2, 3, 4, 5, 6 or 7 amino acids). Specifically, the polypeptide complex comprises a first heavy chain as set forth in any of SEQ ID NOs: 89-90, a second heavy chain as set forth in SEQ ID NO: 132, and a light chain as set forth in SEQ ID NO: 134 (corresponding to W3XX115-T2U0.E44-24.uIgG4V1, W3XX115-T2U0.E44-25.uIgG4V1).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by a substitution at position F42 as described above. Specifically, the polypeptide complex comprises a first heavy chain as set forth in any of SEQ ID NOs: 77-79, a second heavy chain as set forth in SEQ ID NO: 132, and a light chain as set forth in SEQ ID NO: 134 (corresponding to W3XX115-T2U0.E44-12.uIgG4V1, W3XX115-T2U0.E44-13.uIgG4V1, W3XX115-T2U0.E44-14.uIgG4V1).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by both a C-terminal truncation (e.g. truncated by 4 or 5 amino acids) and a substitution at position R38 as described above. Specifically, the polypeptide complex comprises a first heavy chain as set forth in any of SEQ ID NO: 85, a second heavy chain as set forth in SEQ ID NO: 132, and a light chain as set forth in SEQ ID NO: 134 (corresponding to W3XX115-T2U0.E44-20.uIgG4V1).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by both a C-terminal truncation (e.g. truncated by 4 or 5 amino acids) and a substitution at position F42 as described above. Specifically, the polypeptide complex comprises a first heavy chain as set forth in any of SEQ ID NOs: 80-84, a second heavy chain as set forth in SEQ ID NO: 132, and a light chain as set forth in SEQ ID NO: 134 (corresponding to W3XX115-T2U0.E44-15.uIgG4V1, W3XX115-T2U0.E44-16.uIgG4V1, W3XX115-T2U0.E44-17.uIgG4V1, W3XX115-T2U0.E44-18.uIgG4V1, W3XX115-T2U0.E44-19.uIgG4V1).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by both a C-terminal truncation (e.g. truncated by 4 or 5 amino acids) and a substitution at position T111 as described above. Specifically, the polypeptide complex comprises a first heavy chain as set forth in any of SEQ ID NOs: 86-87, a second heavy chain as set forth in SEQ ID NO: 132, and a light chain as set forth in SEQ ID NO: 134 (corresponding to W3XX115-T2U0.E44-21.uIgG4V1, W3XX115-T2U0.E44-22.uIgG4V1).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by a substitution at position R38 as described above. Specifically, the polypeptide complex comprises a first heavy chain as set forth in SEQ ID NO: 88, a second heavy chain as set forth in SEQ ID NO: 132, and a light chain as set forth in SEQ ID NO: 134 (corresponding to W3XX115-T2U0.E44-23.uIgG4V1).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by both a C-terminal truncation (e.g. truncated by 4 amino acids) and a substitution at position 1129 as described above. Specifically, the polypeptide complex comprises a first heavy chain as set forth in any of SEQ ID NOs: 91-95, a second heavy chain as set forth in SEQ ID NO: 132, and a light chain as set forth in SEQ ID NO: 134 (corresponding to W3XX115-T2U0.E44-26.uIgG4V1, W3XX115-T2U0.E44-27.uIgG4V1, W3XX115-T2U0.E44-28.uIgG4V1, W3XX115-T2U0.E44-29.uIgG4V1, W3XX115-T2U0.E44-30.uIgG4V1).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by both a C-terminal truncation (e.g. truncated by 4 amino acids) and a substitution at position I129T and a substitution at position F42 as described above. Specifically, the polypeptide complex comprises a first heavy chain as set forth in any of SEQ ID NOs: 96-97, a second heavy chain as set forth in SEQ ID NO: 133, and a light chain as set forth in SEQ ID NO: 134 (corresponding to W3XX115-T2U0.E44-31.uIgG4V322, W3XX115-T2U0.E44-32.uIgG4V322).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by both a C-terminal truncation (e.g. truncated by 4 amino acids) and a substitution at position I129T and a substitution at position R38 as described above. Specifically, the polypeptide complex comprises a first heavy chain as set forth in any of SEQ ID NOs: 98, a second heavy chain as set forth in SEQ ID NO: 133, and a light chain as set forth in SEQ ID NO: 134 (corresponding to W3XX115-T2U0.E44-33.uIgG4V322).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by both a C-terminal truncation (e.g. truncated by 4 amino acids) and a substitution at position S127 as described above. Specifically, the polypeptide complex comprises a first heavy chain as set forth in any of SEQ ID NOs: 99-101, a second heavy chain as set forth in SEQ ID NO: 133, and a light chain as set forth in SEQ ID NO: 134 (corresponding to W3XX115-T2U0.E44-34.uIgG4V322, W3XX115-T2U0.E44-35.uIgG4V322, W3XX115-T2U0.E44-36.uIgG4V322).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by both a C-terminal truncation (e.g. truncated by 4 amino acids) and substitutions at position S127 and 1129 as described above. Specifically, the polypeptide complex comprises a first heavy chain as set forth in any of SEQ ID NOs: 102, a second heavy chain as set forth in SEQ ID NO: 133, and a light chain as set forth in SEQ ID NO: 134 (corresponding to W3XX115-T2U0.E44-37.uIgG4V322).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by both a C-terminal truncation (e.g. truncated by 4 amino acids) and a substitution at position C125 as described above. Specifically, the polypeptide complex comprises a first heavy chain as set forth in any of SEQ ID NOs: 103, a second heavy chain as set forth in SEQ ID NO: 133, and a light chain as set forth in SEQ ID NO: 134 (corresponding to W3XX115-T2U0.E44-38.uIgG4V322).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by both a C-terminal truncation (e.g. truncated by 4 amino acids) and a substitution at position 1128 as described above. Specifically, the polypeptide complex comprises a first heavy chain as set forth in any of SEQ ID NOs: 104, a second heavy chain as set forth in SEQ ID NO: 133, and a light chain as set forth in SEQ ID NO: 134 (corresponding to W3XX115-T2U0.E44-39.uIgG4V322).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by both a C-terminal truncation (e.g. truncated by 4 amino acids) and a substitution at position E110 as described above. Specifically, the polypeptide complex comprises a first heavy chain as set forth in any of SEQ ID NOs: 105-107, a second heavy chain as set forth in SEQ ID NO: 133, and a light chain as set forth in SEQ ID NO: 134 (corresponding to W3XX115-T2U0.E44-40.uIgG4V322, W3XX115-T2U0.E44-41.uIgG4V322, W3XX115-T2U0.E44-42.uIgG4V322).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by both a C-terminal truncation (e.g. truncated by 4 amino acids) and a substitution at position I122 as described above. Specifically, the polypeptide complex comprises a first heavy chain as set forth in any of SEQ ID NOs: 108-110, a second heavy chain as set forth in SEQ ID NO: 133, and a light chain as set forth in SEQ ID NO: 134 (corresponding to W3XX115-T2U0.E44-43.uIgG4V322, W3XX115-T2U0.E44-43.uIgG4V322, W3XX115-T2U0.E44-45.uIgG4V322).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by both a C-terminal truncation (e.g. truncated by 4 amino acids) and a substitution at position 128 as described above. Specifically, the polypeptide complex comprises a first heavy chain as set forth in any of SEQ ID NOs: 111, a second heavy chain as set forth in SEQ ID NO: 133, and a light chain as set forth in SEQ ID NO: 134 (corresponding to W3XX115-T2U0.E44-46.uIgG4V322).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by both a C-terminal truncation (e.g. truncated by 4 amino acids) and a substitution at position K32 as described above. Specifically, the polypeptide complex comprises a first heavy chain as set forth in any of SEQ ID NOs: 112, a second heavy chain as set forth in SEQ ID NO: 133, and a light chain as set forth in SEQ ID NO: 134 (corresponding to W3XX115-T2U0.E44-47.uIgG4V322).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by both a C-terminal truncation (e.g. truncated by 4 amino acids) and a substitution at position E52 as described above. Specifically, the polypeptide complex comprises a first heavy chain as set forth in any of SEQ ID NOs: 113, a second heavy chain as set forth in SEQ ID NO: 133, and a light chain as set forth in SEQ ID NO: 134 (corresponding to W3XX115-T2U0.E44-48.uIgG4V322).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by both a C-terminal truncation (e.g. truncated by 4 amino acids) and a substitution at position K76 as described above. Specifically, the polypeptide complex comprises a first heavy chain as set forth in any of SEQ ID NOs: 114, a second heavy chain as set forth in SEQ ID NO: 133, and a light chain as set forth in SEQ ID NO: 134 (corresponding to W3XX115-T2U0.E44-49.uIgG4V322).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by both a C-terminal truncation (e.g. truncated by 4 amino acids) and a substitution at position F78 as described above. Specifically, the polypeptide complex comprises a first heavy chain as set forth in any of SEQ ID NOs: 115, a second heavy chain as set forth in SEQ ID NO: 133, and a light chain as set forth in SEQ ID NO: 134 (corresponding to W3XX115-T2U0.E44-50.uIgG4V322).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by both a C-terminal truncation (e.g. truncated by 4 amino acids) and a substitution at position P82 as described above. Specifically, the polypeptide complex comprises a first heavy chain as set forth in any of SEQ ID NOs: 116, a second heavy chain as set forth in SEQ ID NO: 133, and a light chain as set forth in SEQ ID NO: 134 (corresponding to W3XX115-T2U0.E44-51.uIgG4V322).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by both a C-terminal truncation (e.g. truncated by 4 amino acids) and substitutions at position E110 and 1129 as described above. Specifically, the polypeptide complex comprises a first heavy chain as set forth in any of SEQ ID NOs: 117-119, a second heavy chain as set forth in SEQ ID NO: 133, and a light chain as set forth in SEQ ID NO: 134 (corresponding to W3XX115-T2U0.E44-52.uIgG4V322, W3XX115-T2U0.E44-53.uIgG4V322, W3XX115-T2U0.E44-54.uIgG4V322).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by both a C-terminal truncation (e.g. truncated by 4 amino acids) and substitutions at position I122 and I129 as described above. Specifically, the polypeptide complex comprises a first heavy chain as set forth in any of SEQ ID NOs: 120-122, a second heavy chain as set forth in SEQ ID NO: 133, and a light chain as set forth in SEQ ID NO: 134 (corresponding to W3XX115-T2U0.E44-55.uIgG4V322, W3XX115-T2U0.E44-56.uIgG4V322, W3XX115-T2U0.E44-57.uIgG4V322).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by both a C-terminal truncation (e.g. truncated by 4 amino acids) and substitutions at position 128 and 1129 as described above. Specifically, the polypeptide complex comprises a first heavy chain as set forth in any of SEQ ID NOs: 123, a second heavy chain as set forth in SEQ ID NO: 133, and a light chain as set forth in SEQ ID NO: 134 (corresponding to W3XX115-T2U0.E44-58.uIgG4V322).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by both a C-terminal truncation (e.g. truncated by 4 amino acids) and substitutions at position K32 and 1129 as described above. Specifically, the polypeptide complex comprises a first heavy chain as set forth in any of SEQ ID NOs: 124, a second heavy chain as set forth in SEQ ID NO: 133, and a light chain as set forth in SEQ ID NO: 134 (corresponding to W3XX115-T2U0.E44-59.uIgG4V322).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by both a C-terminal truncation (e.g. truncated by 4 amino acids) and substitutions at position E52 and 1129 as described above. Specifically, the polypeptide complex comprises a first heavy chain as set forth in any of SEQ ID NOs: 125, a second heavy chain as set forth in SEQ ID NO: 133, and a light chain as set forth in SEQ ID NO: 134 (corresponding to W3XX115-T2U0.E44-60.uIgG4V322).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by both a C-terminal truncation (e.g. truncated by 4 amino acids) and substitutions at position K76 and 1129 as described above. Specifically, the polypeptide complex comprises a first heavy chain as set forth in any of SEQ ID NOs: 126, a second heavy chain as set forth in SEQ ID NO: 133, and a light chain as set forth in SEQ ID NO: 134 (corresponding to W3XX115-T2U0.E44-61.uIgG4V322).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by both a C-terminal truncation (e.g. truncated by 4 amino acids) and substitutions at position F78 and 1129 as described above. Specifically, the polypeptide complex comprises a first heavy chain as set forth in any of SEQ ID NOs: 127, a second heavy chain as set forth in SEQ ID NO: 133, and a light chain as set forth in SEQ ID NO: 134 (corresponding to W3XX115-T2U0.E44-62.uIgG4V322).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by both a C-terminal truncation (e.g. truncated by 4 amino acids) and substitutions at position P82 and 1129 as described above. Specifically, the polypeptide complex comprises a first heavy chain as set forth in any of SEQ ID NOs: 128, a second heavy chain as set forth in SEQ ID NO: 133, and a light chain as set forth in SEQ ID NO: 134 (corresponding to W3XX115-T2U0.E44-63.uIgG4V322).

In some exemplary embodiments, the polypeptide complex as disclosed herein comprise a Fab moiety operably linked to one chain of a Fc region and a IL-2 moiety operably linked to the N terminal of other chain of the Fc region (optionally via a linker), wherein the IL-2 moiety is consisted of the IL-2 variant that differs from WT IL-2 by multiple substitutions (e.g. F42V/Y45A/L72G) as described above. Specifically, the polypeptide complex comprises a first heavy chain as set forth in any of SEQ ID NOs: 66, 67 and 130, a second heavy chain as set forth in any of SEQ ID NO: 131-133, and a light chain as set forth in SEQ ID NO: 134 (corresponding to W3XX115-T3U0.E44-1.uIgG4V1, W3XX115-T3U0.E44-2.uIgG1V320, W3XX115-T3U0.E44-1.uIgG4V322).

In some exemplary embodiments, the polypeptide complex as disclosed herein comprise a Fab moiety operably linked to a Fc region and a IL-2 moiety operably linked to the C terminal of one chain of the Fc region, wherein the IL-2 moiety is consisted of the IL-2 variant that differs from WT IL-2 by multiple substitutions as described above. Specifically, the heterodimeric polypeptide complex comprises a first heavy chain as set forth in SEQ ID NO: 63, a second heavy chain as set forth in SEQ ID NO: 131, and a light chain as set forth in SEQ ID NO: 134 (W3XX115-U0T3.F114-1.uIgG1V320).

### Fc Domain

The disclosure provides Fc-fusion proteins and polypeptide complexes comprising a Fc region and the human TL-2 variant as described above. The Fc domain may be a wild-type Fc or an Fc variant. A wild-type Fc may be a human IgG1, IgG2, IgG3 or IgG4 Fc. In some embodiments, the wild-type Fc is a human IgG1 Fc. The Fc variant comprises one or more amino acid residue modifications (e.g. substitutions, insertions and/or deletions) compared to the wild-type Fc (e.g, human IgG1, IgG2, IgG3 or IgG4 Fc). In some embodiments, the Fc variant comprises one or more amino acid residue modifications (e.g. substitutions, insertions and/or deletions) compared to wild-type human IgG1 Fc. In some embodiments, the Fc variant comprises one or more amino acid residue modifications (e.g. substitutions, insertions and/or deletions) compared to wild-type human IgG4 Fc.

There are many known mutations that exist for increasing or reducing ADCC, ADCP and CDC. One example of human IgG1 heavy chain mutations is LALA mutation, which prevents all effector function, i.e., essentially functions to ADCC, ADCP and CDC. The hIgG1 LALA sequence includes two mutations, L234A and L235A (EU numbering), which suppress FcgR binding. The “EU numbering system” or “EU index” is generally used when referring to a residue in an immunoglobulin heavy chain constant region (e.g., the EU index reported in Kabat et al. Sequences of Proteins of Immunological Interest (5^{th} Ed.), US Dept. of Health and Human Services, PHS, NIH, NIH Publication no. 91-3242). The “EU numbering as in Kabat” or “EU index as in Kabat” refers to the residue numbering of the human IgG1 EU antibody. Unless stated otherwise herein, references to residue numbers in the constant domain of Fc regions means residue numbering by the EU numbering system.

Said one or more amino acid modifications comprised in the Fc variant may alter the binding to one or more FcγR receptors, alter the binding to FcRn receptors, etc. In certain embodiments, the Fc domain of the fusion proteins comprise one or more amino acid substitution(s) that improves pH-dependent binding to neonatal Fc receptor (FcRn). Such a variant can have an extended pharmacokinetic half-life, as it binds to FcRn at acidic pH which allows it to escape from degradation in the lysosome and then be translocated and released out of the cell. Methods of engineering an antibody and antigen-binding fragment thereof to improve binding affinity with FcRn are well-known in the art, see, for example, Vaughn, D. et al, Structure, 6(1): 63-73, 1998; Kontermann, R. et al, Antibody Engineering, Volume 1, Chapter 27: Engineering of the Fc region for improved PK, published by Springer, 2010; Yeung, Y. et al, Cancer Research, 70: 3269-3277 (2010); and Hinton, P. et al, J. Immunology, 176:346-356 (2006).

The two chains of the Fc domain may associate together via a disulfide bond. In some embodiments, the Fc domain comprises one or more amino acid modifications (e.g. substitutions) in the interface of the Fc region to facilitate and/or promote heterodimerization. For example, the two chains of the Fc domain are engineered to comprise a “knob-into-hole” structure to promote heterodimerization, which includes introduction of a protuberance (“knob”) into a first Fc polypeptide and a cavity (“hole”) into a second Fc polypeptide, wherein the protuberance can be positioned in the cavity so as to promote interaction of the first and second Fc polypeptides to form a heterodimer or a complex. Methods of generating antibodies with these modifications are known in the art, e.g., as described in U.S. Pat. No. 5,731,168. Specifically, the Fc domain may comprise at least one “knob” (protuberance) and at least one “hole” (cavity), wherein presence of the “knob” and “hole” enhances formation of a complex or heterodimer (for more detail see WO 2005/063816). In some embodiments, the Fc domain as disclosed herein comprises a first and a second Fc polypeptide chain, wherein the first and second polypeptide each comprises one or more mutations with respect to wild type human IgG1 Fc. The IL-2 domain may be fused to one chain of the Fc domain comprising a “knob” mutation while the VH region of the antigen-binding portion is fused to the other chain comprising a “hole” mutation, or vice versa. In at least one embodiment, a “hole” mutation is Y349C, T366S, L368A, and/or Y407V, and a “knob” mutation is S354C and/or T366W.

In certain embodiments, the Fc domain of the fusion proteins comprise one or more amino acid substitution(s) that alters the antibody-dependent cellular cytotoxicity (ADCC) and/or complement dependent cytotoxicity (CDC). Certain amino acid residues at CH2 domain of the Fc region can be substituted to provide for reduced ADCC activity.

In some embodiments, the Fc domain is a IgG4 Fc variant that comprises a “FALA” mutation (i.e. F234A/L235A), which reduces the binding with Fc receptors or complement receptors. In some other embodiments, the Fc domain is a IgG4 Fc variant with truncated hinge region. In still some other embodiments, the Fc domain is a IgG4 Fc variant that comprises a S228P mutation, which may reduce IgG4 Fab-arm exchange.

In some specific embodiments, the Fc variant comprises two chains, wherein the amino acid sequence of the first chain has at least 80%, e.g 80%, 85%, 90%, 95% or more (e.g. 100%) sequence identity to wild-type human IgG1, IgG2, IgG4, or IgG4 with hinge truncation.

### Properties of the Polypeptide Complexes

The present disclosure provides affinity attenuated and potency reduced IL-2 variants and polypeptide complexes comprising the IL-2 variants. The IL-2 variants have the potential of regulated potency/toxicity, PK and PD, thus may serve as a novel immunotherapy agent with improved anti-tumor efficacy and autoimmune disease therapy.

The functionality of these IL-2 variants and IL-2 comprising polypeptide complexes may be assessed by in vitro or in vivo assays.

In some embodiments, the affinity of the IL-2 comprising polypeptide complexes to IL-2Rα or IL-2β is determined in BIAcore binding assays.

Alternatively, the binding affinity of the IL-2 comprising polypeptide complexes is determined by FACS, using cell lines that express IL-2Rβ (intermediate affinity receptor) and/or IL-2Rα (high affinity receptor), such as HH cells, NK92 and MJ20 cells.

In some embodiments, the effect of the IL-2 variants and polypeptide complexes is evaluated by quantifying a signaling pathway measured by phosphorylation of certain factors, such as STAT5 phosphorylation. STAT5 plays an important role in the maintenance of normal immune function and homeostasis, both of which are regulated by specific members of IL-2 family of cytokines.

Regulatory T cells (Treg) play a critical role to maintain immune homeostasis and self-tolerance, and is crucial in controlling the development of allergies and autoimmune diseases. Activated CD8+ T cells are very important for immune defense against tumor. IL-2 is able to induce proliferation of Treg cells and activated CD8+ T by signaling through high affinity IL-2 receptor. In some embodiments, the effect of the IL-2 variants and polypeptide complexes is evaluated by assessing T cell activation, such as CD8+ T cell or Treg cell activation.

The IL-2 comprising polypeptide complexes of the present disclosure provide at least one of the following properties:
(a) attenuated binding affinity to at least one of IL-2Rα, IL-2Rβ/γc, and the combined IL-2Rα/β/γc complex;
(b) more moderate potency in simulating immune cell proliferation, such as CD8+ T cell and NK cell proliferation;
(c) reduced potency in activating immune cells such as activated CD8+ T cells or Treg cells; and
(d) no obvious toxicity in vivo.

### Nucleic Acid Molecules Encoding the IL-2 Variants

In some aspects, the disclosure is directed to an isolated nucleic acid molecule, comprising a nucleic acid sequence encoding the IL-2 variant or the Fc fusion protein as disclosed herein.

Nucleic acids of the disclosure can be obtained using standard molecular biology techniques. The isolated nucleic acid encoding the IL-2 variant can be operatively linked to another DNA molecule encoding an Fc domain. Similarly, a nucleic acid encoding the antigen-binding portion can be operatively linked to another DNA molecule encoding an Fc domain. DNA fragments encompassing these regions can be obtained by standard PCR amplification.

Once DNA fragments encoding IL-2 moiety, the antigen-binding portion and Fc domains (or constant regions) are obtained, these DNA fragments can be further manipulated by standard recombinant DNA techniques, for example incorporated into expression vectors as is known in the art. In some embodiments, nucleic acids encoding these DNA fragments are each contained within a single expression vector, generally under different or the same promoter control. In some other embodiments, nucleic acids encoding these DNA fragments are operably linked and contained in a single expression vector under the control of the same promoter. The term “operatively linked”, as used in this context, is intended to mean that the two DNA fragments are joined such that the amino acid sequences encoded by the two DNA fragments remain in-frame.

### Host Cells

Host cells as disclosed in the present disclosure may be any cell which is suitable for expressing the fusion proteins of the present disclosure, for instance, bacterial cells, yeast, mammalian cells. Mammalian host cells for expressing the fusion proteins of the present disclosure include Chinese Hamster Ovary (CHO cells) (including dhfr CHO cells, described in Urlaub and Chasin, (1980) Proc. Natl. Acad. ScL USA 77:4216-4220, used with a DHFR selectable marker, e.g., as described in R. J. Kaufman and P. A. Sharp (1982) J. MoI. Biol. 159:601-621), NSO myeloma cells, COS cells and SP2 cells. In particular, for use with NSO myeloma cells, another expression system is the GS gene expression system disclosed in WO 87/04462, WO 89/01036 and EP 338,841. When recombinant expression vectors encoding the antibody are introduced into mammalian host cells, the fusion proteins are produced by culturing the host cells for a period of time sufficient to allow for expression of the fusion protein in the host cells or, secretion of the fusion protein into the culture medium in which the host cells are grown. The fusion proteins can be recovered from the culture medium using standard protein purification methods.

### Pharmaceutical Compositions

In some aspects, the disclosure is directed to a pharmaceutical composition comprising the fusion protein or polypeptide complex as disclosed herein and a pharmaceutically acceptable carrier. In some aspects, the disclosure is directed to a pharmaceutical composition comprising a nucleic acid molecule encoding the fusion protein or polypeptide complex as disclosed herein and a pharmaceutically acceptable carrier.

### Components of the Compositions

The pharmaceutical composition may optionally contain one or more additional pharmaceutically active ingredients, such as an antibody. The pharmaceutical compositions of the disclosure also can be administered in a combination therapy with, for example, another immune-stimulatory agent, anti-cancer agent, an antiviral agent, or a vaccine. A pharmaceutically acceptable carrier can include, for example, a pharmaceutically acceptable liquid, gel or solid carriers, an aqueous medium, a non-aqueous medium, an anti-microbial agent, isotonic agents, buffers, antioxidants, anesthetics, suspending/dispersing agent, a chelating agent, a diluent, adjuvant, excipient or a nontoxic auxiliary substance, other known in the art various combinations of components or more.

Suitable components may include, for example, antioxidants, fillers, binders, disintegrating agents, buffers, preservatives, lubricants, flavorings, thickening agents, coloring agents, emulsifiers or stabilizers such as sugars and cyclodextrin. Suitable anti-oxidants may include, for example, methionine, ascorbic acid, EDTA, sodium thiosulfate, platinum, catalase, citric acid, cysteine, mercapto glycerol, thioglycolic acid, Mercapto sorbitol, butyl methyl anisole, butylated hydroxy toluene and/or propylgalacte. As disclosed in the present disclosure, the composition may include one or more anti-oxidants such as methionine, reducing antibody or antigen binding fragment thereof that may be oxidized. The oxidation reduction may prevent or reduce a decrease in binding affinity, thereby enhancing protein stability and extended shelf life. Thus, in some embodiments, the present disclosure provides a composition comprising fusion proteins and one or more anti-oxidants such as methionine. The present disclosure further provides a variety of methods, wherein a fusion protein is mixed with one or more anti-oxidants, such as methionine, so that the fusion protein can be prevented from oxidation, to extend their shelf life and/or increased activity.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

### Administration, Formulation and Dosage

The pharmaceutical composition of the disclosure may be administered in vivo, to a subject in need thereof, by various routes, including, but not limited to, oral, intravenous, intra-arterial, subcutaneous, parenteral, intranasal, intramuscular, intracranial, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen.

Suitable formulations for enteral administration include hard or soft gelatin capsules, pills, tablets, including coated tablets, elixirs, suspensions, syrups or inhalations and controlled release forms thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Similarly, the particular dosage regimen, including dose, timing and repetition, will depend on the particular individual and that individual's medical history, as well as empirical considerations such as pharmacokinetics (e.g., half-life, clearance rate, etc.).

Frequency of administration may be determined and adjusted over the course of therapy, and is based on reducing the number of proliferative or tumorigenic cells, maintaining the reduction of such neoplastic cells, reducing the proliferation of neoplastic cells, or delaying the development of metastasis. In some embodiments, the dosage administered may be adjusted or attenuated to manage potential side effects and/or toxicity. Alternatively, sustained continuous release formulations of a subject therapeutic composition may be appropriate.

It will be appreciated by one of skill in the art that appropriate dosages can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action that achieve the desired effect without causing substantial harmful or deleterious side-effects.

In general, the polypeptide complexes of the disclosure may be administered in various ranges. These include about 100 μg/kg body weight to about 10 mg/kg body weight per dose; about 100 μg/kg body weight to about 1 mg/kg body weight per dose; about 1 μg/kg body weight to about 10 mg/kg body weight per dose. Other ranges include about 100 μg/kg body weight to about 200 μg/kg body weight per dose; about 200 μg/kg body weight to about 300 μg/kg body weight per dose; about 300 μg/kg body weight to about 400 μg/kg body weight per dose; about 400 μg/kg body weight to about 0.5 μg/kg body weight per dose; and about 0.5 mg/kg body weight to about 1 mg/kg body weight per dose. In certain embodiments, the dosage is at least about 100 μg/kg body weight, at least about 250 μg/kg body weight, at least about 750 μg/kg body weight, at least about 3 mg/kg body weight, at least about 5 mg/kg body weight, at least about 10 mg/kg body weight.

In any event, the polypeptide complexes of the disclosure are preferably administered as needed to subjects in need thereof. Determination of the frequency of administration may be made by persons skilled in the art, such as an attending physician based on considerations of the condition being treated, age of the subject being treated, severity of the condition being treated, general state of health of the subject being treated and the like.

In certain preferred embodiments, the course of treatment involving the polypeptide complexes of the present disclosure will comprise multiple doses of the selected drug product over a period of weeks or months. More specifically, the polypeptide complexes of the present disclosure may be administered once every four days, every week, every ten days, every two weeks, every three weeks, every month, every six weeks, every two months, every ten weeks or every three months. In this regard, it will be appreciated that the dosages may be altered or the interval may be adjusted based on patient response and clinical practices.

Dosages and regimens may also be determined empirically for the disclosed therapeutic compositions in individuals who have been given one or more administration(s). For example, individuals may be given incremental dosages of a therapeutic composition produced as described herein. In selected embodiments, the dosage may be gradually increased or reduced or attenuated based respectively on empirically determined or observed side effects or toxicity. To assess efficacy of the selected composition, a marker of the specific disease, disorder or condition can be followed as described previously. For cancer, these include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker (e.g., PSA for prostate cancer) or a tumorigenic antigen identified according to the methods described herein, a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of neoplastic condition, the stage of neoplastic condition, whether the neoplastic condition has begun to metastasize to other location in the individual, and the past and concurrent treatments being used.

Compatible formulations for parenteral administration (e.g., intravenous injection) will comprise the polypeptide complexes as disclosed herein in concentrations that are considered suitable for administration and can be empirically determined by those skilled in the art, for example from about 10 μg/ml to about 10 mg/ml.

### Applications of the Disclosure

The polypeptide complexes, pharmaceutical compositions and methods of the present disclosure have numerous in vitro and in vivo utilities involving, for example, enhancement of immune response. For example, these molecules can be administered to cells in culture, in vitro or ex vivo, or to human subjects, e.g., in vivo, to enhance immunity in a variety of situations. The immune response can be modulated, for instance, augmented, stimulated or up-regulated.

For instance, the subjects include human patients in need of enhancement of an immune response. The methods are particularly suitable for treating human patients having a disorder that can be treated by augmenting an immune response (e.g., the NK/T-cell mediated immune response). In a particular embodiment, the methods are particularly suitable for treatment of cancer in vivo. To achieve enhancement of immunity, the polypeptide complexes can be administered alone or in combination with another therapy. When polypeptide complexes are administered together with another agent, the two can be administered in either order or simultaneously.

For instance, the subjects include human patients in need of suppression of an immune response. The methods are particularly suitable for treating human patients having a disorder that can be treated by suppressing an immune response (e.g., the Treg mediated immune suppression). In a particular embodiment, the methods are particularly suitable for treatment of autoimmune disease in vivo. To achieve suppression of immunity, the polypeptide complexes can be administered alone or in combination with another therapy. When polypeptide complexes are administered together with another agent, the two can be administered in either order or simultaneously.

### Treatment of Disorders Including Cancers

In some aspects, the present disclosure provides a method of treating a disorder or a disease in a mammal, which comprises administering to the subject (for example, a human) in need of treatment a therapeutically effective amount of the polypeptide complexes as disclosed herein. In some aspects, the present disclosure provides a method of treating a disorder or a disease in a mammal, which comprises administering to the subject (for example, a human) in need of treatment a therapeutically effective amount of a nucleic acid molecule encoding the polypeptide complexes as disclosed herein. The disorder or disease may be a cancer.

A variety of cancers, whether malignant or benign and whether primary or secondary, may be treated or prevented with a method provided by the disclosure. The cancers may be solid cancers or hematologic malignancies. Examples of such cancers include lung cancers such as bronchogenic carcinoma (e.g., non-small cell lung cancer, squamous cell carcinoma, small cell carcinoma, large cell carcinoma, and adenocarcinoma), alveolar cell carcinoma, bronchial adenoma, chondromatous hamartoma (noncancerous), and sarcoma (cancerous); heart cancer such as myxoma, fibromas, and rhabdomyomas; bone cancers such as osteochondromas, condromas, chondroblastomas, chondromyxoid fibromas, osteoid osteomas, giant cell tumors, chondrosarcoma, multiple myeloma, osteosarcoma, fibrosarcomas, malignant fibrous histiocytomas, Ewing's tumor (Ewing's sarcoma), and reticulum cell sarcoma; brain cancer such as gliomas (e.g., glioblastoma multiforme), anaplastic astrocytomas, astrocytomas, oligodendrogliomas, medulloblastomas, chordoma, Schwannomas, ependymomas, meningiomas, pituitary adenoma, pinealoma, osteomas, hemangioblastomas, craniopharyngiomas, chordomas, germinomas, teratomas, dermoid cysts, and angiomas; cancers in digestive system such as colon cancer, leiomyoma, epidermoid carcinoma, adenocarcinoma, leiomyosarcoma, stomach adenocarcinomas, intestinal lipomas, intestinal neurofibromas, intestinal fibromas, polyps in large intestine, and colorectal cancers; liver cancers such as hepatocellular adenomas, hemangioma, hepatocellular carcinoma, fibrolamellar carcinoma, cholangiocarcinoma, hepatoblastoma, and angiosarcoma; kidney cancers such as kidney adenocarcinoma, renal cell carcinoma, hypernephroma, and transitional cell carcinoma of the renal pelvis; bladder cancers; hematological cancers such as acute lymphocytic (lymphoblastic) leukemia, acute myeloid (myelocytic, myelogenous, myeloblasts, myelomonocytic) leukemia, chronic lymphocytic leukemia (e.g., Sezary syndrome and hairy cell leukemia), chronic myelocytic (myeloid, myelogenous, granulocytic) leukemia, Hodgkin's lymphoma, non-Hodgkin's lymphoma, B cell lymphoma, mycosis fungoides, and myeloproliferative disorders (including myeloproliferative disorders such as polycythemia vera, myelofibrosis, thrombocythemia, and chronic myelocytic leukemia); skin cancers such as basal cell carcinoma, squamous cell carcinoma, melanoma, Kaposi's sarcoma, and Paget's disease; head and neck cancers; eye-related cancers such as retinoblastoma and intraoccular melanocarcinoma; male reproductive system cancers such as benign prostatic hyperplasia, prostate cancer, and testicular cancers (e.g., seminoma, teratoma, embryonal carcinoma, and choriocarcinoma); breast cancer; female reproductive system cancers such as uterine cancer (endometrial carcinoma), cervical cancer (cervical carcinoma), cancer of the ovaries (ovarian carcinoma), vulvar carcinoma, vaginal carcinoma, fallopian tube cancer, and hydatidiform mole; thyroid cancer (including papillary, follicular, anaplastic, or medullary cancer); pheochromocytomas (adrenal gland); noncancerous growths of the parathyroid glands; pancreatic cancers; and hematological cancers such as leukemias, myelomas, non-Hodgkin's lymphomas, and Hodgkin's lymphomas. In a specific embodiment, the cancer is colon cancer.

In some embodiments, examples of cancer include but not limited to B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; and post-transplant lymphoproliierative disorder (PTLD), as well as abnormal vascular proliferation associated with phakomatoses, edema (such as that associated with brain tumors), B-cell proliferative disorders, and Meigs' syndrome. More specific examples include, but are not limited to, relapsed or refractory NHL, front line low grade NHL, Stage II/IV NHL, chemotherapy resistant NHL, precursor B lymphoblastic leukemia and/or lymphoma, small lymphocytic lymphoma, B-cell chronic lymphocytic leukemia and/or prolymphocytic leukemia and/or small lymphocytic lymphoma, B-cell prolymphocytic lymphoma, immunocytoma and/or lymphoplasmacytic lymphoma, lymphoplasmacytic lymphoma, marginal zone B-cell lymphoma, splenic marginal zone lymphoma, extranodal marginal zone-MALT lymphoma, nodal marginal zone lymphoma, hairy cell leukemia, plasmacytoma and/or plasma cell myeloma, low grade/follicular lymphoma, intermediate grade/follicular NHL, mantle cell lymphoma, follicle center lymphoma (follicular), intermediate grade diffuse NHL, diffuse large B-cell lymphoma, aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) large granular lymphocytic leukemia, mycosis fungoides and/or Sezary syndrome, skin (cutaneous) lymphomas, anaplastic large cell lymphoma, angiocentric lymphoma.

In some embodiments, examples of cancer further include, but are not limited to, B-cell proliferative disorders, which further include, but are not limited to, lymphomas (e.g., B-Cell Non-Hodgkin's lymphomas (NHL)) and lymphocytic leukemias. Such lymphomas and lymphocytic leukemias include e.g. a) follicular lymphomas, b) Small Non-Cleaved Cell Lymphomas/Burkitt's lymphoma (including endemic Burkitt's lymphoma, sporadic Burkitt's lymphoma and Non-Burkitt's lymphoma), c) marginal zone lymphomas (including extranodal marginal zone B-cell lymphoma (Mucosa-associated lymphatic tissue lymphomas, MALT), nodal marginal zone B-cell lymphoma and splenic marginal zone lymphoma), d) Mantle cell lymphoma (MCL), e) Large Cell Lymphoma (including B-cell diffuse large cell lymphoma (DLCL), Diffuse Mixed Cell Lymphoma, Immunoblastic Lymphoma, Primary Mediastinal B-Cell Lymphoma, Angiocentric Lymphoma-Pulmonary B-Cell Lymphoma), f) hairy cell leukemia, g) lymphocytic lymphoma, Waldenstrom's macroglobulinemia, h) acute lymphocytic leukemia (ALL), chronic lymphocytic leukemia (CLL)/small lymphocytic lymphoma (SLL), B cell prolymphocytic leukemia, i) plasma cell neoplasms, plasma cell myeloma, multiple myeloma, plasmacytoma, and/or j) Hodgkin's disease.

In other embodiments, the disorder or disease may be autoimmune and inflammatory diseases, including but not limited to, type 1 diabetes, multiple sclerosis, lupus, rheumatoid arthritis, Systemic Lupus Erythematosus, autoimmune hepatitis, Antiphospholipid Syndrome, Wegener's Granulomatosis, Bullous Pemphigoid, Churg Strauss Syndrome, Thyroid diseases, including Graves' disease, Inflammatory bowel disease, Guillain-Barre syndrome, Psoriasis, Myasthenia gravis and Vasculitis.

Stimulation/Suppression of an Immune Response without Incurring Cytotoxicity

In some aspects, the disclosure also provides a method of enhancing (for example, stimulating) or suppressing an immune response in a subject comprising administering a polypeptide complex of the disclosure to the subject such that an immune response in the subject is enhanced while no undesired side effects are presented. For example, the subject is a mammal. In a specific embodiment, the subject is a human.

The term “enhancing an immune response” or its grammatical variations, means stimulating, evoking, increasing, improving, or augmenting any response of a mammal's immune system. The immune response may be a cellular response (i.e. cell-mediated, such as cytotoxic T lymphocyte mediated) or a humoral response (i.e. antibody mediated response), and may be a primary or secondary immune response. Examples of enhancement of immune response include increased CD4+ T, especially helper T cell activity and generation of cytolytic T cells. The enhancement of immune response can be assessed using a number of in vitro or in vivo measurements known to those skilled in the art, including, but not limited to, cytotoxic T lymphocyte assays, release of cytokines (for example IL-15 production or IFN-γ production), regression of tumors, survival of tumor bearing animals, antibody production, immune cell proliferation, expression of cell surface markers, and cytotoxicity. Typically, methods of the disclosure enhance the immune response by a mammal when compared to the immune response by an untreated mammal or a mammal not treated using the methods as disclosed herein. In one embodiment, the immune response is cytokine production, particularly IFN-γ production or IL-12 production. In another embodiment, the immune response is enhanced B cell proliferation. In contrary, “suppressing an immune response” means reducing, alleviating, decreasing or lowering the response of a mammal's immune system, which is often desired in autoimmune diseases where the immune system is overactive. The polypeptide complexes as disclosed herein may be used alone as a monotherapy, or more often, used in combination with cell immunotherapies, targeted therapies, chemical therapies or radiotherapies.

### Application in Cellular Immunotherapies and Gene Therapy

In some embodiments, IL-2 fusion proteins and variants as disclosed herein are used in cellular immunotherapy with IL-2 variants secretion. the IL-2 variants gene are constructed into therapeutic engineered immune cells, including but not limited to Tumor-Infiltrating Lymphocyte (TIL), Engineered T Cell Receptor (TCR-T), Chimeric Antigen Receptor (CAR) T Cell, CAR NK Cell, CAR Macrophage Cell, Natural Killer (NK) Cell.

In some embodiments, IL-2 fusion proteins and variants as disclosed herein are used in gene therapy. The gene coding IL-2 variants are integrated into therapeutic vectors, such as lentivirus, AAV, poxvirus, herpes zoster virus, oncolytic virus and other RNA/DNA vectors.

Combined Use with Cellular Immunotherapies

In some embodiments, the IL-2 fusion proteins as disclosed herein are used in combination with a cellular immunotherapy, also known as adoptive cell therapy. As is generally known, cellular immunotherapy is a form of treatment that uses the cells of human body's immune system to eliminate cancer. Some of these approaches involve directly isolating our own immune cells and simply expanding their numbers (e.g. performed by activating and expanding the immune cells of patient outside of the body and infused into the patient), whereas others involve genetically engineering immune cells (via gene therapy) to enhance their cancer-fighting capabilities. Cellular immunotherapies can be deployed in different ways, including but not limited to Tumor-Infiltrating Lymphocyte (TIL) therapy, Engineered T Cell Receptor (TCR-T) therapy, Chimeric Antigen Receptor (CAR) T Cell therapy, CAR NK Cell therapy, CAR Macrophage Cell therapy, Natural Killer (NK) Cell therapy.

Combined Use with Gene Therapies

In some embodiments, the IL-2 fusion proteins as disclosed herein are used in combination with a gene therapy. The gene coding IL-2 variants may be delivered to a subject by therapeutic vectors, such as viruses, including lentivirus, AAV, poxvirus, herpes zoster virus, oncolytic virus. Transfer of gene may be performed through transformation where under specific conditions the gene is directly taken up by the bacterial cells, transduction where a bacteriophage is used to transfer the genetic material and lastly transfection that involves forceful delivery of gene using either viral or non-viral vectors. The non-viral transfection methods are subdivided into physical, chemical and biological. The physical methods include electroporation, biolistic, microinjection, laser, elevated temperature, ultrasound and hydrodynamic gene transfer. The chemical methods utilize calcium-phosphate, DAE-dextran, liposomes and nanoparticles for transfection. The biological methods are increasingly using viruses for gene transfer, these viruses could either integrate within the genome of the host cell conferring a stable gene expression, whereas few other non-integrating viruses are episomal and their expression is diluted proportional to the cell division.

Combined Use with Targeted Therapies and Chemotherapies

The heterodimeric and homodimeric fusion proteins as disclosed herein may be administered as the sole active ingredient or in conjunction with, e.g. as an adjuvant to or in combination to, other drugs e.g. anti-cancer agents, immunomodulating agents or other anti-inflammatory agents, e.g. for the treatment or prevention of diseases mentioned above.

The term “anti-cancer agent” or “anti-proliferative agent” means any agent that can be used to treat a cell proliferative disorder such as cancer, and includes, but is not limited to, therapeutic antibodies, cytotoxic agents, cytostatic agents, anti-angiogenic agents, debulking agents, chemotherapeutic agents, radiotherapy and radiotherapeutic agents, targeted anti-cancer agents, BRMs, cancer vaccines, cytokines, hormone therapies, radiation therapy and anti-metastatic agents and immunotherapeutic agents.

For example, the fusion proteins as described herein may be used in combination with a wide variety of monoclonal antibodies, such as antibodies against tumor related antigens, stroma related antigens or pathways, e.g. PD-1/PD-L1, TIM-3, LAG-3, VEGF, HER2, CTLA-4; antibodies against leukocyte receptors, e.g., MHC, CD2, CD3, CD4, CD7, CD8, CD25, CD28, CD40, CD45, CD58, CD80, CD86 or their ligands; CD3 engager antibodies, NK engager antibodies; ADCC enabling anti-Tumor associated antigens; monoclonal antibodies to TNF, among others. The antibodies may include but not limited to, abciximab, adalimumab, alefacept, alemtuzumab, basiliximab, belimumab, bezlotoxumab, canakinumab, certolizumab pegol, cetuximab, daclizumab, denosumab, efalizumab, golimumab, inflectra, ipilimumab, ixekizumab, natalizumab, nivolumab, olaratumab, omalizumab, palivizumab, panitumumab, pembrolizumab, rituximab, tocilizumab, trastuzumab, secukinumab, and ustekinumab.

The antibodies may be monospecific or multi-specific. For example, the antibodies may be trispecific antibodies (TrAbs or TrioMabs), which have two variable segments for antigen binding and an Fc component to recruit immune cells. One example of TrAb is Catumaxomab for treating EpCam positive gastric and ovarian tumors. The antibodies may also be bispecific T cell engager antibodies (BiTE), such as Blinatumomab, MEHD7945A, ABT-122, XmAb5871 etc.

In some embodiments, the heterodimeric and homodimeric fusion proteins as described herein may be used in combination with immunomodulatory compounds, e.g. a recombinant binding molecule having at least a portion of the extracellular domain of CTLA4 or a mutant thereof, adhesion molecule inhibitors, e.g. LFA-1 antagonists, ICAM-1 or -3 antagonists, VCAM-4 antagonists or VLA-4 antagonists; blockers of proinflammatory cytokines, IL-1 blockers; chemokines blockers; or a chemotherapeutic agent.

For the purposes of the present disclosure a “chemotherapeutic agent” comprises a chemical compound that non-specifically decreases or inhibits the growth, proliferation, and/or survival of cancer cells (e.g., cytotoxic or cytostatic agents). Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis. In general, chemotherapeutic agents can include any chemical agent that inhibits, or is designed to inhibit, a cancerous cell or a cell likely to become cancerous or generate tumorigenic progeny (e.g., TIC). Such agents are often administered, and are often most effective, in combination, e.g., in regimens such as CHOP or FOLFIRI. Examples of anti-cancer agents that may be used in combination with the site-specific constructs of the present disclosure (either as a component of a site specific conjugate or in an unconjugated state) include, but are not limited to, e.g. paclitaxel, gemcitabine, cisplatinum, doxorubicin, 5-fiuorouracil, capecitabine, combretastatin, leucovorin etc.

For example, the heterodimeric and homodimeric fusion proteins as described herein may be used in combination with DMARD, e.g. Gold salts, sulphasalazine, antimalarias, methotrexate, D-penicillamine, azathioprine, mycophenolic acid, cyclosporine A, tacrolimus, sirolimus, minocycline, lefiunomide, glococorticoids; a calcineurin inhibitor, e.g. cyclosporin A or FK 506; a modulator of lymphocyte recirculation, e.g. FTY720 and FTY720 analogs; a mTOR inhibitor, e.g. rapamycin, 40-O-(2-hydroxyethyl)-rapamycin, CCI779, ABT578, AP23573 or TAFA-93; an ascomycin having immunosuppressive properties, e.g. ABT-281, ASM981, etc.; corticosteroids; cyclo-phosphamide; azathioprene; methotrexate; lefiunomide; mizoribine; mycophenolic acid; myco-phenolate mofetil; 15-deoxyspergualine or an immunosuppressive homologue, analogue or derivative thereof; immunosuppressive

It will be appreciated that, in selected embodiments as discussed above, such anti-cancer agents may comprise conjugates and may be associated with the disclosed heterodimeric and homodimeric polypeptide complexes prior to administration. More specifically, in certain embodiments selected anti-cancer agents will be linked to the unpaired cysteines of the engineered polypeptide complexes to provide engineered conjugates as set forth herein. Accordingly, such engineered conjugates are expressly contemplated as being within the scope of the present disclosure. In other embodiments, the disclosed anti-cancer agents will be given in combination with site-specific conjugates comprising a different therapeutic agent as set forth above.

It will be easily appreciated that, the anti-cancer agents or immunomodulating agents to be used in combination with the heterodimeric and homodimeric polypeptide complexes as disclosed herein should be compatible with the polypeptide complexes, i.e. would not reduce, disturb, or eliminate the effect of the polypeptide complexes as disclosed herein, and preferably provide a coordinating or even synergistic effect.

### As an Immune Enhancing Component/Moiety in a Multi-Specific Antibody

The polypeptide complexes as disclosed herein may be associated with a second antigen-specific binding portion to form a multi-specific antibody complex. For example, an antigen-binding portion (e.g. comprising a heavy chain variable region and a light chain variable region) may be fused with the N terminal or C terminal of the IL-2 moiety. Such multi-specific antibody complex not only have a high affinity to a targeted antigen, but also the potency of IL-2 in promoting immune cell activation.

Combined Use with Radiotherapies

The present disclosure also provides for the combination of the heterodimeric and homodimeric polypeptide complexes thereof with radiotherapy (i.e., any mechanism for inducing DNA damage locally within tumor cells such as gamma-irradiation, X-rays, UV-irradiation, microwaves, electronic emissions and the like). Combination therapy using the directed delivery of radioisotopes to tumor cells is also contemplated, and the disclosed conjugates may be used in connection with a targeted anti-cancer agent or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

As an Immune Suppressing without Incurring Cytotoxicity

In some aspects, the disclosure also provides a method of suppressing an immune response in a subject comprising administering a polypeptide complex of the disclosure to the subject such that an immune response in the subject is reduced while no undesired side effects are presented. For example, the subject is a mammal. In a specific embodiment, the subject is a human.

The term “suppressing an immune response” or its grammatical variations, means reducing any response of a mammal's immune system. The immune response may be a cellular response (i.e. cell-mediated, such as cytotoxic T lymphocyte mediated) or a humoral response (i.e. antibody mediated response), and may be a primary or secondary immune response. Examples of suppression of immune response include increased Treg cell activity and proliferation. The suppression of immune response can be assessed using a number of in vitro or in vivo measurements known to those skilled in the art, including, but not limited to, Treg lymphocyte assays, release of cytokines (for example IL-15 production or IFN-7 production), regression of autoimmune disease, survival of autoimmune animals, self-antibody production, immune cell proliferation, and cytotoxicity. Typically, methods of the disclosure enhance the immune response by a mammal when compared to the immune response by an untreated mammal or a mammal not treated using the methods as disclosed herein. In one embodiment, the immune response is cytokine production, particularly IFN-7 production or IL-17 production. In another embodiment, the immune response is suppressing B cell activity.

The polypeptide complexes as disclosed herein may be used alone as a monotherapy, or more often, used in combination with cell immunotherapies, gene therapy, targeted therapies or chemical therapies.

### Pharmaceutical Packs and Kits

Pharmaceutical packs and kits comprising one or more containers, comprising one or more doses of the polypeptide complexes are also provided. In certain embodiments, a unit dosage is provided wherein the unit dosage contains a predetermined amount of a composition comprising, for example, the polypeptide complexes, with or without one or more additional agents. For other embodiments, such a unit dosage is supplied in single-use prefilled syringe for injection. In still other embodiments, the composition contained in the unit dosage may comprise saline, sucrose, or the like; a buffer, such as phosphate, or the like; and/or be formulated within a stable and effective pH range. Alternatively, in certain embodiments, the conjugate composition may be provided as a lyophilized powder that may be reconstituted upon addition of an appropriate liquid, for example, sterile water or saline solution. In certain preferred embodiments, the composition comprises one or more substances that inhibit protein aggregation, including, but not limited to, sucrose and arginine. Any label on, or associated with, the container(s) indicates that the enclosed conjugate composition is used for treating the neoplastic disease condition of choice.

The present disclosure also provides kits for producing single-dose or multi-dose administration units of site-specific conjugates and, optionally, one or more anti-cancer agents. The kit comprises a container and a label or package insert on or associated with the container. Suitable containers include, for example, bottles, vials, syringes, etc. The containers may be formed from a variety of materials such as glass or plastic and contain a pharmaceutically effective amount of the disclosed conjugates in a conjugated or unconjugated form. In other preferred embodiments, the container(s) comprise a sterile access port (for example the container may be an intravenous solution bag or a vial having a stopper pierceable by a hypodermic injection needle). Such kits will generally contain in a suitable container a pharmaceutically acceptable formulation of the engineered conjugate and, optionally, one or more anti-cancer agents in the same or different containers. The kits may also contain other pharmaceutically acceptable formulations, either for diagnosis or combined therapy. For example, in addition to the polypeptide complexes of the disclosure such kits may contain any one or more of a range of anti-cancer agents such as chemotherapeutic or radiotherapeutic drugs; anti-angiogenic agents; anti-metastatic agents; targeted anti-cancer agents; cytotoxic agents; and/or other anti-cancer agents.

More specifically the kits may have a single container that contains the disclosed polypeptide complexes, with or without additional components, or they may have distinct containers for each desired agent. Where combined therapeutics are provided for conjugation, a single solution may be pre-mixed, either in a molar equivalent combination, or with one component in excess of the other. Alternatively, the conjugates and any optional anti-cancer agent of the kit may be maintained separately within distinct containers prior to administration to a patient. The kits may also comprise a second/third container means for containing a sterile, pharmaceutically acceptable buffer or other diluents such as bacteriostatic water for injection (BWFI), phosphate-buffered saline (PBS), Ringer's solution and dextrose solution.

When the components of the kit are provided in one or more liquid solutions, the liquid solution is preferably an aqueous solution, with a sterile aqueous or saline solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

As indicated briefly above the kits may also contain a means by which to administer the polypeptide complexes and any optional components to a patient, e.g., one or more needles, I.V. bags or syringes, or even an eye dropper, pipette, or other such like apparatus, from which the formulation may be injected or introduced into the animal or applied to a diseased area of the body.

The kits of the present disclosure will also typically include a means for containing the vials, or such like, and other component in close confinement for commercial sale, such as, e.g., injection or blow-molded plastic containers into which the desired vials and other apparatus are placed and retained.

### Sequence Listing Summary

The following Tables A-B provide a summary of the included sequences. The designation of the polypeptide complexes indicate their structure: “E44” or “F114” specifies whether the IL-2 moiety is at the N terminal or C terminal of the Fc region, “Z20” specifies two IL-2 moieties are at the N terminal of the Fc region (see FIG. 1), “uIgG1V320” refers to IgG1 Fc with LALA mutation, “uIgG4V322” refers to IgG4 Fe with FALA mutation, and “uIgG4V1” refers to IgG4 Fe with S228P and/or short hinge. The prefix “W3xx115-” can be omitted for simplicity.

SEQ ID NOs: 1-61 refer to the amino acid sequences of wild-type it-2 and IL-2 variant sequences; SEQ TD NOs: 62-130 refer to the amino acid sequences of the 1^{st} heavy chain of the polypeptide complexes, which comprises the IL-2 moiety; SEQ TD NOs: 131-133 refer to the amino acid sequences of the 2^{nd} heavy chain of the polypeptide complexes; and SEQ ID NO: 134 refers to the amino acid sequence of the light chain present when the antigen-binding moiety in the polypeptide complexes is a Fab.

TABLE A

| SEQ ID | | |
|---|---|---|
| NO: | Sequence | Description |
| 1 | APTSSSTKKTQLQLEHLLLDLQMILNGINNYKNPKLTRMLTFK | WT IL-2 |
| | FYMPKKATELKHLQCLEEELKPLEEVLNLAQSKNFHLRPRDLIS | |
| | NINVIVLELKGSETTFMCEYADETATIVEFLNRWITFCQSIISTLT | |
| 2 | APTSSSTKKTQLQLEHLLLDLQMILNGINNYKNPKLTRMLTFK | C terminal, 1 amino acid |
| | FYMPKKATELKHLQCLEEELKPLEEVLNLAQSKNFHLRPRDLIS | truncation |
| | NINVIVLELKGSETTFMCEYADETATIVEFLNRWITFCQSIISTL | |
| 3 | APTSSSTKKTQLQLEHLLLDLQMILNGINNYKNPKLTRMLTFK | C terminal, 2 amino acid |
| | FYMPKKATELKHLQCLEEELKPLEEVLNLAQSKNFHLRPRDLIS | truncation |
| | NINVIVLELKGSETTFMCEYADETATIVEFLNRWITFCQSIIST | |
| 4 | APTSSSTKKTQLQLEHLLLDLQMILNGINNYKNPKLTRMLTFK | C terminal, 3 amino acid |
| | FYMPKKATELKHLQCLEEELKPLEEVLNLAQSKNFHLRPRDLIS | truncation |
| | NINVIVLELKGSETTFMCEYADETATIVEFLNRWITFCQSIIS | |
| 5 | APTSSSTKKTQLQLEHLLLDLQMILNGINNYKNPKLTRMLTFK | C terminal, 4 amino acid |
| | FYMPKKATELKHLQCLEEELKPLEEVLNLAQSKNFHLRPRDLIS | truncation |
| | NINVIVLELKGSETTFMCEYADETATIVEFLNRWITFCQSII | |
| 6 | APTSSSTKKTQLQLEHLLLDLQMILNGINNYKNPKLTRMLTFK | C terminal, 5 amino acid |
| | FYMPKKATELKHLQCLEEELKPLEEVLNLAQSKNFHLRPRDLIS | truncation |
| | NINVIVLELKGSETTFMCEYADETATIVEFLNRWITFCQSI | |
| 7 | APTSSSTKKTQLQLEHLLLDLQMILNGINNYKNPKLTRMLTFK | C terminal, 6 amino acid |
| | FYMPKKATELKHLQCLEEELKPLEEVLNLAQSKNFHLRPRDLIS | truncation |
| | NINVIVLELKGSETTFMCEYADETATIVEFLNRWITFCQS | |
| 8 | APTSSSTKKTQLQLEHLLLDLQMILNGINNYKNPKLTRMLTFK | C terminal, 7 amino acid |
| | FYMPKKATELKHLQCLEEELKPLEEVLNLAQSKNFHLRPRDLIS | truncation |
| | NINVIVLELKGSETTFMCEYADETATIVEFLNRWITFCQ | |
| 9 | APTSSSTKKTQLQLEHLLLDLQMILNGINNYKNPKLTRMLTFK | C terminal, 8 amino acid |
| | FYMPKKATELKHLQCLEEELKPLEEVLNLAQSKNFHLRPRDLIS | truncation |
| | NINVIVLELKGSETTFMCEYADETATIVEFLNRWITFC | |
| 10 | APTSSSTKKTQLQLEHLLLDLQMILNGINNYKNPKLTRMLTFK | C terminal, 9 amino acid |
| | FYMPKKATELKHLQCLEEELKPLEEVLNLAQSKNFHLRPRDLIS | truncation |
| | NINVIVLELKGSETTFMCEYADETATIVEFLNRWITF | |
| 11 | APTSSSTKKTQLQLEHLLLDLQMILNGINNYKNPKLTRMLTGK | F42G |
| | FYMPKKATELKHLQCLEEELKPLEEVLNLAQSKNFHLRPRDLIS | |
| | NINVIVLELKGSETTFMCEYADETATIVEFLNRWITFCQSIISTLT | |
| 12 | APTSSSTKKTQLQLEHLLLDLQMILNGINNYKNPKLTRMLTVK | F42V |
| | FYMPKKATELKHLQCLEEELKPLEEVLNLAQSKNFHLRPRDLIS | |
| | NINVIVLELKGSETTFMCEYADETATIVEFLNRWITFCQSIISTLT | |
| 13 | APTSSSTKKTQLQLEHLLLDLQMILNGINNYKNPKLTRMLTIKF | F42I |
| | YMPKKATELKHLQCLEEELKPLEEVLNLAQSKNFHLRPRDLIS | |
| | NINVIVLELKGSETTFMCEYADETATIVEFLNRWITFCQSIISTLT | |
| 14 | APTSSSTKKTQLQLEHLLLDLQMILNGINNYKNPKLTRMLTVK | C terminal, 4 amino acid |
| | FYMPKKATELKHLQCLEEELKPLEEVLNLAQSKNFHLRPRDLIS | truncation and F42V |
| | NINVIVLELKGSETTFMCEYADETATIVEFLNRWITFCQSII | |
| 15 | APTSSSTKKTQLQLEHLLLDLQMILNGINNYKNPKLTRMLTIKF | C terminal, 4 amino acid |
| | YMPKKATELKHLQCLEEELKPLEEVLNLAQSKNFHLRPRDLIS | truncation and F42I |
| | NINVIVLELKGSETTFMCEYADETATIVEFLNRWITFCQSII | |
| 16 | APTSSSTKKTQLQLEHLLLDLQMILNGINNYKNPKLTRMLTVK | C terminal, 5 amino acid |
| | FYMPKKATELKHLQCLEEELKPLEEVLNLAQSKNFHLRPRDLIS | truncation and F42V |
| | NINVIVLELKGSETTFMCEYADETATIVEFLNRWITFCQSI | |
| 17 | APTSSSTKKTQLQLEHLLLDLQMILNGINNYKNPKLTRMLTIKF | C terminal, 5 amino acid |
| | YMPKKATELKHLQCLEEELKPLEEVLNLAQSKNFHLRPRDLIS | truncation and F42I |
| | NINVIVLELKGSETTFMCEYADETATIVEFLNRWITFCQSI | |
| 18 | APTSSSTKKTQLQLEHLLLDLQMILNGINNYKNPKLTRMLTM | C terminal, 4 amino acid |
| | KFYMPKKATELKHLQCLEEELKPLEEVLNLAQSKNFHLRPRDLI | truncation and F42M |
| | SNINVIVLELKGSETTFMCEYADETATIVEFLNRWITFCQSII | |
| 19 | APTSSSTKKTQLQLEHLLLDLQMILNGINNYKNPKLTWMLTFK | C terminal, 4 amino acid |
| | FYMPKKATELKHLQCLEEELKPLEEVLNLAQSKNFHLRPRDLIS | truncation and R38W |
| | NINVIVLELKGSETTFMCEYADETATIVEFLNRWITFCQSII | |
| 20 | APTSSSTKKTQLQLEHLLLDLQMILNGINNYKNPKLTRMLTFK | C terminal, 4 amino acid |
| | FYMPKKATELKHLQCLEEELKPLEEVLNLAQSKNFHLRPRDLIS | truncation and T111A |
| | NINVIVLELKGSETTFMCEYADEAATIVEFLNRWITFCQSII | |
| 21 | APTSSSTKKTQLQLEHLLLDLQMILNGINNYKNPKLTRMLTFK | C terminal, 4 amino acid |
| | FYMPKKATELKHLQCLEEELKPLEEVLNLAQSKNFHLRPRDLIS | truncation and T111E |
| | NINVIVLELKGSETTFMCEYADEEATIVEFLNRWITFCQSII | |
| 22 | APTSSSTKKTQLQLEHLLLDLQMILNGINNYKNPKLTWMLTFK | R38W |
| | FYMPKKATELKHLQCLEEELKPLEEVLNLAQSKNFHLRPRDLIS | |
| | NINVIVLELKGSETTFMCEYADETATIVEFLNRWITFCQSIISTLT | |
| 23 | APTSSSTKKTQLQLEHLLLDLQMILNGINNYKNPKLTRMLTFKFYMPKKA | C terminal, 4 amino acid |
| | TELKHLQCLEEELKPLEEVLNLAQSKNFHLRPRDLISNINVIVLELKGSETT | truncation and I129L |
| | FMCEYADETATIVEFLNRWITFCQSIL | |
| 24 | APTSSSTKKTQLQLEHLLLDLQMILNGINNYKNPKLTRMLTFKFYMPKKA | C terminal, 4 amino acid |
| | TELKHLQCLEEELKPLEEVLNLAQSKNFHLRPRDLISNINVIVLELKGSETT | truncation and I129V |
| | FMCEYADETATIVEFLNRWITFCQSIV | |
| 25 | APTSSSTKKTQLQLEHLLLDLQMILNGINNYKNPKLTRMLTFKFYMPKKA | C terminal, 4 amino acid |
| | TELKHLQCLEEELKPLEEVLNLAQSKNFHLRPRDLISNINVIVLELKGSETT | truncation and I129A |
| | FMCEYADETATIVEFLNRWITFCQSIA | |
| 26 | APTSSSTKKTQLQLEHLLLDLQMILNGINNYKNPKLTRMLTFKFYMPKKA | C terminal, 4 amino acid |
| | TELKHLQCLEEELKPLEEVLNLAQSKNFHLRPRDLISNINVIVLELKGSETT | truncation and I129S |
| | FMCEYADETATIVEFLNRWITFCQSIS | |
| 27 | APTSSSTKKTQLQLEHLLLDLQMILNGINNYKNPKLTRMLTFKFYMPKKA | C terminal, 4 amino acid |
| | TELKHLQCLEEELKPLEEVLNLAQSKNFHLRPRDLISNINVIVLELKGSETT | truncation and I129T |
| | FMCEYADETATIVEFLNRWITFCQSIT | |
| 28 | APTSSSTKKTQLQLEHLLLDLQMILNGINNYKNPKLTRMLTVK | C terminal, 4 amino acid |
| | FYMPKKATELKHLQCLEEELKPLEEVLNLAQSKNFHLRPRDLIS | truncation, I129L and |
| | NINVIVLELKGSETTFMCEYADETATIVEFLNRWITFCQSIL | F42V |
| 29 | APTSSSTKKTQLQLEHLLLDLQMILNGINNYKNPKLTRMLTIKF | C terminal, 4 amino acid |
| | YMPKKATELKHLQCLEEELKPLEEVLNLAQSKNFHLRPRDLIS | truncation,1129L, F421 |
| | NINVIVLELKGSETTFMCEYADETATIVEFLNRWITFCQSIL | |
| 30 | APTSSSTKKTQLQLEHLLLDLQMILNGINNYKNPKLTWMLTFK | C terminal, 4 amino acid |
| | FYMPKKATELKHLQCLEEELKPLEEVLNLAQSKNFHLRPRDLIS | truncation, 1129L, R38W |
| | NINVIVLELKGSETTFMCEYADETATIVEFLNRWITFCQSIL | |
| 31 | APTSSSTKKTQLQLEHLLLDLQMILNGINNYKNPKLTRMLTFK | C terminal, 4 amino acid |
| | FYMPKKATELKHLQCLEEELKPLEEVLNLAQSKNFHLRPRDLIS | truncation, S127A |
| | NINVIVLELKGSETTFMCEYADETATIVEFLNRWITFCQAII | |
| 32 | APTSSSTKKTQLQLEHLLLDLQMILNGINNYKNPKLTRMLTFK | C terminal, 4 amino acid |
| | FYMPKKATELKHLQCLEEELKPLEEVLNLAQSKNFHLRPRDLIS | truncation, S127T |
| | NINVIVLELKGSETTFMCEYADETATIVEFLNRWITFCQTII | |
| 33 | APTSSSTKKTQLQLEHLLLDLQMILNGINNYKNPKLTRMLTFK | C terminal, 4 amino acid |
| | FYMPKKATELKHLQCLEEELKPLEEVLNLAQSKNFHLRPRDLIS | truncation, S127D |
| | NINVIVLELKGSETTFMCEYADETATIVEFLNRWITFCQDII | |
| 34 | APTSSSTKKTQLQLEHLLLDLQMILNGINNYKNPKLTRMLTFK | C terminal, 4 amino acid |
| | FYMPKKATELKHLQCLEEELKPLEEVLNLAQSKNFHLRPRDLIS | truncation, S127N, I129S |
| | NINVIVLELKGSETTFMCEYADETATIVEFLNRWITFCQNIS | |
| 35 | APTSSSTKKTQLQLEHLLLDLQMILNGINNYKNPKLTRMLTFK | C terminal, 4 amino acid |
| | FYMPKKATELKHLQCLEEELKPLEEVLNLAQSKNFHLRPRDLIS | truncation, C125A |
| | NINVIVLELKGSETTFMCEYADETATIVEFLNRWITFAQSII | |
| 36 | APTSSSTKKTQLQLEHLLLDLQMILNGINNYKNPKLTRMLTFK | C terminal, 4 amino acid |
| | FYMPKKATELKHLQCLEEELKPLEEVLNLAQSKNFHLRPRDLIS | truncation, I128A |
| | NINVIVLELKGSETTFMCEYADETATIVEFLNRWITFCQSAI | |
| 37 | APTSSSTKKTQLQLEHLLLDLQMILNGINNYKNPKLTRMLTFK | C terminal truncation of 4 |
| | FYMPKKATELKHLQCLEEELKPLEEVLNLAQSKNFHLRPRDLIS | amino acids and E110R |
| | NINVIVLELKGSETTFMCEYADETATIVEFLNRWITFCQSII | substitution |
| 38 | APTSSSTKKTQLQLEHLLLDLQMILNGINNYKNPKLTRMLTFK | C terminal truncation of 4 |
| | FYMPKKATELKHLQCLEEELKPLEEVLNLAQSKNFHLRPRDLIS | amino acids and E110I |
| | NINVIVLELKGSETTFMCEYADETATIVEFLNRWITFCQSII | substitution |
| 39 | APTSSSTKKTQLQLEHLLLDLQMILNGINNYKNPKLTRMLTFK | C terminal truncation of 4 |
| | FYMPKKATELKHLQCLEEELKPLEEVLNLAQSKNFHLRPRDLIS | amino acids and E110T |
| | NINVIVLELKGSETTFMCEYADETATIVEFLNRWITFCQSII | substitution |
| 40 | APTSSSTKKTQLQLEHLLLDLQMILNGINNYKNPKLTRMLTFK | C terminal truncation of 4 |
| | FYMPKKATELKHLQCLEEELKPLEEVLNLAQSKNFHLRPRDLIS | amino acids and I122Y |
| | NINVIVLELKGSETTFMCEYADETATIVEFLNRWITFCQSII | substitution |
| 41 | APTSSSTKKTQLQLEHLLLDLQMILNGINNYKNPKLTRMLTFK | C terminal truncation of 4 |
| | FYMPKKATELKHLQCLEEELKPLEEVLNLAQSKNFHLRPRDLIS | amino acids and I122T |
| | NINVIVLELKGSETTFMCEYADETATIVEFLNRWITFCQSII | substitution |
| 42 | APTSSSTKKTQLQLEHLLLDLQMILNGINNYKNPKLTRMLTFK | C terminal truncation of 4 |
| | FYMPKKATELKHLQCLEEELKPLEEVLNLAQSKNFHLRPRDLIS | amino acids and I122V |
| | NINVIVLELKGSETTFMCEYADETATIVEFLNRWITFCQSII | substitution |
| 43 | APTSSSTKKTQLQLEHLLLDLQMILNGINNYKNPKLTRMLTFK | C terminal truncation of 4 |
| | FYMPKKATELKHLQCLEEELKPLEEVLNLAQSKNFHLRPRDLIS | amino acids and I28P |
| | NINVIVLELKGSETTFMCEYADETATIVEFLNRWITFCQSII | substitution |
| 44 | APTSSSTKKTQLQLEHLLLDLQMILNGINNYKNPKLTRMLTFK | C terminal truncation of 4 |
| | FYMPKKATELKHLQCLEEELKPLEEVLNLAQSKNFHLRPRDLIS | amino acids and K32D |
| | NINVIVLELKGSETTFMCEYADETATIVEFLNRWITFCQSII | substitution |
| 45 | APTSSSTKKTQLQLEHLLLDLQMILNGINNYKNPKLTRMLTFK | C terminal truncation of 4 |
| | FYMPKKATELKHLQCLEEELKPLEEVLNLAQSKNFHLRPRDLIS | amino acids and E52G |
| | NINVIVLELKGSETTFMCEYADETATIVEFLNRWITFCQSII | substitution |
| 46 | APTSSSTKKTQLQLEHLLLDLQMILNGINNYKNPKLTRMLTFK | C terminal truncation of 4 |
| | FYMPKKATELKHLQCLEEELKPLEEVLNLAQSKNFHLRPRDLIS | amino acids and K76R |
| | NINVIVLELKGSETTFMCEYADETATIVEFLNRWITFCQSII | substitution |
| 47 | APTSSSTKKTQLQLEHLLLDLQMILNGINNYKNPKLTRMLTFK | C terminal truncation of 4 |
| | FYMPKKATELKHLQCLEEELKPLEEVLNLAQSKNFHLRPRDLIS | amino acids and F78G |
| | NINVIVLELKGSETTFMCEYADETATIVEFLNRWITFCQSI | substitution |
| 48 | APTSSSTKKTQLQLEHLLLDLQMILNGINNYKNPKLTRMLTFK | C terminal truncation of 4 |
| | FYMPKKATELKHLQCLEEELKPLEEVLNLAQSKNFHLRPRDLIS | amino acids and P82Y |
| | NINVIVLELKGSETTFMCEYADETATIVEFLNRWITFCQSII | substitution |
| 49 | APTSSSTKKTQLQLEHLLLDLQMILNGINNYKNPKLTRMLTFK | C terminal truncation of 4 |
| | FYMPKKATELKHLQCLEEELKPLEEVLNLAQSKNFHLRPRDLIS | amino acids, E110R and |
| | NINVIVLELKGSETTFMCEYADETATIVEFLNRWITFCQSIL | I129L substitution |
| 50 | APTSSSTKKTQLQLEHLLLDLQMILNGINNYKNPKLTRMLTFK | C terminal truncation of 4 |
| | FYMPKKATELKHLQCLEEELKPLEEVLNLAQSKNFHLRPRDLIS | amino acids, E110I and |
| | NINVIVLELKGSETTFMCEYADETATIVEFLNRWITFCQSIL | I129L substitution |
| 51 | APTSSSTKKTQLQLEHLLLDLQMILNGINNYKNPKLTRMLTFK | C terminal truncation of 4 |
| | FYMPKKATELKHLQCLEEELKPLEEVLNLAQSKNFHLRPRDLIS | amino acids, E110T and |
| | NINVIVLELKGSETTFMCEYADETATIVEFLNRWITFCQSIL | I129L substitution |
| 52 | APTSSSTKKTQLQLEHLLLDLQMILNGINNYKNPKLTRMLTFK | C terminal truncation of 4 |
| | FYMPKKATELKHLQCLEEELKPLEEVLNLAQSKNFHLRPRDLIS | amino acids, I122Y and |
| | NINVIVLELKGSETTFMCEYADETATIVEFLNRWITFCQSIL | I129L substitution |
| 53 | APTSSSTKKTQLQLEHLLLDLQMILNGINNYKNPKLTRMLTFK | C terminal truncation of 4 |
| | FYMPKKATELKHLQCLEEELKPLEEVLNLAQSKNFHLRPRDLIS | amino acids, I122T and |
| | NINVIVLELKGSETTFMCEYADETATIVEFLNRWITFCQSIL | I129L substitution |
| 54 | APTSSSTKKTQLQLEHLLLDLQMILNGINNYKNPKLTRMLTFK | C terminal truncation of 4 |
| | FYMPKKATELKHLQCLEEELKPLEEVLNLAQSKNFHLRPRDLIS | amino acids, I122V and |
| | NINVIVLELKGSETTFMCEYADETATIVEFLNRWITFCQSIL | I129L substitution |
| 55 | APTSSSTKKTQLQLEHLLLDLQMILNGINNYKNPKLTRMLTFK | C terminal truncation of 4 |
| | FYMPKKATELKHLQCLEEELKPLEEVLNLAQSKNFHLRPRDLIS | amino acids, I28P and |
| | NINVIVLELKGSETTFMCEYADETATIVEFLNRWITFCQSIL | I129L substitution |
| 56 | APTSSSTKKTQLQLEHLLLDLQMILNGINNYKNPKLTRMLTFK | C terminal truncation of 4 |
| | FYMPKKATELKHLQCLEEELKPLEEVLNLAQSKNFHLRPRDLIS | amino acids, K32D and |
| | NINVIVLELKGSETTFMCEYADETATIVEFLNRWITFCQSIL | I129L substitution |
| 57 | APTSSSTKKTQLQLEHLLLDLQMILNGINNYKNPKLTRMLTFK | C terminal truncation of 4 |
| | FYMPKKATELKHLQCLEEELKPLEEVLNLAQSKNFHLRPRDLIS | amino acids, E52G and |
| | NINVIVLELKGSETTFMCEYADETATIVEFLNRWITFCQSIL | I129L substitution |
| 58 | APTSSSTKKTQLQLEHLLLDLQMILNGINNYKNPKLTRMLTFK | C terminal truncation of 4 |
| | FYMPKKATELKHLQCLEEELKPLEEVLNLAQSKNFHLRPRDLIS | amino acids, K76R and |
| | NINVIVLELKGSETTFMCEYADETATIVEFLNRWITFCQSIL | I129L substitution |
| 59 | APTSSSTKKTQLQLEHLLLDLQMILNGINNYKNPKLTRMLTFK | C terminal truncation of 4 |
| | FYMPKKATELKHLQCLEEELKPLEEVLNLAQSKNFHLRPRDLIS | amino acids, F78G and |
| | NINVIVLELKGSETTFMCEYADETATIVEFLNRWITFCQSIL | I129L substitution |
| 60 | APTSSSTKKTQLQLEHLLLDLQMILNGINNYKNPKLTRMLTFK | C terminal truncation of 4 |
| | FYMPKKATELKHLQCLEEELKPLEEVLNLAQSKNFHLRPRDLIS | amino acids, P82Y and |
| | NINVIVLELKGSETTFMCEYADETATIVEFLNRWITFCQSIL | I129L substitution |
| 61 | APTSSSTKKTQLQLEHLLLDLQMILNGINNYKNPKLTRMLTVK | F42V/Y45A/L72G |
| | FAMPKKATELKHLQCLEEELKPLEEVLNGAQSKNFHLRPRDLI | |
| | SNINVIVLELKGSETTFMCEYADETATIVEFLNRWITFCQSIISTL | |
| | T | |
| 62 | EIQLQQSGPELVKPGASVKVSCKASGYSFTDYNMYWVKQSHGESLEWI | W3XX115-U0T2.F114- |
| | GYIDPYNGGTRYNQKFKGKATLTVDKSSSTAFMHLNSLTSEDSAVYYCAR | 1.uIgG1V320, 1st heavy |
| | NGHWDGAWFAYWGQGTLVTVSAASTKGPSVFPLAPSSKSTSGGTAAL | chain |
| | GCLVKDYFPEPVTVSWNSGALTSGVHTFPAVLQSSGLYSLSSVVTVPSSSL | WT IL2 on C-terminal |
| | GTQTYICNVNHKPSNTKVDKRVEPKSCDKTHTCPPCPAPEAAGGPSVFL | |
| | FPPKPKDTLMISRTPEVTCVVVDVSHEDPEVKFNWYVDGVEVHNAKTK | |
| | PREEQYNSTYRVVSVLTVLHQDWLNGKEYKCKVSNKALPAPIEKTISKAK | |
| | GQPREPQVYTLPPCREEMTKNQVSLWCLVKGFYPSDIAVEWESNGQPE | |
| | NNYKTTPPVLDSDGSFFLYSKLTVDKSRWQQGNVFSCSVMHEALHNHY | |
| | TQKSLSLSPGGGGGSGGGGSGGGGSAPTSSSTKKTQLQLEHLLLDLQMI | |
| | LNGINNYKNPKLTRMLTFKFYMPKKATELKHLQCLEEELKPLEEVLNLAQ | |
| | SKNFHLRPRDLISNINVIVLELKGSETTFMCEYADETATIVEFLNRWITFCQ | |
| | SIISTLT | |
| 63 | EIQLQQSGPELVKPGASVKVSCKASGYSFTDYNMYWVKQSHGESLEWI | W3XX115-U0T3.F114- |
| | GYIDPYNGGTRYNQKFKGKATLTVDKSSSTAFMHLNSLTSEDSAVYYCAR | 1.uIgG1V320, 1st heavy |
| | NGHWDGAWFAYWGQGTLVTVSAASTKGPSVFPLAPSSKSTSGGTAAL | chain |
| | GCLVKDYFPEPVTVSWNSGALTSGVHTFPAVLQSSGLYSLSSVVTVPSSSL | IL2 variant on C-terminal, |
| | GTQTYICNVNHKPSNTKVDKRVEPKSCDKTHTCPPCPAPEAAGGPSVFL | abolish IL2Ralpha binding |
| | FPPKPKDTLMISRTPEVTCVVVDVSHEDPEVKFNWYVDGVEVHNAKTK | |
| | PREEQYNSTYRVVSVLTVLHQDWLNGKEYKCKVSNKALPAPIEKTISKAK | |
| | GQPREPQVYTLPPCREEMTKNQVSLWCLVKGFYPSDIAVEWESNGQPE | |
| | NNYKTTPPVLDSDGSFFLYSKLTVDKSRWQQGNVFSCSVMHEALHNHY | |
| | TQKSLSLSPGGGGGSGGGGSGGGGSAPTSSSTKKTQLQLEHLLLDLQMI | |
| | LNGINNYKNPKLTRMLTAKFAMPKKATELKHLQCLEEELKPLEEVLNGAQ | |
| | SKNFHLRPRDLISNINVIVLELKGSETTFMCEYADETATIVEFLNRWITFCQ | |
| | SIISTLT | |
| 64 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 1.uIgG4V1, 1st heavy |
| | FMCEYADETATIVEFLNRWITFCQSIISTLTESKYGPPCPPCPAPEFLGGPS | chain |
| | VFLFPPKPKDTLMISRTPEVTCVVVDVSQEDPEVQFNWYVDGVEVHNA | WT IL2 on N-terminal |
| | KTKPREEQFNSTYRVVSVLTVLHQDWLNGKEYKCKVSNKGLPSSIEKTIS | with no linker |
| | KAKGQPREPQVYTLPPCQEEMTKNQVSLWCLVKGFYPSDIAVEWESNG | |
| | QPENNYKTTPPVLDSDGSFFLYSRLTVDKSRWQEGNVFSCSVMHEALH | |
| | NHYTQKSLSLSLG | |
| 65 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 2.uIgG1V320, 1st heavy |
| | FMCEYADETATIVEFLNRWITFCQSIISTLTGGGGSGGGGSGGGGSEPKS | chain |
| | SDKTHTCPPCPAPEAAGGPSVFLFPPKPKDTLMISRTPEVTCVVVDVSHE | WT IL2 on N-terminal |
| | DPEVKFNWYVDGVEVHNAKTKPREEQYNSTYRVVSVLTVLHQDWLNG | with (G4S)3 linker, hIgG1 |
| | KEYKCKVSNKALPAPIEKTISKAKGQPREPQVYTLPPCREEMTKNQVSLW | Fc with LALA mutation |
| | CLVKGFYPSDIAVEWESNGQPENNYKTTPPVLDSDGSFFLYSKLTVDKSR | |
| | WQQGNVFSCSVMHEALHNHYTQKSLSLSPG | |
| 66 | APTSSSTKKTQLQLEHLLLDLQMILNGINNYKNPKLTRMLTAKFAMPKKA | W3XX115-T3U0.E44- |
| | TELKHLQCLEEELKPLEEVLNGAQSKNFHLRPRDLISNINVIVLELKGSETT | 1.uIgG4V1, 1st heavy |
| | FMCEYADETATIVEFLNRWITFCQSIISTLTESKYGPPCPPCPAPEFLGGPS | chain |
| | VFLFPPKPKDTLMISRTPEVTCVVVDVSQEDPEVQFNWYVDGVEVHNA | IL2 variant on N-terminal |
| | KTKPREEQFNSTYRVVSVLTVLHQDWLNGKEYKCKVSNKGLPSSIEKTIS | with no linker |
| | KAKGQPREPQVYTLPPCQEEMTKNQVSLWCLVKGFYPSDIAVEWESNG | |
| | QPENNYKTTPPVLDSDGSFFLYSRLTVDKSRWQEGNVFSCSVMHEALH | |
| | NHYTQKSLSLSLG | |
| 67 | APTSSSTKKTQLQLEHLLLDLQMILNGINNYKNPKLTRMLTAKFAMPKKA | W3XX115-T3U0.E44- |
| | TELKHLQCLEEELKPLEEVLNGAQSKNFHLRPRDLISNINVIVLELKGSETT | 2.uIgG1V320, 1st heavy |
| | FMCEYADETATIVEFLNRWITFCQSIISTLTGGGGGGGGSGGGGSEPKS | chain |
| | SDKTHTCPPCPAPEAAGGPSVFLFPPKPKDTLMISRTPEVTCVVVDVSHE | IL2 variant on N-terminal |
| | DPEVKFNWYVDGVEVHNAKTKPREEQYNSTYRVVSVLTVLHQDWLNG | with (G4S)3 linker |
| | KEYKCKVSNKALPAPIEKTISKAKGQPREPQVYTLPPCREEMTKNQVSLW | |
| | CLVKGFYPSDIAVEWESNGQPENNYKTTPPVLDSDGSFFLYSKLTVDKSR | |
| | WQQGNVFSCSVMHEALHNHYTQKSLSLSPG | |
| 68 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 3.uIgG4V1, 1st heavy |
| | FMCEYADETATIVEFLNRWITFCQSIISTLESKYGPPCPPCPAPEFLGGPSV | chain |
| | FLFPPKPKDTLMISRTPEVTCVVVDVSQEDPEVQFNWYVDGVEVHNAK | WT IL2 with C-terminal |
| | TKPREEQFNSTYRVVSVLTVLHQDWLNGKEYKCKVSNKGLPSSIEKTISK | one amino acids deletion, |
| | AKGQPREPQVYTLPPCQEEMTKNQVSLWCLVKGFYPSDIAVEWESNG | fuse on N-terminal with no |
| | QPENNYKTTPPVLDSDGSFFLYSRLTVDKSRWQEGNVFSCSVMHEALH | linker |
| | NHYTQKSLSLSLG | |
| 69 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 4.uIgG4V1, 1st heavy |
| | FMCEYADETATIVEFLNRWITFCQSIISTESKYGPPCPPCPAPEFLGGPSVF | chain |
| | LFPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | two amino acids deletion, |
| | KGQPREPQVYTLPPCQEEMTKNQVSLWCLVKGFYPSDIAVEWESNGQP | fuse on N-terminal with no |
| | ENNYKTTPPVLDSDGSFFLYSRLTVDKSRWQEGNVFSCSVMHEALHNH | linker |
| | YTQKSLSLSLG | |
| 70 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 5.uIgG4V1, 1st heavy |
| | FMCEYADETATIVEFLNRWITFCQSIISESKYGPPCPPCPAPEFLGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | three amino acids deletion, |
| | KGQPREPQVYTLPPCQEEMTKNQVSLWCLVKGFYPSDIAVEWESNGQP | fuse on N-terminal with no |
| | ENNYKTTPPVLDSDGSFFLYSRLTVDKSRWQEGNVFSCSVMHEALHNH | linker |
| | YTQKSLSLSLG | |
| 71 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 6.uIgG4V1, 1st heavy |
| | FMCEYADETATIVEFLNRWITFCQSIIESKYGPPCPPCPAPEFLGGPSVFLF | chain |
| | PPKPKDTLMISRTPEVTCVVVDVSQEDPEVQFNWYVDGVEVHNAKTK | WT IL2 with C-terminal |
| | PREEQFNSTYRVVSVLTVLHQDWLNGKEYKCKVSNKGLPSSIEKTISKAK | four amino acids deletion, |
| | GQPREPQVYTLPPCQEEMTKNQVSLWCLVKGFYPSDIAVEWESNGQP | fuse on N-terminal with no |
| | ENNYKTTPPVLDSDGSFFLYSRLTVDKSRWQEGNVFSCSVMHEALHNH | linker |
| | YTQKSLSLSLG | |
| 72 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 7.uIgG4V1, 1st heavy |
| | FMCEYADETATIVEFLNRWITFCQSIESKYGPPCPPCPAPEFLGGPSVFLF | chain |
| | PPKPKDTLMISRTPEVTCVVVDVSQEDPEVQFNWYVDGVEVHNAKTK | WT IL2 with C-terminal |
| | PREEQFNSTYRVVSVLTVLHQDWLNGKEYKCKVSNKGLPSSIEKTISKAK | five amino acids deletion, |
| | GQPREPQVYTLPPCQEEMTKNQVSLWCLVKGFYPSDIAVEWESNGQP | fuse on N-terminal with no |
| | ENNYKTTPPVLDSDGSFFLYSRLTVDKSRWQEGNVFSCSVMHEALHNH | linker |
| | YTQKSLSLSLG | |
| 73 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 8.uIgG4V1, 1st heavy |
| | FMCEYADETATIVEFLNRWITFCQSESKYGPPCPPCPAPEFLGGPSVFLFP | chain |
| | PKPKDTLMISRTPEVTCVVVDVSQEDPEVQFNWYVDGVEVHNAKTKP | WT IL2 with C-terminal |
| | REEQFNSTYRVVSVLTVLHQDWLNGKEYKCKVSNKGLPSSIEKTISKAKG | six amino acids deletion, |
| | QPREPQVYTLPPCQEEMTKNQVSLWCLVKGFYPSDIAVEWESNGQPE | fuse on N-terminal with no |
| | NNYKTTPPVLDSDGSFFLYSRLTVDKSRWQEGNVFSCSVMHEALHNHY | linker |
| | TQKSLSLSLG | |
| 74 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 9.uIgG4V1, 1st heavy |
| | FMCEYADETATIVEFLNRWITFCQESKYGPPCPPCPAPEFLGGPSVFLFPP | chain |
| | KPKDTLMISRTPEVTCVVVDVSQEDPEVQFNWYVDGVEVHNAKTKPR | WT IL2 with C-terminal |
| | EEQFNSTYRVVSVLTVLHQDWLNGKEYKCKVSNKGLPSSIEKTISKAKGQ | seven amino acids deletion, |
| | PREPQVYTLPPCQEEMTKNQVSLWCLVKGFYPSDIAVEWESNGQPENN | fuse on N-terminal with no |
| | YKTTPPVLDSDGSFFLYSRLTVDKSRWQEGNVFSCSVMHEALHNHYTQ | linker |
| | KSLSLSLG | |
| 75 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 10.uIgG4V1, 1st heavy |
| | FMCEYADETATIVEFLNRWITFCESKYGPPCPPCPAPEFLGGPSVFLFPPK | chain |
| | PKDTLMISRTPEVTCVVVDVSQEDPEVQFNWYVDGVEVHNAKTKPRE | WT IL2 with C-terminal |
| | EQFNSTYRVVSVLTVLHQDWLNGKEYKCKVSNKGLPSSIEKTISKAKGQP | eight amino acids deletion, |
| | REPQVYTLPPCQEEMTKNQVSLWCLVKGFYPSDIAVEWESNGQPENNY | fuse on N-terminal with no |
| | KTTPPVLDSDGSFFLYSRLTVDKSRWQEGNVFSCSVMHEALHNHYTQKS | linker |
| | LSLSLG | |
| 76 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 11.uIgG4V1, 1st heavy |
| | FMCEYADETATIVEFLNRWITFESKYGPPCPPCPAPEFLGGPSVFLFPPKP | chain |
| | KDTLMISRTPEVTCVVVDVSQEDPEVQFNWYVDGVEVHNAKTKPREE | WT IL2 with C-terminal |
| | QFNSTYRVVSVLTVLHQDWLNGKEYKCKVSNKGLPSSIEKTISKAKGQPR | nine amino acids deletion, |
| | EPQVYTLPPCQEEMTKNQVSLWCLVKGFYPSDIAVEWESNGQPENNYK | fuse on N-terminal with no |
| | TTPPVLDSDGSFFLYSRLTVDKSRWQEGNVFSCSVMHEALHNHYTQKSL | linker |
| | SLSLG | |
| 77 | APTSSSTKKTQLQLEHLLLDLQMILNGINNYKNPKLTRMLTGKFYMPKKA | W3XX115-T2U0.E44- |
| | TELKHLQCLEEELKPLEEVLNLAQSKNFHLRPRDLISNINVIVLELKGSETT | 12.uIgG4V1, 1st heavy |
| | FMCEYADETATIVEFLNRWITFCQSIISTLTESKYGPPCPPCPAPEFLGGPS | chain |
| | VFLFPPKPKDTLMISRTPEVTCVVVDVSQEDPEVQFNWYVDGVEVHNA | WT IL2 with F42G, fuse |
| | KTKPREEQFNSTYRVVSVLTVLHQDWLNGKEYKCKVSNKGLPSSIEKTIS | on N-terminal with no |
| | KAKGQPREPQVYTLPPCQEEMTKNQVSLWCLVKGFYPSDIAVEWESNG | linker |
| | QPENNYKTTPPVLDSDGSFFLYSRLTVDKSRWQEGNVFSCSVMHEALH | |
| | NHYTQKSLSLSLG | |
| 78 | APTSSSTKKTQLQLEHLLLDLQMILNGINNYKNPKLTRMLTVKFYMPKKA | W3XX115-T2U0.E44- |
| | TELKHLQCLEEELKPLEEVLNLAQSKNFHLRPRDLISNINVIVLELKGSETT | 13.uIgG4V1, 1st heavy |
| | FMCEYADETATIVEFLNRWITFCQSIISTLTESKYGPPCPPCPAPEFLGGPS | chain |
| | VFLFPPKPKDTLMISRTPEVTCVVVDVSQEDPEVQFNWYVDGVEVHNA | WT IL2 with F42V, fuse on |
| | KTKPREEQFNSTYRVVSVLTVLHQDWLNGKEYKCKVSNKGLPSSIEKTIS | N-terminal with no linker |
| | KAKGQPREPQVYTLPPCQEEMTKNQVSLWCLVKGFYPSDIAVEWESNG | |
| | QPENNYKTTPPVLDSDGSFFLYSRLTVDKSRWQEGNVFSCSVMHEALH | |
| | NHYTQKSLSLSLG | |
| 79 | APTSSSTKKTQLQLEHLLLDLQMILNGINNYKNPKLTRMLTIKFYMPKKA | W3XX115-T2U0.E44- |
| | TELKHLQCLEEELKPLEEVLNLAQSKNFHLRPRDLISNINVIVLELKGSETT | 14.uIgG4V1, 1st heavy |
| | FMCEYADETATIVEFLNRWITFCQSIISTLTESKYGPPCPPCPAPEFLGGPS | chain |
| | VFLFPPKPKDTLMISRTPEVTCVVVDVSQEDPEVQFNWYVDGVEVHNA | WT IL2 with F42I, fuse on |
| | KTKPREEQFNSTYRVVSVLTVLHQDWLNGKEYKCKVSNKGLPSSIEKTIS | N-terminal with no linker |
| | KAKGQPREPQVYTLPPCQEEMTKNQVSLWCLVKGFYPSDIAVEWESNG | |
| | QPENNYKTTPPVLDSDGSFFLYSRLTVDKSRWQEGNVFSCSVMHEALH | |
| | NHYTQKSLSLSLG | |
| 80 | APTSSSTKKTQLQLEHLLLDLQMILNGINNYKNPKLTRMLTVKFYMPKKA | W3XX115-T2U0.E44- |
| | TELKHLQCLEEELKPLEEVLNLAQSKNFHLRPRDLISNINVIVLELKGSETT | 15.uIgG4V1, 1st heavy |
| | FMCEYADETATIVEFLNRWITFCQSIIESKYGPPCPPCPAPEFLGGPSVFLF | chain |
| | PPKPKDTLMISRTPEVTCVVVDVSQEDPEVQFNWYVDGVEVHNAKTK | WT IL2 with C-terminal |
| | PREEQFNSTYRVVSVLTVLHQDWLNGKEYKCKVSNKGLPSSIEKTISKAK | four amino acids deletion |
| | GQPREPQVYTLPPCQEEMTKNQVSLWCLVKGFYPSDIAVEWESNGQP | and F42V, fuse on N- |
| | ENNYKTTPPVLDSDGSFFLYSRLTVDKSRWQEGNVFSCSVMHEALHNH | terminal with no linker |
| | YTQKSLSLSLG | |
| 81 | APTSSSTKKTQLQLEHLLLDLQMILNGINNYKNPKLTRMLTIKFYMPKKA | W3XX115-T2U0.E44- |
| | TELKHLQCLEEELKPLEEVLNLAQSKNFHLRPRDLISNINVIVLELKGSETT | 16.uIgG4V1, 1st heavy |
| | FMCEYADETATIVEFLNRWITFCQSIIESKYGPPCPPCPAPEFLGGPSVFLF | chain |
| | PPKPKDTLMISRTPEVTCVVVDVSQEDPEVQFNWYVDGVEVHNAKTK | WT IL2 with C-terminal |
| | PREEQFNSTYRVVSVLTVLHQDWLNGKEYKCKVSNKGLPSSIEKTISKAK | four amino acids deletion |
| | GQPREPQVYTLPPCQEEMTKNQVSLWCLVKGFYPSDIAVEWESNGQP | and F421, fuse on N- |
| | ENNYKTTPPVLDSDGSFFLYSRLTVDKSRWQEGNVFSCSVMHEALHNH | terminal with no linker |
| | YTQKSLSLSLG | |
| 82 | APTSSSTKKTQLQLEHLLLDLQMILNGINNYKNPKLTRMLTVKFYMPKKA | W3XX115-T2U0.E44- |
| | TELKHLQCLEEELKPLEEVLNLAQSKNFHLRPRDLISNINVIVLELKGSETT | 17.uIgG4V1, 1st heavy |
| | FMCEYADETATIVEFLNRWITFCQSIESKYGPPCPPCPAPEFLGGPSVFLF | chain |
| | PPKPKDTLMISRTPEVTCVVVDVSQEDPEVQFNWYVDGVEVHNAKTK | WT IL2 with C-terminal |
| | PREEQFNSTYRVVSVLTVLHQDWLNGKEYKCKVSNKGLPSSIEKTISKAK | five amino acids deletion |
| | GQPREPQVYTLPPCQEEMTKNQVSLWCLVKGFYPSDIAVEWESNGQP | and F42V, fuse on N- |
| | ENNYKTTPPVLDSDGSFFLYSRLTVDKSRWQEGNVFSCSVMHEALHNH | terminal with no linker |
| | YTQKSLSLSLG | |
| 83 | APTSSSTKKTQLQLEHLLLDLQMILNGINNYKNPKLTRMLTIKFYMPKKA | W3XX115-T2U0.E44- |
| | TELKHLQCLEEELKPLEEVLNLAQSKNFHLRPRDLISNINVIVLELKGSETT | 18.uIgG4V1, 1st heavy |
| | FMCEYADETATIVEFLNRWITFCQSIESKYGPPCPPCPAPEFLGGPSVFLF | chain |
| | PPKPKDTLMISRTPEVTCVVVDVSQEDPEVQFNWYVDGVEVHNAKTK | WT IL2 with C-terminal |
| | PREEQFNSTYRVVSVLTVLHQDWLNGKEYKCKVSNKGLPSSIEKTISKAK | five amino acids deletion |
| | GQPREPQVYTLPPCQEEMTKNQVSLWCLVKGFYPSDIAVEWESNGQP | and F421, fuse on N- |
| | ENNYKTTPPVLDSDGSFFLYSRLTVDKSRWQEGNVFSCSVMHEALHNH | terminal with no linker |
| | YTQKSLSLSLG | |
| 84 | APTSSSTKKTQLQLEHLLLDLQMILNGINNYKNPKLTRMLTMKFYMPKK | W3XX115-T2U0.E44- |
| | ATELKHLQCLEEELKPLEEVLNLAQSKNFHLRPRDLISNINVIVLELKGSET | 19.uIgG4V1, 1st heavy |
| | TFMCEYADETATIVEFLNRWITFCQSIIESKYGPPCPPCPAPEFLGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion |
| | KGQPREPQVYTLPPCQEEMTKNQVSLWCLVKGFYPSDIAVEWESNGQP | and F42M, fuse on N- |
| | ENNYKTTPPVLDSDGSFFLYSRLTVDKSRWQEGNVFSCSVMHEALHNH | terminal with no linker |
| | YTQKSLSLSLG | |
| 85 | APTSSSTKKTQLQLEHLLLDLQMILNGINNYKNPKLTWMLTFKFYMPKK | W3XX115-T2U0.E44- |
| | ATELKHLQCLEEELKPLEEVLNLAQSKNFHLRPRDLISNINVIVLELKGSET | 20.uIgG4V1, 1st heavy |
| | TFMCEYADETATIVEFLNRWITFCQSIIESKYGPPCPPCPAPEFLGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion |
| | KGQPREPQVYTLPPCQEEMTKNQVSLWCLVKGFYPSDIAVEWESNGQP | and R38W, fuse on N- |
| | ENNYKTTPPVLDSDGSFFLYSRLTVDKSRWQEGNVFSCSVMHEALHNH | terminal with no linker |
| | YTQKSLSLSLG | |
| 86 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 21.uIgG4V1, 1st heavy |
| | FMCEYADEAATIVEFLNRWITFCQSIIESKYGPPCPPCPAPEFLGGPSVFLF | chain |
| | PPKPKDTLMISRTPEVTCVVVDVSQEDPEVQFNWYVDGVEVHNAKTK | WT IL2 with C-terminal |
| | PREEQFNSTYRVVSVLTVLHQDWLNGKEYKCKVSNKGLPSSIEKTISKAK | four amino acids deletion |
| | GQPREPQVYTLPPCQEEMTKNQVSLWCLVKGFYPSDIAVEWESNGQP | and T111A, fuse on N- |
| | ENNYKTTPPVLDSDGSFFLYSRLTVDKSRWQEGNVFSCSVMHEALHNH | terminal with no linker |
| | YTQKSLSLSLG | |
| 87 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 22.uIgG4V1, 1st heavy |
| | FMCEYADEEATIVEFLNRWITFCQSIIESKYGPPCPPCPAPEFLGGPSVFLF | chain |
| | PPKPKDTLMISRTPEVTCVVVDVSQEDPEVQFNWYVDGVEVHNAKTK | WT IL2 with C-terminal |
| | PREEQFNSTYRVVSVLTVLHQDWLNGKEYKCKVSNKGLPSSIEKTISKAK | four amino acids deletion |
| | GQPREPQVYTLPPCQEEMTKNQVSLWCLVKGFYPSDIAVEWESNGQP | and T111E, fuse on N- |
| | ENNYKTTPPVLDSDGSFFLYSRLTVDKSRWQEGNVFSCSVMHEALHNH | terminal with no linker |
| | YTQKSLSLSLG | |
| 88 | APTSSSTKKTQLQLEHLLLDLQMILNGINNYKNPKLTWMLTFKFYMPKK | W3XX115-T2U0.E44- |
| | ATELKHLQCLEEELKPLEEVLNLAQSKNFHLRPRDLISNINVIVLELKGSET | 23.uIgG4V1, 1st heavy |
| | TFMCEYADETATIVEFLNRWITFCQSIISTLTESKYGPPCPPCPAPEFLGGP | chain |
| | SVFLFPPKPKDTLMISRTPEVTCVVVDVSQEDPEVQFNWYVDGVEVHN | WT IL2 with R38W, fuse |
| | AKTKPREEQFNSTYRVVSVLTVLHQDWLNGKEYKCKVSNKGLPSSIEKTI | on N-terminal with no |
| | SKAKGQPREPQVYTLPPCQEEMTKNQVSLWCLVKGFYPSDIAVEWESN | linker |
| | GQPENNYKTTPPVLDSDGSFFLYSRLTVDKSRWQEGNVFSCSVMHEAL | |
| | HNHYTQKSLSLSLG | |
| 89 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 24.uIgG4V1, 1st heavy |
| | FMCEYADETATIVEFLNRWITFCQSIISKYGPPCPPCPAPEFLGGPSVFLFP | chain |
| | PKPKDTLMISRTPEVTCVVVDVSQEDPEVQFNWYVDGVEVHNAKTKP | WT IL2 with C-terminal |
| | REEQFNSTYRVVSVLTVLHQDWLNGKEYKCKVSNKGLPSSIEKTISKAKG | four amino acids deletion, |
| | QPREPQVYTLPPCQEEMTKNQVSLWCLVKGFYPSDIAVEWESNGQPE | fuse on N-terminal with no |
| | NNYKTTPPVLDSDGSFFLYSRLTVDKSRWQEGNVFSCSVMHEALHNHY | linker; and one amino acid |
| | TQKSLSLSLG | deletion in hIgG4 N- |
| | | terminal hinge |
| 90 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 25.uIgG4V1, 1st heavy |
| | FMCEYADETATIVEFLNRWITFCQSIIKYGPPCPPCPAPEFLGGPSVFLFPP | chain |
| | KPKDTLMISRTPEVTCVVVDVSQEDPEVQFNWYVDGVEVHNAKTKPR | WT IL2 with C-terminal |
| | EEQFNSTYRVVSVLTVLHQDWLNGKEYKCKVSNKGLPSSIEKTISKAKGQ | four amino acids deletion, |
| | PREPQVYTLPPCQEEMTKNQVSLWCLVKGFYPSDIAVEWESNGQPENN | fuse on N-terminal with no |
| | YKTTPPVLDSDGSFFLYSRLTVDKSRWQEGNVFSCSVMHEALHNHYTQ | linker; and two amino acid |
| | KSLSLSLG | deletion in hIgG4 N- |
| | | terminal hinge |
| 91 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 26.uIgG4V1, 1st heavy |
| | FMCEYADETATIVEFLNRWITFCQSILESKYGPPCPPCPAPEFLGGPSVFLF | chain |
| | PPKPKDTLMISRTPEVTCVVVDVSQEDPEVQFNWYVDGVEVHNAKTK | WT IL2 with C-terminal |
| | PREEQFNSTYRVVSVLTVLHQDWLNGKEYKCKVSNKGLPSSIEKTISKAK | four amino acids deletion |
| | GQPREPQVYTLPPCQEEMTKNQVSLWCLVKGFYPSDIAVEWESNGQP | and I129L, fuse on N- |
| | ENNYKTTPPVLDSDGSFFLYSRLTVDKSRWQEGNVFSCSVMHEALHNH | terminal with no linker |
| | YTQKSLSLSLG | |
| 92 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 27.uIgG4V1, 1st heavy |
| | FMCEYADETATIVEFLNRWITFCQSIVESKYGPPCPPCPAPEFLGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion |
| | KGQPREPQVYTLPPCQEEMTKNQVSLWCLVKGFYPSDIAVEWESNGQP | and I129V, fuse on N- |
| | ENNYKTTPPVLDSDGSFFLYSRLTVDKSRWQEGNVFSCSVMHEALHNH | terminal with no linker |
| | YTQKSLSLSLG | |
| 93 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 28.uIgG4V1, 1st heavy |
| | FMCEYADETATIVEFLNRWITFCQSIAESKYGPPCPPCPAPEFLGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion |
| | KGQPREPQVYTLPPCQEEMTKNQVSLWCLVKGFYPSDIAVEWESNGQP | and I129A, fuse on N- |
| | ENNYKTTPPVLDSDGSFFLYSRLTVDKSRWQEGNVFSCSVMHEALHNH | terminal with no linker |
| | YTQKSLSLSLG | |
| 94 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 29.uIgG4V1, 1st heavy |
| | FMCEYADETATIVEFLNRWITFCQSISESKYGPPCPPCPAPEFLGGPSVFLF | chain |
| | PPKPKDTLMISRTPEVTCVVVDVSQEDPEVQFNWYVDGVEVHNAKTK | WT IL2 with C-terminal |
| | PREEQFNSTYRVVSVLTVLHQDWLNGKEYKCKVSNKGLPSSIEKTISKAK | four amino acids deletion |
| | GQPREPQVYTLPPCQEEMTKNQVSLWCLVKGFYPSDIAVEWESNGQP | and I129S, fuse on N- |
| | ENNYKTTPPVLDSDGSFFLYSRLTVDKSRWQEGNVFSCSVMHEALHNH | terminal with no linker |
| | YTQKSLSLSLG | |
| 95 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 30.uIgG4V1, 1st heavy |
| | FMCEYADETATIVEFLNRWITFCQSITESKYGPPCPPCPAPEFLGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion |
| | KGQPREPQVYTLPPCQEEMTKNQVSLWCLVKGFYPSDIAVEWESNGQP | and I129T, fuse on N- |
| | ENNYKTTPPVLDSDGSFFLYSRLTVDKSRWQEGNVFSCSVMHEALHNH | terminal with no linker |
| | YTQKSLSLSLG | |
| 96 | APTSSSTKKTQLQLEHLLLDLQMILNGINNYKNPKLTRMLTVKFYMPKKA | W3XX115-T2U0.E44- |
| | TELKHLQCLEEELKPLEEVLNLAQSKNFHLRPRDLISNINVIVLELKGSETT | 31.uIgG4V322, 1st heavy |
| | FMCEYADETATIVEFLNRWITFCQSILESKYGPPCPPCPAPEAAGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids |
| | KGQPREPQVYTLPPCQEEMTKNQVSLWCLVKGFYPSDIAVEWESNGQP | deletion ,I129T, F42V, fuse |
| | ENNYKTTPPVLDSDGSFFLYSRLTVDKSRWQEGNVFSCSVMHEALHNH | on N-terminal with no |
| | YTQKSLSLSLG | linker |
| 97 | APTSSSTKKTQLQLEHLLLDLQMILNGINNYKNPKLTRMLTIKFYMPKKA | W3XX115-T2U0.E44- |
| | TELKHLQCLEEELKPLEEVLNLAQSKNFHLRPRDLISNINVIVLELKGSETT | 32.uIgG4V322, 1st heavy |
| | FMCEYADETATIVEFLNRWITFCQSILESKYGPPCPPCPAPEAAGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids |
| | KGQPREPQVYTLPPCQEEMTKNQVSLWCLVKGFYPSDIAVEWESNGQP | deletion, I129T, F42I, fuse |
| | ENNYKTTPPVLDSDGSFFLYSRLTVDKSRWQEGNVFSCSVMHEALHNH | on N-terminal with no |
| | YTQKSLSLSLG | linker |
| 98 | APTSSSTKKTQLQLEHLLLDLQMILNGINNYKNPKLTWMLTFKFYMPKK | W3XX115-T2U0.E44- |
| | ATELKHLQCLEEELKPLEEVLNLAQSKNFHLRPRDLISNINVIVLELKGSET | 33.uIgG4V322, 1st heavy |
| | TFMCEYADETATIVEFLNRWITFCQSILESKYGPPCPPCPAPEAAGGPSVF | chain |
| | LFPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | I129T, R38W, fuse on N- |
| | ENNYKTTPPVLDSDGSFFLYSRLTVDKSRWQEGNVFSCSVMHEALHNH | terminal with no linker |
| | YTQKSLSLSLG | |
| 99 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 34.uIgG4V322, 1st heavy |
| | FMCEYADETATIVEFLNRWITFCQAIIESKYGPPCPPCPAPEAAGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | S127A, fuse on N-terminal |
| | ENNYKTTPPVLDSDGSFFLYSRLTVDKSRWQEGNVFSCSVMHEALHNH | with no linker |
| | YTQKSLSLSLG | |
| 100 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 35.uIgG4V322, 1st heavy |
| | FMCEYADETATIVEFLNRWITFCQTIIESKYGPPCPPCPAPEAAGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | S127T, fuse on N-terminal |
| | ENNYKTTPPVLDSDGSFFLYSRLTVDKSRWQEGNVFSCSVMHEALHNH | with no linker |
| | YTQKSLSLSLG | |
| 101 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 36.uIgG4V322, 1st heavy |
| | FMCEYADETATIVEFLNRWITFCQDIIESKYGPPCPPCPAPEAAGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | S127D, fuse on N-terminal |
| | ENNYKTTPPVLDSDGSFFLYSRLTVDKSRWQEGNVFSCSVMHEALHNH | with no linker |
| | YTQKSLSLSLG | |
| 102 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 37.uIgG4V322, 1st heavy |
| | FMCEYADETATIVEFLNRWITFCQNISESKYGPPCPPCPAPEAAGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | S127N, I129S fuse on N- |
| | ENNYKTTPPVLDSDGSFFLYSRLTVDKSRWQEGNVFSCSVMHEALHNH | terminal with no linker |
| | YTQKSLSLSLG | |
| 103 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 38.uIgG4V322, 1st heavy |
| | FMCEYADETATIVEFLNRWITFAQSIIESKYGPPCPPCPAPEAAGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | C125A, fuse on N-terminal |
| | ENNYKTTPPVLDSDGSFFLYSRLTVDKSRWQEGNVFSCSVMHEALHNH | with no linker |
| | YTQKSLSLSLG | |
| 104 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 39.uIgG4V322, 1st heavy |
| | FMCEYADETATIVEFLNRWITFCQSAIESKYGPPCPPCPAPEAAGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | I128A, fuse on N-terminal |
| | ENNYKTTPPVLDSDGSFFLYSRLTVDKSRWQEGNVFSCSVMHEALHNH | with no linker |
| | YTQKSLSLSLG | |
| 105 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 40.uIgG4V322, 1st heavy |
| | FMCEYADRTATIVEFLNRWITFCQSIIESKYGPPCPPCPAPEAAGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | E110R, fuse on N-terminal |
| | ENNYKTTPPVLDSDGSFFLYSRLTVDKSRWQEGNVFSCSVMHEALHNH | with no linker |
| | YTQKSLSLSLG | |
| 106 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 41.uIgG4V322, 1st heavy |
| | FMCEYADITATIVEFLNRWITFCQSIIESKYGPPCPPCPAPEAAGGPSVFLF | chain |
| | PPKPKDTLMISRTPEVTCVVVDVSQEDPEVQFNWYVDGVEVHNAKTK | WT IL2 with C-terminal |
| | PREEQFNSTYRVVSVLTVLHQDWLNGKEYKCKVSNKGLPSSIEKTISKAK | four amino acids deletion, |
| | GQPREPQVYTLPPCQEEMTKNQVSLWCLVKGFYPSDIAVEWESNGQP | E110I, fuse on N-terminal |
| | ENNYKTTPPVLDSDGSFFLYSRLTVDKSRWQEGNVFSCSVMHEALHNH | with no linker |
| | YTQKSLSLSLG | |
| 107 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 42.uIgG4V322, 1st heavy |
| | FMCEYADTTATIVEFLNRWITFCQSIIESKYGPPCPPCPAPEAAGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | E110T, fuse on N-terminal |
| | ENNYKTTPPVLDSDGSFFLYSRLTVDKSRWQEGNVFSCSVMHEALHNH | with no linker |
| | YTQKSLSLSLG | |
| 108 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 43.uIgG4V322, 1st heavy |
| | FMCEYADETATIVEFLNRWYTFCQSIIESKYGPPCPPCPAPEAAGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | I122Y, fuse on N-terminal |
| | ENNYKTTPPVLDSDGSFFLYSRLTVDKSRWQEGNVFSCSVMHEALHNH | with no linker |
| | YTQKSLSLSLG | |
| 109 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 44.uIgG4V322, 1st heavy |
| | FMCEYADETATIVEFLNRWTTFCQSIIESKYGPPCPPCPAPEAAGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | I122T, fuse on N-terminal |
| | ENNYKTTPPVLDSDGSFFLYSRLTVDKSRWQEGNVFSCSVMHEALHNH | with no linker |
| | YTQKSLSLSLG | |
| 110 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 45.uIgG4V322, 1st heavy |
| | FMCEYADETATIVEFLNRWVTFCQSIIESKYGPPCPPCPAPEAAGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | I122V, fuse on N-terminal |
| | ENNYKTTPPVLDSDGSFFLYSRLTVDKSRWQEGNVFSCSVMHEALHNH | with no linker |
| | YTQKSLSLSLG | |
| 111 | APTSSSTKKTQLQLEHLLLDLQMILNGPNNYKNPKLTRMLTFKFYMPKK | W3XX115-T2U0.E44- |
| | ATELKHLQCLEEELKPLEEVLNLAQSKNFHLRPRDLISNINVIVLELKGSET | 46.uIgG4V322, 1st heavy |
| | TFMCEYADETATIVEFLNRWITFCQSIIESKYGPPCPPCPAPEAAGGPSVF | chain |
| | LFPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | I28P, fuse on N-terminal |
| | ENNYKTTPPVLDSDGSFFLYSRLTVDKSRWQEGNVFSCSVMHEALHNH | with no linker |
| | YTQKSLSLSLG | |
| 112 | APTSSSTKKTQLQLEHLLLDLQMILNGINNYDNPKLTRMLTFKFYMPKKA | W3XX115-T2U0.E44- |
| | TELKHLQCLEEELKPLEEVLNLAQSKNFHLRPRDLISNINVIVLELKGSETT | 47.uIgG4V322, 1st heavy |
| | FMCEYADETATIVEFLNRWITFCQSIIESKYGPPCPPCPAPEAAGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | K32D, fuse on N-terminal |
| | ENNYKTTPPVLDSDGSFFLYSRLTVDKSRWQEGNVFSCSVMHEALHNH | with no linker |
| | YTQKSLSLSLG | |
| 113 | APTSSSTKKTQLQLEHLLLDLQMILNGINNYKNPKLTRMLTFKFYMPKKA | W3XX115-T2U0.E44- |
| | TGLKHLQCLEEELKPLEEVLNLAQSKNFHLRPRDLISNINVIVLELKGSETT | 48.uIgG4V322, 1st heavy |
| | FMCEYADETATIVEFLNRWITFCQSIIESKYGPPCPPCPAPEAAGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | E52G, fuse on N-terminal |
| | ENNYKTTPPVLDSDGSFFLYSRLTVDKSRWQEGNVFSCSVMHEALHNH | with no linker |
| | YTQKSLSLSLG | |
| 114 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSRNFHLRPRDLISNINVIVLELKGSETT | 49.uIgG4V322, 1st heavy |
| | FMCEYADETATIVEFLNRWITFCQSIIESKYGPPCPPCPAPEAAGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | K76R, fuse on N-terminal |
| | ENNYKTTPPVLDSDGSFFLYSRLTVDKSRWQEGNVFSCSVMHEALHNH | with no linker |
| | YTQKSLSLSLG | |
| 115 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNGHLRPRDLISNINVIVLELKGSETT | 50.uIgG4V322, 1st heavy |
| | FMCEYADETATIVEFLNRWITFCQSIIESKYGPPCPPCPAPEAAGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | F78G, fuse on N-terminal |
| | ENNYKTTPPVLDSDGSFFLYSRLTVDKSRWQEGNVFSCSVMHEALHNH | with no linker |
| | YTQKSLSLSLG | |
| 116 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRYRDLISNINVIVLELKGSETT | 51.uIgG4V322, 1st heavy |
| | FMCEYADETATIVEFLNRWITFCQSIIESKYGPPCPPCPAPEAAGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | P82Y, fuse on N-terminal |
| | ENNYKTTPPVLDSDGSFFLYSRLTVDKSRWQEGNVFSCSVMHEALHNH | with no linker |
| | YTQKSLSLSLG | |
| 117 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 52.uIgG4V322, 1st heavy |
| | FMCEYADRTATIVEFLNRWITFCQSILESKYGPPCPPCPAPEAAGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | E110R and I129L, fuse on |
| | ENNYKTTPPVLDSDGSFFLYSRLTVDKSRWQEGNVFSCSVMHEALHNH | N-terminal with no linker |
| | YTQKSLSLSLG | |
| 118 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 53.uIgG4V322, 1st heavy |
| | FMCEYADITATIVEFLNRWITFCQSILESKYGPPCPPCPAPEAAGGPSVFLF | chain |
| | PPKPKDTLMISRTPEVTCVVVDVSQEDPEVQFNWYVDGVEVHNAKTK | WT IL2 with C-terminal |
| | PREEQFNSTYRVVSVLTVLHQDWLNGKEYKCKVSNKGLPSSIEKTISKAK | four amino acids deletion, |
| | GQPREPQVYTLPPCQEEMTKNQVSLWCLVKGFYPSDIAVEWESNGQP | E110I and I129L, fuse on |
| | ENNYKTTPPVLDSDGSFFLYSRLTVDKSRWQEGNVFSCSVMHEALHNH | N-terminal with no linker |
| | YTQKSLSLSLG | |
| 119 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 54.uIgG4V322, 1st heavy |
| | FMCEYADTTATIVEFLNRWITFCQSILESKYGPPCPPCPAPEAAGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | E110T and I129L, fuse on |
| | ENNYKTTPPVLDSDGSFFLYSRLTVDKSRWQEGNVFSCSVMHEALHNH | N-terminal with no linker |
| | YTQKSLSLSLG | |
| 120 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 55.uIgG4V322, 1st heavy |
| | FMCEYADETATIVEFLNRWYTFCQSILESKYGPPCPPCPAPEAAGGPSVF | chain |
| | LFPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | I122Y and I129L, fuse on |
| | ENNYKTTPPVLDSDGSFFLYSRLTVDKSRWQEGNVFSCSVMHEALHNH | N-terminal with no linker |
| | YTQKSLSLSLG | |
| 121 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 56.uIgG4V322, 1st heavy |
| | FMCEYADETATIVEFLNRWTTFCQSILESKYGPPCPPCPAPEAAGGPSVF | chain |
| | LFPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | I122T and I129L, fuse on |
| | ENNYKTTPPVLDSDGSFFLYSRLTVDKSRWQEGNVFSCSVMHEALHNH | N-terminal with no linker |
| | YTQKSLSLSLG | |
| 122 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 57.uIgG4V322, 1st heavy |
| | FMCEYADETATIVEFLNRWVTFCQSILESKYGPPCPPCPAPEAAGGPSVF | chain |
| | LFPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | I122V and I129L, fuse on |
| | ENNYKTTPPVLDSDGSFFLYSRLTVDKSRWQEGNVFSCSVMHEALHNH | N-terminal with no linker |
| | YTQKSLSLSLG | |
| 123 | APTSSSTKKTQLQLEHLLLDLQMILNGPNNYKNPKLTRMLTFKFYMPKK | W3XX115-T2U0.E44- |
| | ATELKHLQCLEEELKPLEEVLNLAQSKNFHLRPRDLISNINVIVLELKGSET | 58.uIgG4V322, 1st heavy |
| | TFMCEYADETATIVEFLNRWITFCQSILESKYGPPCPPCPAPEAAGGPSVF | chain |
| | LFPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | I28P and I129L, fuse on N- |
| | ENNYKTTPPVLDSDGSFFLYSRLTVDKSRWQEGNVFSCSVMHEALHNH | terminal with no linker |
| | YTQKSLSLSLG | |
| 124 | APTSSSTKKTQLQLEHLLLDLQMILNGINNYDNPKLTRMLTFKFYMPKKA | W3XX115-T2U0.E44- |
| | TELKHLQCLEEELKPLEEVLNLAQSKNFHLRPRDLISNINVIVLELKGSETT | 59.uIgG4V322, 1st heavy |
| | FMCEYADETATIVEFLNRWITFCQSILESKYGPPCPPCPAPEAAGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | K32D and I129L, fuse on |
| | ENNYKTTPPVLDSDGSFFLYSRLTVDKSRWQEGNVFSCSVMHEALHNH | N-terminal with no linker |
| | YTQKSLSLSLG | |
| 125 | APTSSSTKKTQLQLEHLLLDLQMILNGINNYKNPKLTRMLTFKFYMPKKA | W3XX115-T2U0.E44- |
| | TGLKHLQCLEEELKPLEEVLNLAQSKNFHLRPRDLISNINVIVLELKGSETT | 60.uIgG4V322, 1st heavy |
| | FMCEYADETATIVEFLNRWITFCQSILESKYGPPCPPCPAPEAAGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | E52G and I129L, fuse on |
| | ENNYKTTPPVLDSDGSFFLYSRLTVDKSRWQEGNVFSCSVMHEALHNH | N-terminal with no linker |
| | YTQKSLSLSLG | |
| 126 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSRNFHLRPRDLISNINVIVLELKGSETT | 61.uIgG4V322, 1st heavy |
| | FMCEYADETATIVEFLNRWITFCQSILESKYGPPCPPCPAPEAAGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | K76R and I129L, fuse on |
| | ENNYKTTPPVLDSDGSFFLYSRLTVDKSRWQEGNVFSCSVMHEALHNH | N-terminal with no linker |
| | YTQKSLSLSLG | |
| 127 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNGHLRPRDLISNINVIVLELKGSETT | 62.uIgG4V322, 1st heavy |
| | FMCEYADETATIVEFLNRWITFCQSILESKYGPPCPPCPAPEAAGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | F78G and I129L, fuse on |
| | ENNYKTTPPVLDSDGSFFLYSRLTVDKSRWQEGNVFSCSVMHEALHNH | N-terminal with no linker |
| | YTQKSLSLSLG | |
| 128 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRYRDLISNINVIVLELKGSETT | 63.uIgG4V322, 1st heavy |
| | FMCEYADETATIVEFLNRWITFCQSILESKYGPPCPPCPAPEAAGGPSVFL | chain |
| | FPPKPKDTLMISRTPEVTCVVVDVSQEDPEVQFNWYVDGVEVHNAKT | WT IL2 with C-terminal |
| | KPREEQFNSTYRVVSVLTVLHQDWLNGKEYKCKVSNKGLPSSIEKTISKA | four amino acids deletion, |
| | KGQPREPQVYTLPPCQEEMTKNQVSLWCLVKGFYPSDIAVEWESNGQP | P82Y and I129L, fuse on |
| | ENNYKTTPPVLDSDGSFFLYSRLTVDKSRWQEGNVFSCSVMHEALHNH | N-terminal with no linker |
| | YTQKSLSLSLG | |
| 129 | APTSSSTKKTQLQLEHLLLDLQMILNGINNYKNPKLTRMLTFKFYMPKKA | W3XX115-T2U0.E44- |
| | TELKHLQCLEEELKPLEEVLNLAQSKNFHLRPRDLISNINVIVLELKGSETT | 1.uIgG4V322, 1st heavy |
| | FMCEYADETATIVEFLNRWITFCQSIISTLTESKYGPPCPPCPAPEAAGGPS | chain |
| | VFLFPPKPKDTLMISRTPEVTCVVVDVSQEDPEVQFNWYVDGVEVHNA | WT IL2 fuse on N- |
| | KTKPREEQFNSTYRVVSVLTVLHQDWLNGKEYKCKVSNKGLPSSIEKTIS | terminal with no linker, |
| | KAKGQPREPQVYTLPPCQEEMTKNQVSLWCLVKGFYPSDIAVEWESNG | FALA mutation on hIgG4 |
| | QPENNYKTTPPVLDSDGSFFLYSRLTVDKSRWQEGNVFSCSVMHEALH | Fc |
| | NHYTQKSLSLSLG | |
| 130 | APTSSSTKKTQLQLEHLLLDLQMILNGINNYKNPKLTRMLTAKFAMPKKA | W3XX115-T3U0.E44- |
| | TELKHLQCLEEELKPLEEVLNGAQSKNFHLRPRDLISNINVIVLELKGSETT | 1.uIgG4V322, 1st heavy |
| | FMCEYADETATIVEFLNRWITFCQSIISTLTESKYGPPCPPCPAPEAAGGPS | chain |
| | VFLFPPKPKDTLMISRTPEVTCVVVDVSQEDPEVQFNWYVDGVEVHNA | IL2 variant fuse on N- |
| | KTKPREEQFNSTYRVVSVLTVLHQDWLNGKEYKCKVSNKGLPSSIEKTIS | terminal with no linker, |
| | KAKGQPREPQVYTLPPCQEEMTKNQVSLWCLVKGFYPSDIAVEWESNG | FALA mutation on hIgG4 |
| | QPENNYKTTPPVLDSDGSFFLYSRLTVDKSRWQEGNVFSCSVMHEALH | Fc |
| | NHYTQKSLSLSLG | |
| 131 | EIQLQQSGPELVKPGASVKVSCKASGYSFTDYNMYWVKQSHGESLEWI | E44 uIgG1V320 format, |
| | GYIDPYNGGTRYNQKFKGKATLTVDKSSSTAFMHLNSLTSEDSAVYYCAR | 2^{nd} heavy chain |
| | NGHWDGAWFAYWGQGTLVTVSAASTKGPSVFPLAPSSKSTSGGTAAL | |
| | GCLVKDYFPEPVTVSWNSGALTSGVHTFPAVLQSSGLYSLSSVVTVPSSSL | |
| | GTQTYICNVNHKPSNTKVDKRVEPKSCDKTHTCPPCPAPEAAGGPSVFL | |
| | FPPKPKDTLMISRTPEVTCVVVDVSHEDPEVKFNWYVDGVEVHNAKTK | |
| | PREEQYNSTYRVVSVLTVLHQDWLNGKEYKCKVSNKALPAPIEKTISKAK | |
| | GQPREPQVCTLPPSREEMTKNQVSLSCAVKGFYPSDIAVEWESNGQPE | |
| | NNYKTTPPVLDSDGSFFLVSKLTVDKSRWQQGNVFSCSVMHEALHNHY | |
| | TQKSLSLSPG | |
| 132 | EIQLQQSGPELVKPGASVKVSCKASGYSFTDYNMYWVKQSHGESLEWI | E44 uIgG4V1 format, 2^{nd} |
| | GYIDPYNGGTRYNQKFKGKATLTVDKSSSTAFMHLNSLTSEDSAVYYCAR | heavy chain |
| | NGHWDGAWFAYWGQGTLVTVSAASTKGPSVFPLAPCSRSTSESTAALG | |
| | CLVKDYFPEPVTVSWNSGALTSGVHTFPAVLQSSGLYSLSSVVTVPSSSLG | |
| | TKTYTCNVDHKPSNTKVDKRVESKYGPPCPPCPAPEFLGGPSVFLFPPKP | |
| | KDTLMISRTPEVTCVVVDVSQEDPEVQFNWYVDGVEVHNAKTKPREE | |
| | QFNSTYRVVSVLTVLHQDWLNGKEYKCKVSNKGLPSSIEKTISKAKGQPR | |
| | EPQVCTLPPSQEEMTKNQVSLSCAVKGFYPSDIAVEWESNGQPENNYK | |
| | TTPPVLDSDGSFFLVSRLTVDKSRWQEGNVFSCSVMHEALHNHYTQKSL | |
| | SLSLG | |
| 133 | EIQLQQSGPELVKPGASVKVSCKASGYSFTDYNMYWVKQSHGESLEWI | E44 uIgG4V322 format, |
| | GYIDPYNGGTRYNQKFKGKATLTVDKSSSTAFMHLNSLTSEDSAVYYCAR | 2^{nd} heavy chain |
| | NGHWDGAWFAYWGQGTLVTVSAASTKGPSVFPLAPCSRSTSESTAALG | |
| | CLVKDYFPEPVTVSWNSGALTSGVHTFPAVLQSSGLYSLSSVVTVPSSSLG | |
| | TKTYTCNVDHKPSNTKVDKRVESKYGPPCPPCPAPEAAGGPSVFLFPPK | |
| | PKDTLMISRTPEVTCVVVDVSQEDPEVQFNWYVDGVEVHNAKTKPRE | |
| | EQFNSTYRVVSVLTVLHQDWLNGKEYKCKVSNKGLPSSIEKTISKAKGQP | |
| | REPQVCTLPPSQEEMTKNQVSLSCAVKGFYPSDIAVEWESNGQPENNY | |
| | KTTPPVLDSDGSFFLVSRLTVDKSRWQEGNVFSCSVMHEALHNHYTQK | |
| | SLSLSLG | |
| 134 | DIQMNQSPSSLSASLGDTITITCHASQNINVWLSWYQQKPGNIPKLLIYK | Same light chain for E44, |
| | ASNLHTGVPSRFSGSRSGTGFTLTISSLQPEDIATYYCHQGQSYPFTFGSG | F114 and Z80 formats |
| | TKLEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGL | |
| | SSPVTKSFNRGEC | |
| 135 | ELCDDDPPEIPHATFKAMAYKEGTMLNCECKRGFRRIKSGSLYMLCTGN | W3XX115- |
| | SSHSSWDNQCQCTSSATRNTTKQVTPQPEEQKERKTTEMQSPMQPV | hPro1.ECD.His, IL2Rα |
| | DQASLPGHCREPPPWENEATERIYHFVVGQMVYYQCVQGYRALHRGP | ECD |
| | AESVCKMTHGKTRWTQPQLICTGEMETSQFPGEEKPQASPEGRPESET | |
| | SCHHHHHH | |
| 136 | AVNGTSQFTCFYNSRANISCVWSQDGALQDTSCQVHAWPDRRRWNQ | W369-hPro2.ECD.His, |
| | TCELLPVSQASWACNLILGAPDSQKLTTVDIVTLRVLCREGVRWRVMAI | IL2Rβ ECD |
| | QDFKPFENLRLMAPISLQVVHVETHRCNISWEISQASHYFERHLEFEART | |
| | LSPGHTWEEAPLLTLKQKQEWICLETLTPDTQYEFQVRVKPLQGEFTTW | |
| | SPWSQPLAFRTKPAALGKDLEGGGGSGGGGHHHHHH | |
| 137 | LNTTILTPNGNEDTTADFFLTTMPTDSLSVSTLPLPEVQCFVFNVEYMNC | W369-hPro1.ECD.His, IL- |
| | TWNSSSEPQPTNLTLHYWYKNSDNDKVQKCSHYLFSEEITSGCQLQKKE | 2R γ ECD |
| | IHLYQTFVVQLQDPREPRRQATQMLKLQNLVIPWAPENLTLHKLSESQLE | |
| | LNWNNRFLNHCLEHLVQYRTDWDHSWTEQSVDYRHKFSLPSVDGQK | |
| | RYTFRVRSRFNPLCGSAQHWSEWSHPIHWGSNTSKENLEGGGGSGGG | |
| | GHHHHHH | |
| 138 | APTSSSTKKTQLQLEHLLLDLQMILNGINNYKNPKLTRMLTFKFYMPKKA | W3XX115-T2.Z20- |
| | TELKHLQCLEEELKPLEEVLNLAQSKNFHLRPRDLISNINVIVLELKGSETT | 1.uIgG4V322, heavy chain |
| | FMCEYADETATIVEFLNRWITFCQSIIESKYGPPCPPCPAPEAAGGPSVFL | WT IL2 with C-terminal |
| | FPPKPKDTLMISRTPEVTCVVVDVSQEDPEVQFNWYVDGVEVHNAKT | four amino acids deletion, |
| | KPREEQFNSTYRVVSVLTVLHQDWLNGKEYKCKVSNKGLPSSIEKTISKA | fuse on N-terminal with no |
| | KGQPREPQVYTLPPSQEEMTKNQVSLTCLVKGFYPSDIAVEWESNGQPE | linker |
| | NNYKTTPPVLDSDGSFFLYSRLTVDKSRWQEGNVFSCSVMHEALHNHY | |
| | TQKSLSLSLGK | |
| 139 | APTSSSTKKTQLQLEHLLLDLQMILNGINNYKNPKLTRMLTFKFYMPKKA | W3XX115-T2.Z20- |
| | TELKHLQCLEEELKPLEEVLNLAQSKNFHLRPRDLISNINVIVLELKGSETT | 2.uIgG4V322, heavy chain |
| | FMCEYADETATIVEFLNRWITFCQSILESKYGPPCPPCPAPEAAGGPSVFL | WT IL2 with C-terminal |
| | FPPKPKDTLMISRTPEVTCVVVDVSQEDPEVQFNWYVDGVEVHNAKT | four amino acids deletion |
| | KPREEQFNSTYRVVSVLTVLHQDWLNGKEYKCKVSNKGLPSSIEKTISKA | and I129L, fuse on N- |
| | KGQPREPQVYTLPPSQEEMTKNQVSLTCLVKGFYPSDIAVEWESNGQPE | terminal with no linker |
| | NNYKTTPPVLDSDGSFFLYSRLTVDKSRWQEGNVFSCSVMHEALHNHY | |
| | TQKSLSLSLGK | |
| 140 | APASSSTKKTQLQLEHLLLNLQMILNGINNYKNPKLTRMLTFKFYMPKKA | W3XX115-BMK1, E44 |
| | TELKHLQCLEEELKPLEEVLKLAQSKNFHLRPRDLISNINVIVLELKGSETTF | format, 1st heavy chain |
| | MCEYADETATIVEFLNRWITFSQSIISTLTESKYGPPCPPCPAPEFLGGPSV | WT IL2 with T3A, D20N, |
| | FLFPPKPKDTLMISRTPEVTCVVVDVSQEDPEVQFNWYVDGVEVHNAK | N71K, C125S fuse on N- |
| | TKPREEQFNSTYRVVSVLTVLHQDWLNGKEYKCKVSNKGLPSSIEKTISK | terminal with no linker, |
| | AKGQPREPQVYTLPPCQEEMTKNQVSLWCLVKGFYPSDIAVEWESNG | FALA mutation on hIgG4 |
| | QPENNYKTTPPVLDSDGSFFLYSRLTVDKSRWQEGNVFSCSVMHEALH | Fc |
| | NHYTQKSLSLSLG | |
| 141 | APTSSSTSSSTAEAQQQQQHLEQLRMDLEELLSRMENYRNLKLPRMLTF | W3XX115-BMK7, E44 |
| | KFYLPKQATELKDLQCLEDELGPLRHVLDLTQSKSFQLEDAENFISNIRVT | format, 1st heavy chain, |
| | VVKLKGSDNTFECQFDDESATVVDFLRRWIAFCHSIISTSPQESKYGPPCP | mouse IL2 with 41-46, 91, |
| | PCPAPEFLGGPSVFLFPPKPKDTLMISRTPEVTCVVVDVSQEDPEVQFN | 101, 141 & 169 amino acids |
| | WYVDGVEVHNAKTKPREEQFNSTYRVVSVLTVLHQDWLNGKEYKCKV | deletion and L53R, |
| | SNKGLPSSIEKTISKAKGQPREPQVYTLPPCQEEMTKNQVSLWCLVKGFY | Q162H, fuse on N-terminal |
| | PSDIAVEWESNGQPENNYKTTPPVLDSDGSFFLYSRLTVDKSRWQEGN | with no linker, FALA |
| | VFSCSVMHEALHNHYTQKSLSLSLG | mutation on hIgG4 Fc |
| 142 | APTSSSTKKTQLQLEHLRLDLEMILNGINNYKNPKLTRMLTFKFYMPKKA | W3XX115-BMK8, E44 |
| | TELKHLQCLEEELKPLEEVLNLAQSKNFHLRPRDLISNINVIVLELKGSETT | format, 1st heavy chain |
| | FMCEYADETATIVEFLNRWITFCKSIISTLTESKYGPPCPPCPAPEFLGGPS | WT IL2 with L18R, Q22E, |
| | VFLFPPKPKDTLMISRTPEVTCVVVDVSQEDPEVQFNWYVDGVEVHNA | Q126K fuse on N-terminal |
| | KTKPREEQFNSTYRVVSVLTVLHQDWLNGKEYKCKVSNKGLPSSIEKTIS | with no linker, FALA |
| | KAKGQPREPQVYTLPPCQEEMTKNQVSLWCLVKGFYPSDIAVEWESNG | mutation on hIgG4 Fc |
| | QPENNYKTTPPVLDSDGSFFLYSRLTVDKSRWQEGNVFSCSVMHEALH | |
| | NHYTQKSLSLSLG | |
| 143 | APTSSSTKKTQLQLEHLLHDLQMILNGINNYKNPKLTRMLTFKFYMPKK | W3XX115-BMK9, E44 |
| | ATELKHLQCLEEELKPLEEVLNLAQSKNFHLRPRDLISNINVIVLELKGSET | format, 1st heavy chain |
| | TFMCEYADETATIVEFLNRWITFIESIISTLTESKYGPPCPPCPAPEFLGGPS | WT IL2 with L19D, C125I, |
| | VFLFPPKPKDTLMISRTPEVTCVVVDVSQEDPEVQFNWYVDGVEVHNA | Q126E fuse on N-terminal |
| | KTKPREEQFNSTYRVVSVLTVLHQDWLNGKEYKCKVSNKGLPSSIEKTIS | with no linker, FALA |
| | KAKGQPREPQVYTLPPCQEEMTKNQVSLWCLVKGFYPSDIAVEWESNG | mutation on hIgG4 Fc |
| | QPENNYKTTPPVLDSDGSFFLYSRLTVDKSRWQEGNVFSCSVMHEALH | |
| | NHYTQKSLSLSLG | |
| 144 | APTSSSTKKTQLQLEHLLLDLQMILNGINNYKNPKLTRMLTFK | W3XX115-T2U0.Z80- |
| | FYMPKKATELKHLQCLEEELKPLEEVLNLAQSKNFHLRPRDLIS | 6.uIgG4V322, heavy chain |
| | NINVIVLELKGSETTFMCEYADETATIVEFLNRWITFCQSIIESK | WT IL2 with C-terminal |
| | YGPPCPPCPAPEAAGGPSVFLFPPKPKDTLMISRTPEVTCVVV | four amino acids deletion, |
| | DVSQEDPEVQFNWYVDGVEVHNAKTKPREEQFNSTYRVVS | fuse on N-terminal with no |
| | VLTVLHQDWLNGKEYKCKVSNKGLPSSIEKTISKAKGQPREP | linker, Fab fuse on C |
| | QVYTLPPSQEEMTKNQVSLTCLVKGFYPSDIAVEWESNGQPE | terminal with (G4S)4 |
| | NNYKTTPPVLDSDGSFFLYSRLTVDKSRWQEGNVFSCSVMH | linker, FALA mutation on |
| | EALHNHYTQKSLSLSLGKGGGGSGGGGSGGGGSGGGGSEIQ | hIgG4 Fc |
| | LQQSGPELVKPGASVKVSCKASGYSFTDYNMYWVKQSHGES | |
| | LEWIGYIDPYNGGTRYNQKFKGKATLTVDKSSSTAFMHLNSLT | |
| | SEDSAVYYCARNGHWDGAWFAYWGQGTLVTVSA | |
| 145 | APTSSSTKKTQLQLEHLLLDLQMILNGINNYKNPKLTRMLTFK | W3XX115-T2U0. Z80- |
| | FYMPKKATELKHLQCLEEELKPLEEVLNLAQSKNFHLRPRDLIS | 26.uIgG4V322, heavy |
| | NINVIVLELKGSETTFMCEYADETATIVEFLNRWITFCQSILESK | chain |
| | YGPPCPPCPAPEAAGGPSVFLFPPKPKDTLMISRTPEVTCVVV | WT IL2 with C-terminal |
| | DVSQEDPEVQFNWYVDGVEVHNAKTKPREEQFNSTYRVVS | four amino acids deletion |
| | VLTVLHQDWLNGKEYKCKVSNKGLPSSIEKTISKAKGQPREP | and I129L, fuse on N- |
| | QVYTLPPSQEEMTKNQVSLTCLVKGFYPSDIAVEWESNGQPE | terminal with no linker, |
| | NNYKTTPPVLDSDGSFFLYSRLTVDKSRWQEGNVFSCSVMH | Fab fuse on C terminal |
| | EALHNHYTQKSLSLSLGKGGGGSGGGGSGGGGSGGGGSEIQ | with (G4S)4 linker, FALA |
| | LQQSGPELVKPGASVKVSCKASGYSFTDYNMYWVKQSHGES | mutation on hIgG4 Fc |
| | LEWIGYIDPYNGGTRYNQKFKGKATLTVDKSSSTAFMHLNSLT | |
| | SEDSAVYYCARNGHWDGAWFAYWGQGTLVTVSA | |

### EXAMPLES

The present disclosure, thus generally described, will be understood more readily by reference to the following Examples, which are provided by way of illustration and are not intended to be limiting of the present disclosure. The Examples are not intended to represent that the experiments below are all or the only experiments performed.

### Example 1

### Preparation of Materials, IL-2 Variants and Fusion Proteins

### 1.1 Preparation of Materials

Information on the commercially available materials used in the Examples is provided in Table 1.

TABLE 1

| Materials | Vendor | Cat |
|---|---|---|
| ExpiFectamine ™ 293 | Invitrogen | A14525 |
| Transfection Kit | | |
| Opti-MEM | Invitrogen | 31985070 |
| Expi293 Expression Medium | Invitrogen | A1435101 |
| ExpiFectamine ™ 293 cell | Invitrogen | A14635 |
| Ni column | GE healthcare | 173712 |
| Protein A column | GE healthcare | 175438 |
| HPLC-SEC | TOSOH | 0008541 |
| NuPAGE4%-12% Bis-Tris Gel | Thermo Fisher | NP0322BOX |
| ExpiFectamine ™ 293 | Invitrogen | A14525 |
| Transfection Kit | | |
| Opti-MEM | Invitrogen | 31985070 |
| Expi293 Expression Medium | Invitrogen | A1435101 |
| HH cell | ATCC | CRL2105 |
| NK92 cell | ATCC | CRL2105 |
| PE-Anti Human CD4 | Invitrogen | 12-0049-42 |
| FITC-Anti Human CD3 | Bio legend | 317306.00 |
| APC-Cy7 Mouse Anti-Human CD8 | BD Biosciences | Pharmingen-557834 |
| Alexa Fluor 647 mouse anti- | BD Biosciences | 562076 |
| Stat5 (pY694) | | |
| APC-Anti Human Foxp3 | BioLegend | 17-4776-42 |
| PE-Anti Human CD25 clone BC96 | BioLegend | 302606 |
| BD Phosflow ™ Fix Buffer I | BD Biosciences | BDB557870 |
| Phosflow Perm Buffer III | BD Biosciences | BDB558050 |
| Treg expansion kit, human | Miltenyi | 130-095-345 |
| EasySep Human CD4+ CD127low | Stemcell | 18063 |
| CD25+ Regulatory T cell | | |
| isolation Kit | | |
| Recombinant Human Interleu- | Si Huan Sheng Wu | |
| kin-2 Injecition | | |
| Goat anti-human IgG Fc R-PE | Jackson | 109-115-098 |
| EasySep. Human CD8+ T Cell | Stemcell | 17953 |
| Isolation Kit | | |
| BV510 anti-mouse CD45, 30-F11 | BioLegend | 103138 |
| BV786 anti-mouse CD3, 500A1 | BD Biosciences | 740854 |
| BV421 anti-mouse CD4, RM4-4 | BD Biosciences | 740008 |
| BUV661 anti-mouse CD8, 53-6.7 | BD Biosciences | 740854 |
| PE-Cy7 anti-mouse CD25, PC61 | BioLegend | 102016 |
| BV650 anti-mouse CD44, IM7 | BD Biosciences | 740455 |
| BUV395 anti-mouse CD62L, MEL-14 | BD Biosciences | 740218 |
| PE-Cyanine5 anti-mouse NK1.1 | BioLegend | 103138 |
| AF700 anti-Ki67, 16A8 | BioLegend | 652420 |
| PE anti-mouse Foxp3, MF-14 | BioLegend | 126404 |
| FITC anti-mouse GranzymeB, GB11 | BioLegend | 515403 |
| APC anti-mouse Tbet, 4B10 | BioLegend | 644814 |

### 1.2 Generation of IL-2/Fc Fusion Proteins Comprising Wild Type IL-2 or IL-2 Variants

### Construction of Plasmids for Expression of IL-2/Fe Fusion Proteins

The structures of the IL-2/Fc fusion proteins are as shown in FIG. 1. The E44 format indicates the IL-2 moiety is linked at the N terminal of the Fc region with a linker (further referred to as “E44-2”) or without a linker (further referred to as “E44-1”). The F114-1 format indicates that the IL-2 moiety is linked at the C terminal of the Fc region with a linker. The Z20 format indicates that two IL-2 moieties are each linked at the N terminal of the Fc region without a linker.

TABLE 2
Different formats of Fc fusion IL-2 proteins

| Isoform | IL-2 site | linker |
|---|---|---|
| F114-1 | C terminal of Fc | (G4S)3 or (G4S)2 |
| E44-2 | N terminal of Fc | (G4S)3 or (G4S)2 |
| E44-1 | N terminal of Fc | no linker |
| Z20 | N terminal of Fc | no linker |

Polynucleotides encoding the VL, VH, Ck, CH1 and VHH of a selected antibody, were respectively amplified by PCR from DNA templates. Polynucleotides encoding wild type IL-2 and IL-2 variants were synthesized by Sangon Biotech Inc. Polynucleotides encoding native light chain sequences of the antibodies were inserted into a linearized vector containing a CMV promoter and a kappa or lambda signal peptide. The DNA fragments of anti-target VH-CH1 and wild-type IL-2 or IL-2 variants were inserted into a linearized vector, which contains the constant region CH2-CH3 of human IgG1 or IgG4 with or without a (G4S) n linker, or hinge truncated IgG4 according to the formats. The vector contains a CMV promoter and a human antibody heavy chain signal peptide.

The generated IL-2/Fc fusion proteins and their corresponding IL-2 format (WT or variant) are listed in Table 3. The ID of the IL-2/Fc fusion proteins indicate their structure: “E44” or “F114” specifies whether the IL-2 moiety is at the N terminal or C terminal of the Fc region, “Z20” refer to bivalent IL-2 moiety, “uIgG1V320”, “uIgG4V322” and “uIgG4V1” refer to IgG1 Fc with LALA mutation, IgG4 Fc with FALA mutation and wild-type IgG4 Fc, respectively. The prefix “W3xx115-” can be omitted for simplicity.

TABLE 3
IL-2/Fc fusion proteins

| | | Linkage | |
|---|---|---|---|
| | | between | Antigen- |
| | | IL-2 moiety | binding |
| Protein ID | IL-2 moiety | and Fc | moiety |
| W3xx115-U0T2.F114- | WT | (G4S)2/(G4S)3 | Fab |
| 1.uIgG1V320 | | linker | |
| W3xx115-U0T3.F114- | F42V/Y45A/L72G (Roche) | (G4S)2/(G4S)3 | Fab |
| 1.uIgG1V320 | | linker | |
| W3xx115-T2U0.E44- | WT | no linker | Fab |
| 1.uIgG4V1 | | | |
| W3xx115-T2U0.E44- | WT | (G4S)2/(G4S)3 | Fab |
| 2.uIgG1V320 | | linker | |
| W3xx115-T3U0.E44- | F42V/Y45A/L72G (Roche) | no linker | Fab |
| 1.uIgG4V1 | | | |
| W3xx115-T3U0.E44- | F42V/Y45A/L72G (Roche) | (G4S)2/(G4S)3 | Fab |
| 2.uIgG1V320 | | linker | |
| W3xx115-T2U0.E44- | C terminal truncation: | no linker | Fab |
| 3.uIgG4V1 | 1 amino acid | | |
| W3xx115-T2U0.E44- | C terminal truncation: | no linker | Fab |
| 4.uIgG4V1 | 2 amino acids | | |
| W3xx115-T2U0.E44- | C terminal truncation: | no linker | Fab |
| 5.uIgG4V1 | 3 amino acids | | |
| W3xx115-T2U0.E44- | C terminal truncation: | no linker | Fab |
| 6.uIgG4V1 | 4 amino acids | | |
| W3xx115-T2U0.E44- | C terminal truncation: | no linker | Fab |
| 7.uIgG4V1 | 5 amino acids | | |
| W3xx115-T2U0.E44- | C terminal truncation: | no linker | Fab |
| 8.uIgG4V1 | 6 amino acids | | |
| W3xx115-T2U0.E44- | C terminal truncation: | no linker | Fab |
| 9.uIgG4V1 | 7 amino acids | | |
| W3xx115-T2U0.E44- | C terminal truncation: | no linker | Fab |
| 10.uIgG4V1 | 8 amino acids | | |
| W3xx115-T2U0.E44- | C terminal truncation: | no linker | Fab |
| 11.uIgG4V1 | 9 amino acids | | |
| W3xx115-T2U0.E44- | F42G substitution | no linker | Fab |
| 12.uIgG4V1 | | | |
| W3xx115-T2U0.E44- | F42V substitution | no linker | Fab |
| 13.uIgG4V1 | | | |
| W3xx115-T2U0.E44- | F42I substitution | no linker | Fab |
| 14.uIgG4V1 | | | |
| W3xx115-T2U0.E44- | C terminal truncation | no linker | Fab |
| 15.uIgG4V1 | of 4 amino acids and | | |
| | F42V substitution | | |
| W3xx115-T2U0.E44- | C terminal truncation | no linker | Fab |
| 16.uIgG4V1 | of 4 amino acids and | | |
| | F42I substitution | | |
| W3xx115-T2U0.E44- | C terminal truncation | no linker | Fab |
| 17.uIgG4V1 | of 5 amino acids and | | |
| | F42V substitution | | |
| W3xx115-T2U0.E44- | C terminal truncation | no linker | Fab |
| 18.uIgG4V1 | of 5 amino acids and | | |
| | F42I substitution | | |
| W3xx115-T2U0.E44- | C terminal truncation | no linker | Fab |
| 19.uIgG4V1 | of 4 amino acids and | | |
| | F42M substitution | | |
| W3xx115-T2U0.E44- | C terminal truncation | no linker | Fab |
| 20.uIgG4V1 | of 4 amino acids and | | |
| | R38W substitution | | |
| W3xx115-T2U0.E44- | C terminal truncation | no linker | Fab |
| 21.uIgG4V322 | of 4 amino acids and | | |
| | T111A substitution | | |
| W3xx115-T2U0.E44- | C terminal truncation | no linker | Fab |
| 22.uIgG4V322 | of 4 amino acids and | | |
| | T111E substitution | | |
| W3xx115-T2U0.E44- | R38W substitution | no linker | Fab |
| 23.uIgG4V1 | | | |
| W3xx115-T2U0.E44- | C terminal truncation | no linker | Fab |
| 24.uIgG4V1 | of 4 amino acids and 1 | | |
| | amino acid deletion in | | |
| | IgG4 N-terminal hinge | | |
| W3xx115-T2U0.E44- | C terminal truncation | no linker | Fab |
| 25.uIgG4V1 | of 4 amino acids and 2 | | |
| | amino acids deletion in | | |
| | IgG4 N-terminal hinge | | |
| W3xx115-T2U0.E44- | C terminal truncation | no linker | Fab |
| 26.uIgG4V1 | of 4 amino acids and | | |
| | I129L substitution | | |
| W3xx115-T2U0.E44- | C terminal truncation | no linker | Fab |
| 27.uIgG4V1 | of 4 amino acids and | | |
| | I129V substitution | | |
| W3xx115-T2U0.E44- | C terminal truncation | no linker | Fab |
| 28.uIgG4V1 | of 4 amino acids and | | |
| | I129A substitution | | |
| W3xx115-T2U0.E44- | C terminal truncation | no linker | Fab |
| 29.uIgG4V1 | of 4 amino acids and | | |
| | I129S substitution | | |
| W3xx115-T2U0.E44- | C terminal truncation | no linker | Fab |
| 30.uIgG4V1 | of 4 amino acids and | | |
| | I129T substitution | | |
| W3xx115-T2U0.E44- | C terminal truncation | no linker | Fab |
| 31.uIgG4V322 | of 4 amino acids and | | |
| | I129L, F42V substitution | | |
| W3xx115-T2U0.E44- | C terminal truncation | no linker | Fab |
| 32.uIgG4V322 | of 4 amino acids and | | |
| | I129L, F42I substitution | | |
| W3xx115-T2U0.E44- | C terminal truncation | no linker | Fab |
| 33.uIgG4V322 | of 4 amino acids and | | |
| | I129L, R38W substitution | | |
| W3xx115-T2U0.E44- | C terminal truncation | no linker | Fab |
| 34.uIgG4V322 | of 4 amino acids and | | |
| | S127A substitution | | |
| W3xx115-T2U0.E44- | C terminal truncation | no linker | Fab |
| 35.uIgG4V322 | of 4 amino acids and | | |
| | S127T substitution | | |
| W3xx115-T2U0.E44- | C terminal truncation | no linker | Fab |
| 36.uIgG4V322 | of 4 amino acids and | | |
| | S127D substitution | | |
| W3xx115-T2U0.E44- | C terminal truncation | no linker | Fab |
| 37.uIgG4V322 | of 4 amino acids and | | |
| | S127N substitution | | |
| W3xx115-T2U0.E44- | C terminal truncation | no linker | Fab |
| 38.uIgG4V322 | of 4 amino acids and | | |
| | C125A substitution | | |
| W3xx115-T2U0.E44- | C terminal truncation | no linker | Fab |
| 39.uIgG4V322 | of 4 amino acids and | | |
| | I128A substitution | | |
| W3xx115-T2U0.E44- | C terminal truncation | no linker | Fab |
| 40.uIgG4V322 | of 4 amino acids and | | |
| | E110R substitution | | |
| W3xx115-T2U0.E44- | C terminal truncation | no linker | Fab |
| 41.uIgG4V322 | of 4 amino acids and | | |
| | E110I substitution | | |
| W3xx115-T2U0.E44- | C terminal truncation | no linker | Fab |
| 42.uIgG4V322 | of 4 amino acids and | | |
| | E110T substitution | | |
| W3xx115-T2U0.E44- | C terminal truncation | no linker | Fab |
| 43.uIgG4V322 | of 4 amino acids and | | |
| | I122Y substitution | | |
| W3xx115-T2U0.E44- | C terminal truncation | no linker | Fab |
| 44.uIgG4V322 | of 4 amino acids and | | |
| | I122T substitution | | |
| W3xx115-T2U0.E44- | C terminal truncation | no linker | Fab |
| 45.uIgG4V322 | of 4 amino acids and | | |
| | I122V substitution | | |
| W3xx115-T2U0.E44- | C terminal truncation | no linker | Fab |
| 46.uIgG4V322 | of 4 amino acids and | | |
| | I28P substitution | | |
| W3xx115-T2U0.E44- | C terminal truncation | no linker | Fab |
| 47.uIgG4V322 | of 4 amino acids and | | |
| | K32D substitution | | |
| W3xx115-T2U0.E44- | C terminal truncation | no linker | Fab |
| 48.uIgG4V322 | of 4 amino acids and | | |
| | E52G substitution | | |
| W3xx115-T2U0.E44- | C terminal truncation | no linker | Fab |
| 49.uIgG4V322 | of 4 amino acids and | | |
| | K76R substitution | | |
| W3xx115-T2U0.E44- | C terminal truncation | no linker | Fab |
| 50.uIgG4V322 | of 4 amino acids and | | |
| | F78G substitution | | |
| W3xx115-T2U0.E44- | C terminal truncation | no linker | Fab |
| 51.uIgG4V322 | of 4 amino acids and | | |
| | P82Y substitution | | |
| W3xx115-T2U0.E44- | C terminal truncation | no linker | Fab |
| 52.uIgG4V322 | of 4 amino acids, | | |
| | E110R and I129L | | |
| | substitution | | |
| W3xx115-T2U0.E44- | C terminal truncation | no linker | Fab |
| 53.uIgG4V322 | of 4 amino acids, | | |
| | E110I and I129L | | |
| | substitution | | |
| W3xx115-T2U0.E44- | C terminal truncation | no linker | Fab |
| 54.uIgG4V322 | of 4 amino acids, | | |
| | E110T and I129L | | |
| | substitution | | |
| W3xx115-T2U0.E44- | C terminal truncation | no linker | Fab |
| 55.uIgG4V322 | of 4 amino acids, | | |
| | I122Y and I129L | | |
| | substitution | | |
| W3xx115-T2U0.E44- | C terminal truncation | no linker | Fab |
| 56.uIgG4V322 | of 4 amino acids, | | |
| | I122T and I129L | | |
| | substitution | | |
| W3xx115-T2U0.E44- | C terminal truncation | no linker | Fab |
| 57.uIgG4V322 | of 4 amino acids, | | |
| | I122V and I129L | | |
| | substitution | | |
| W3xx115-T2U0.E44- | C terminal truncation | no linker | Fab |
| 58.uIgG4V322 | of 4 amino acids, | | |
| | I28P and I129L | | |
| | substitution | | |
| W3xx115-T2U0.E44- | C terminal truncation | no linker | Fab |
| 59.uIgG4V322 | of 4 amino acids, | | |
| | K32D and I129L | | |
| | substitution | | |
| W3xx115-T2U0.E44- | C terminal truncation | no linker | Fab |
| 60.uIgG4V322 | of 4 amino acids, | | |
| | E52G and I129L | | |
| | substitution | | |
| W3xx115-T2U0.E44- | C terminal truncation | no linker | Fab |
| 61.uIgG4V322 | of 4 amino acids, | | |
| | K76R and I129L | | |
| | substitution | | |
| W3xx115-T2U0.E44- | C terminal truncation | no linker | Fab |
| 62.uIgG4V322 | of 4 amino acids, | | |
| | F78G and I129L | | |
| | substitution | | |
| W3xx115-T2U0.E44- | C terminal truncation | no linker | Fab |
| 63.uIgG4V322 | of 4 amino acids, | | |
| | P82Y and I129L | | |
| | substitution | | |
| W3xx115-T2U0.E44- | WT | no linker | Fab |
| 1.uIgG4V322 | | | |
| W3xx115-T3U0.E44- | F42V/Y45A/L72G | no linker | Fab |
| 1.uIgG4V322 | | | |
| W3xx115-T2.Z20- | C terminal truncation: | no linker | None |
| 1.uIgG4V322 | 4 amino acids, bivalent | | |
| W3xx115-T2.Z20- | C terminal truncation | no linker | None |
| 1.uIgG4V322 | of 4 amino acids and | | |
| | I129L substitution, | | |
| | bivalent | | |
| W3xx115-BMK1 | T3A, D20N, N71K, | no linker | Fab |
| | C125S (Xencor) | | |
| W3xx115-BMK7 | Mouse surrogate of | no linker | Fab |
| | BMK8 (SyntheKine) | | |
| W3xx115-BMK8 | L18R, Q22E, Q126K | no linker | Fab |
| | (SyntheKine) | | |
| W3xx115-BMK9 | L19D, C125I, Q126E | no linker | Fab |
| | (Cugene) | | |

### Generation of IL-2 Fc Fusion Proteins in Expi293 Cells

Expi293 cells (Thermofisher, A14635) or ExpiCHO cells (Thermofisher, A29133) were prepared for protein expression, and diluted with pre-warmed Expi293 Expression Medium. The transfection reagents were composed of A and B. Where, the regent A was prepared by adding plasmids into pre-warmed Opti-MEM, and reagent B was prepared by adding transfection reagent to Opti-MEM as well. Then reagent A and B were mixed gently and allowed to incubate for 20 minutes at room temperature. For the transfection procedure, the mixture above was added to cells followed by incubation on shaker in 37° C., 8% CO2, and rotating at 120 rpm for 18-20 hours. After transfection, Enhancer 1 and Enhancer 2 were added to the medium with culture for another 5 days to harvest supernatant.

### Purification of IL-2 Fc Fusion Proteins

The supernatant of Expi293 cells or ExpiCHO cells as described above were collected and filtered for purification using Protein A column (GE Healthcare, Cat. 175438) or Protein G column (GE Healthcare, Cat. 170618). The concentration of purified Fc-fusion proteins was determined by absorbance at 280 nm. The molecular weight and purity were tested by SDS-PAGE and SEC-HPLC, respectively.

The Fc fusion proteins of IL-2 and its variants were generated with purity over 90%, indicating they are intact and well-assembled molecules under physiological condition.

### 1.3 Generation of IL-2Rα, IL-2Rβ and IL-2Rγ

Polynucleotides encoding extracellular domains of IL2Rα (CD25, SEQ ID NO: 135) and IL2Rβ (CD122, SEQ ID NO: 136) and IL2Rγ (CD132, SEQ ID NO: 137), with 6×His tag on C-terminal, were synthesized by Sangon Biotech Inc. The vector with CMV promoter, were then transfected into Expi293 cells. The supernatant of transfected Expi293 cells was collected as described above and filtered for purification using Ni-column (GE Healthcare, Cat. 173712). The concentration of purified His-tagged proteins was determined by absorbance at 280 nm, and molecular weight and purity were tested by SDS-PAGE and SEC-HPLC, respectively.

### Example 2

### In Vitro Characterization

### 2.1 Binding Assays in HH, NK92 and MJ20 Cell Lines (FACS)

Human cutaneous T cell lymphoma HH cells, malignant non-Hodgkin's lymphoma NK92 cells and cutaneous T cell lymphoma MJ20 cells were used for binding assay. They both constitutively express IL-2Rβ (CD122) and π (CD132), and NK92 and MJ20 also expresses IL-2Rα (CD25). HH, NK92 and MJ20 cells were cultured following ATCC guidance, then transferred to a 96-plate for binding analysis. Cells were incubated with IL-2/Fc fusion proteins at the indicated concentrations for 30 min at 4° C. to avoid internalization. Binding was detected by 2^{nd} antibody of PE-anti-human Fc at 4° C. for another 30 min. The median fluorescence intensity (MFI) on HH and NK92 cells was measured using FACS.

As shown in FIG. 2, N-terminal fusion protein (E44 format) showed binding in both NK92 (high affinity receptor) and HH (intermediate affinity receptor) cells, while C-terminal fusion protein (F114) showed lower affinity in NK92 cells, meaning its lower affinity to IL-2Rα.

The IL-2 structure informed that its N terminal was close to C terminal, and they are both on the IL-2Rβ/γc binding interface. Thus, flexibility of IL-2 in a fusion protein with Fc also showed influence on its affinity. As FIG. 3 has shown, the presence of (G4S)n linker in IgG1 affected affinity of IL-2 in a less degree. Similarly, IgG4 with shorter hinge showed weak reduction in binding to high/intermediate affinity receptors, as shown in binding curves in HH and NK92 cells (FIG. 3). Different Fc isotypes in the fusion protein do not significantly affect affinity of IL-2.

TABLE 5
Binding affinity of IL-2/Fc fusion proteins in different formats.

| | EC₅₀ in | EC₅₀ in |
|---|---|---|
| No. | NK92 (nM) | HH (nM) |
| W3XX115-U0T2.F114-1.uIgG1V320 | N.A. | 48 |
| W3XX115-T2U0.E44-2.uIgG1V320 | 4.1 | 33 |
| W3XX115-T2U0.E44-1.uIgG1V320 | 4.9 | 43 |
| W3XX115-T2U0.E44-1.uIgG4V1 | 14 | 48 |

2.2 pSTAT5 Activation Assay of Human CD8+ T Cells

STAT5 is a downstream signal maker strictly associated with T cell activation. Human resting CD8+ T in PBMC were analyzed for STAT5 phosphorylation following 30 minutes' incubation with IL-2 fusion proteins comprising WT IL-2 or IL-2 variants. At the end of the treatment, PBMC were immediately fixed by BD Phosflow Fix Buffer I and then incubated with pre-chilled BD Phosflow Perm Buffer III. After incubation and fixation, cells were stained with anti-CD3, anti-CD4 and anti-CD8 antibodies for 30 minutes at room temperature. After that, cells were treated with Perm Buffer III for permeabilization, and allowed for staining with anti-pSTAT5 antibody for 30 minutes.

Lymphocytes were first gated on the basis of SSC and FSC, and then gated based on CD3 followed by CD4 and CD8 expression to identify CD8+ T cells. Finally, the phosphorylation level of STAT5 in the CD8+ T populations was determined.

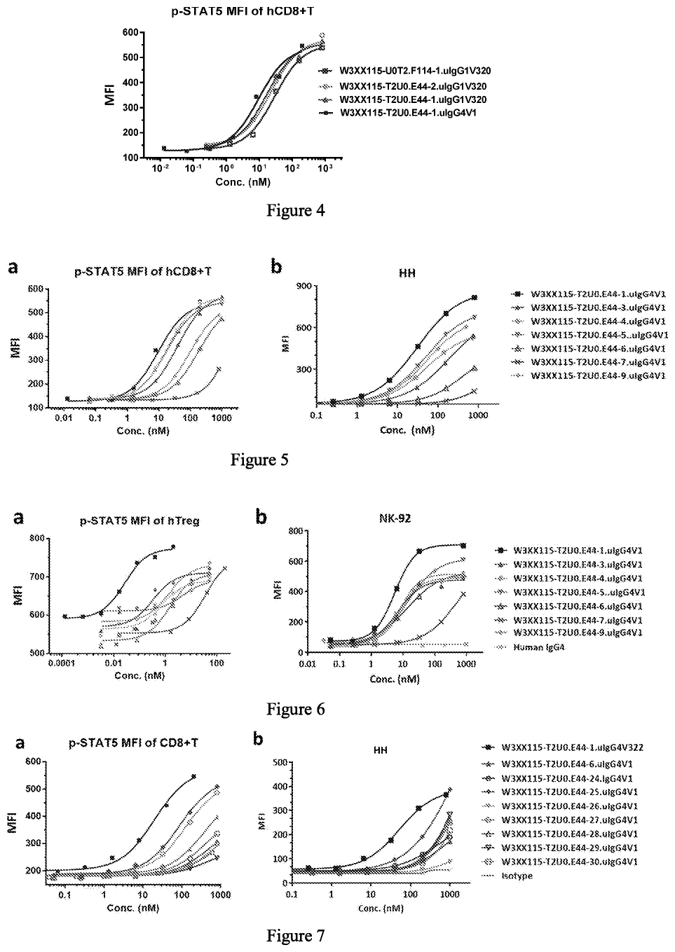

Upon incubation with human CD8+ T cells, the different formats containing a WT IL-2 moiety, showed comparable CD8 activation, as measured by STAT5 phosphorylation (FIG. 4). That is, different formats or flexibility introduced to IL-2 fusion proteins had a little impact on the IL-2Rβ/γc potency of IL-2 (consistent with the result of Table 5).

Primary human CD8+ T (expressing intermediate affinity receptor, IL-2Rβ/γc) were activated by IL-2/Fc fusion proteins comprising IL-2 variants, which is reflected as STAT5 phosphorylation in different degree (FIG. 5a, Table 6). T2U0.E44-7 showed 535-fold potency reduction relative to T2U0.E44-1, followed by T2U0.E44-6 (18-fold) and T2U0.E44-9 (10-fold). Binding curves in HH cells (IL-2Rα/β/γc) demonstrated similar pattern (FIG. 5b).

TABLE 6
Summarized affinity and potency of IL-2/Fc fusion protein comprising truncated IL-2

| | | Human IL-2Rβ/γ | | | | Human IL-2Rα/β/γ complex | | | |
|---|---|---|---|---|---|---|---|---|---|
| | | Potency in | | Affinity | | Potency in | | Affinity | |
| | | human | | in HH | | human | | in NK92 | |
| | | CD8+ T | Potency | EC₅₀ | Affinity | Treg EC₅₀ | Potency | EC₅₀ | Affinity |
| No. | Truncation | EC₅₀ (nM) | reduction | (nM) | reduction | (nM) | reduction | (nM) | reduction |
| W3XX115-T2U0.E44- | / | 9.54 | 1 | 31 | 1 | 0.026 | 1.00 | 5.50 | 1 |
| 1.uIgG4V1 | | | | | | | | | |
| W3XX115-T2U0.E44- | T | 38.1 | 4.00 | 188 | 6.05 | 0.34 | 13.2 | 6.69 | 1.22 |
| 3.uIgG4V1 | | | | | | | | | |
| W3XX115-T2U0.E44- | LT | 18.4 | 1.93 | 55.2 | 1.78 | 0.77 | 29.8 | 7.56 | 1.37 |
| 4.uIgG4V1 | | | | | | | | | |
| W3XX115-T2U0.E44- | TLT | 18.0 | 1.89 | 50.1 | 1.62 | 4.62 | 179 | 14.5 | 2.63 |
| 5.uIgG4V1 | | | | | | | | | |
| W3XX115-T2U0.E44- | STLT | 175 | 18.3 | 437 | 14.1 | 1.51 | 58.4 | 13.7 | 2.48 |
| 6.uIgG4V1 | | | | | | | | | |
| W3XX115-T2U0.E44- | ISTLT | 5109 | 535 | NA | / | 36.4 | 1405 | ~515 | 94 |
| 7.uIgG4V1 | | | | | | | | | |
| W3XX115-T2U0.E44- | SIISTLT | 96.5 | 10.1 | 54.5 | 1.76 | 1.44 | 55.6 | 8.52 | 1.55 |
| 9.uIgG4V1 | | | | | | | | | |

2.3 pSTAT5 Activation Assay of Human Treg Cells

Human Treg from fresh PBMC was isolated by EasySep™ Human CD4+CD127^{low}CD25+ Regulatory T Cell Isolation Kit (Stemcell −18063). Fresh human PBMC was suspended in buffer in a polystyrene round-bottom tube. CD25 Positive Selection Cocktail and CD4+ T Cell Enrichment Cocktail were added in order to positively select CD25+CD4+ T cells. Then, CD127high Depletion Cocktail was used to label CD127^{high} CD25+CD4+ T cells, and unlabeled cells were Treg.

Human Treg Expansion Kit (Miltenyi−130-095-353) was used for Treg expansion. These isolated Treg was mixed with CD3/CD28 beads and suspended with culture medium with 500 IU/ml IL-2.

Human Treg cells (expressing high affinity receptor, IL-2Rα/β/γc) were also analyzed for STAT5 phosphorylation following 30 minutes' incubation with the indicated IL-2 variants. The expanded human Treg cells were operated in the same method as described above for CD8+ T cells, but anti-Foxp3 antibody was used instead of surface CD markers.

The EC50 of pSTAT5 in Treg cells, in T2U0.E44-3, T2U0.E44-4, T2U0.E44-5, T2U0.E44-6, T2U0.E44-7 and T2U0.E44-9 treatment groups, were reduced to varying degrees (FIG. 6a, Table 6), consistent with results in FIG. 5a. Binding curves in NK-92 cells (IL-2Rα/β/γc) also support same potency trend (FIG. 6b). The above results indicate C-terminal truncation leading to IL-2Rβ/γ attenuated IL-2 variants.

### 2.4 Affinity and Potency of IL-2 Variants on IL-2Rγ Interface

Based on C-terminal truncated IL-2 variants, substitutions at C-terminal (including 125, 126, 128 and 129) and hinge truncation were involved to modulate IL-2Rβγ affinity. Primary human CD8+ T and HH cells were used for IL-2Rβ/γ affinity determination, and human Treg and MJ20 cells were used for IL-2Rα/β/γ affinity determination.

As shown in FIG. 7 and Table 7, T2U0.E44-26, T2U0.E44-27, T2U0.E44-28, T2U0.E44-29 and T2U0.E44-30 induced 18 to 32-fold affinity reduction relative to T2U0.E44-1, and T2U0.E44-24 and T2U0.E44-25 induced less attenuation than T2U0.E44-6. Affinity of IL-2 variants to IL-2Rα/β/γ supported the same results (FIG. 8 and Table 7). T2U0.E44-24, T2U0.E44-25 and T2U0.E44-28 showed no obvious attenuation compared to T2U0.E44-6. And T2U0.E44-26, T2U0.E44-27, T2U0.E44-29 and T2U0.E44-30 induced hundreds to thousands affinity reduction in Treg compared to T2U0.E44-1. Binding curves in MJ20 cells (IL-2Rα/β/γ) also achieved similar trend (FIG. 8b). As a result, the method of C-terminal truncation and substitution of 129 was available to achieve IL-2Rβγ attenuated IL-2 variants.

TABLE 7
Summarized affinity and potency of IL-2/Fc fusion protein comprising
IL-2 variants (substitution at position 129 and hinge truncation)

| | | Human IL-2Rβ/γ | | | | Human IL-2Rα/β/γ complex | | | |
|---|---|---|---|---|---|---|---|---|---|
| | | Potency in | | Affinity | | Potency in | | Affinity | |
| | | human | | in HH | | human | | in MJ20 | |
| | | CD8+ T | Potency | EC₅₀ | Affinity | Treg EC₅₀ | Potency | EC₅₀ | Affinity |
| No. | Design | EC₅₀ (nM) | reduction | (nM) | reduction | (nM) | reduction | (nM) | reduction |
| W3XX115-T2U0.E44- | / | 19.99 | 1.0 | 53.77 | 1.0 | 0.0025 | 1.0 | 14.0 | 1 |
| 1.uIgG4V322 | | | | | | | | | |
| W3XX115-T2U0.E44- | Del STLT | 360 | 18.0 | 533.2 | 9.9 | 0.032 | 12.6 | 56.6 | 4.0 |
| 6.uIgG4V1 | | | | | | | | | |
| W3XX115-T2U0.E44- | Del STLT, del | 125.3 | 6.3 | 437.6 | 8.1 | 0.003 | 1.2 | 18.4 | 1.3 |
| 24.uIgG4V1 | 1 aa in N′ | | | | | | | | |
| | hinge | | | | | | | | |
| W3XX115-T2U0.E44- | Del STLT, del | 84.9 | 4.2 | 1279 | 23.8 | 0.022 | 8.8 | 63.2 | 4.5 |
| 25.uIgG4V1 | 2 aa in N′ | | | | | | | | |
| | hinge | | | | | | | | |
| W3XX115-T2U0.E44- | Del STLT, | 649 | 32.5 | 992.9 | 18.5 | 1.16 | 460 | 42.8 | 3.1 |
| 26.uIgG4V1 | I129L | | | | | | | | |
| W3XX115-T2U0.E44- | Del STLT, | 371 | 18.5 | 2745 | 51.1 | 1.85 | 737 | 75.3 | 5.4 |
| 27.uIgG4V1 | I129V | | | | | | | | |
| W3XX115-T2U0.E44- | Del STLT, | 446 | 22.3 | 838.7 | 15.6 | 0.095 | 37.6 | 103 | 7.4 |
| 28.uIgG4V1 | I129A | | | | | | | | |
| W3XX115-T2U0.E44- | Del STLT, | 406 | 20.3 | 1551 | 28.8 | 3.76 | 1494 | 233 | 17 |
| 29.uIgG4V1 | I129S | | | | | | | | |
| W3XX115-T2U0.E44- | Del STLT, | 493 | 24.7 | 3.8E6 | 7.1E+04 | 2.64 | 1047 | 183 | 13 |
| 30.uIgG4V1 | I129T | | | | | | | | |

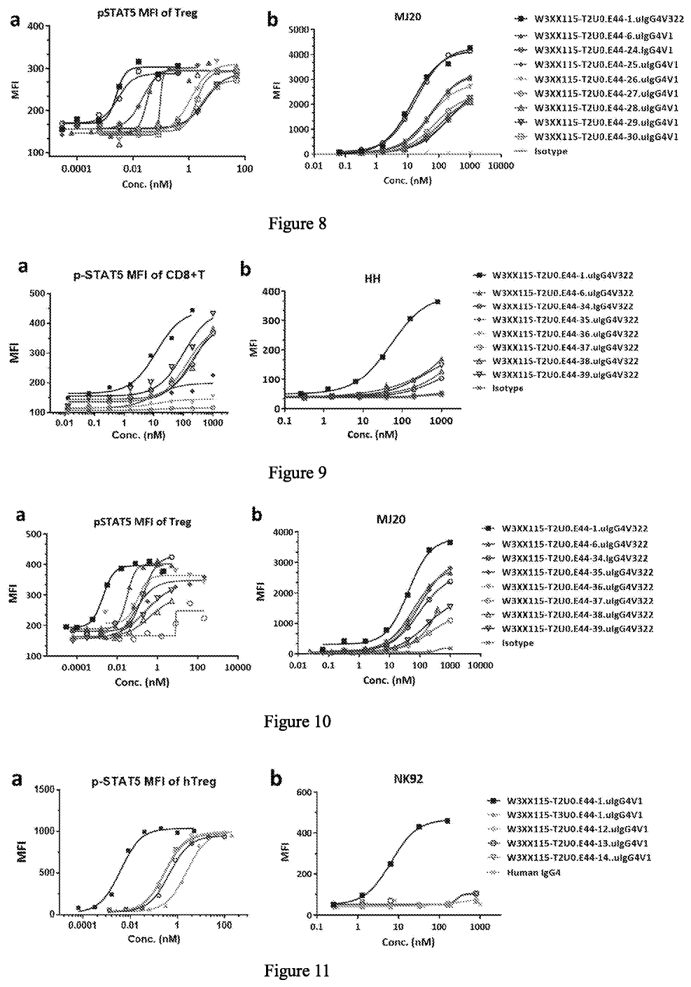

On the basis of truncated IL-2 variant (T2U0.E44-6), substitution of 125, 127 and glycosylation (S127N/I129S) were generated. FIG. 9 and Table 8 showed affinity of these variants to IL-2Rβ/γ, T2U0.E44-34, T2U0.E44-38 and T2U0.E44-39 induced comparable reduction fold to T2U0.E44-6, and T2U0.E44-35, T2U0.E44-36 and T2U0.E44-37 were much weaker in primary CD8+ T. In Treg and MJ20 cells (FIG. 10 and Table 8), T2U0.E44-35 and T2U0.E44-36 showed comparable potency to T2U0.E44-6, and T2U0.E44-37, T2U0.E44-38 and T2U0.E44-39 induced more affinity attenuation.

TABLE 8
Summarized affinity and potency of IL-2/Fc fusion protein comprising IL-2 variants (substitution at positions 125, 127, 129)

| | | Human IL-2Rβ/γ | | | | Human IL-2Rα/β/γ complex | | | |
|---|---|---|---|---|---|---|---|---|---|
| | | Potency in | | Affinity | | Potency in | | Affinity | |
| | | human | | in HH | | human | | in MJ20 | |
| | | CD8+ T | Potency | EC₅₀ | Affinity | Treg EC₅₀ | Potency | EC₅₀ | Affinity |
| No. | Design | EC₅₀ (nM) | reduction | (nM) | reduction | (nM) | reduction | (nM) | reduction |
| W3XX115-T2U0.E44- | / | 10.19 | 1.0 | 52.65 | 1.0 | 0.002 | 1.0 | 41.9 | 1.0 |
| 1.uIgG4V322 | | | | | | | | | |
| W3XX115-T2U0.E44- | Del STLT | 137.7 | 13.5 | 530 | 10 | 0.030 | 15.5 | 101 | 2.4 |
| 6.uIgG4V322 | | | | | | | | | |
| W3XX115-T2U0.E44- | Del STLT, | 180.3 | 17.7 | 719.9 | 13.7 | 0.21 | 104 | 60.9 | 1.5 |
| 34.uIgG4V322 | S127A | | | | | | | | |
| W3XX115-T2U0.E44- | Del STLT, | 10.73 | 1.1 | 576.5 | 10.9 | 0.073 | 37.1 | 92.8 | 2.2 |
| 35.uIgG4V322 | S127T | | | | | | | | |
| W3XX115-T2U0.E44- | Del STLT, | ~12.22 | | 1322 | 25.1 | 0.10 | 52.2 | 98.5 | 2.4 |
| 36.uIgG4V322 | S127D | | | | | | | | |
| W3XX115-T2U0.E44- | Del STLT, | ~17.62 | | 1.0E+07 | 1.9E+05 | 8.0 | 4065 | 87.7 | 2.1 |
| 37.uIgG4V322 | I127N, | | | | | | | | |
| | I129S | | | | | | | | |
| W3XX115-T2U0.E44- | Del STLT, | 268.4 | 26.3 | 2.1E+07 | 4E+05 | 5.0 | 2539 | 201 | 4.8 |
| 38.uIgG4V322 | C125A | | | | | | | | |
| W3XX115-T2U0.E44- | Del STLT, | 99.86 | 9.8 | 355 | 6.8 | 2.0 | 1016 | 74150 | 1770 |
| 39.uIgG4V322 | I128A | | | | | | | | |

2.5 Affinity and potency of IL-2 variants on IL-2Rα interface

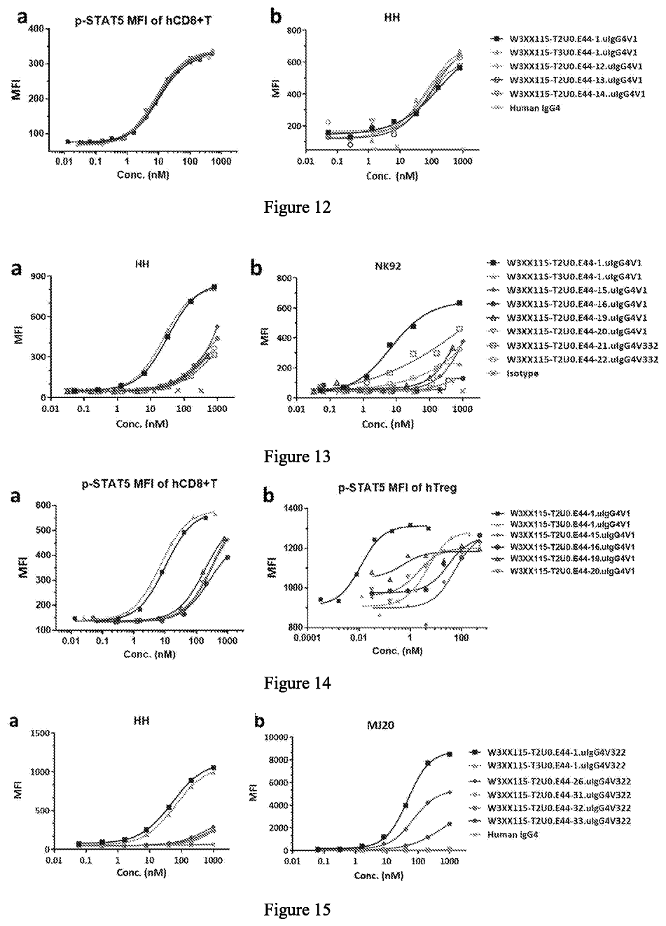

The F42 and R38 amino acids of IL-2 were critical amino acids for IL-2Rα (CD25) binding, and therefore modulated affinity of IL-2 to Treg and active CD8+ T cells. T3U0.E44-1 with F42V/Y45A/L72G mutations in the IL-2 moiety showed 652-fold potency reduction on human Treg (FIG. 11 and Table 9). Other IL-2 mutants on F42, like T2U0.E44-12, T2U0.E44-13, and T2U0.E44-14, all demonstrated dozens to hundreds fold of potency reduction in human Treg (Table 9). However, they all maintained potency to IL-2Rβ/γ complex as T2U0.E44-1 (FIG. 12).

Binding curves in HH and NK92 cells also supported this.

TABLE 9
Summarized affinity and potency of IL-2 mutants.

| | | Human IL-2Rβ/γ | | | | Human IL-2Rα/β/γ complex | | | |
|---|---|---|---|---|---|---|---|---|---|
| | | Potency in | | Affinity | | Potency in | | Affinity | |
| | | human | | in HH | | human | | in NK92 | |
| | | CD8+ T | Potency | EC₅₀ | Affinity | Treg EC₅₀ | Potency | EC₅₀ | Affinity |
| No. | Variants | EC₅₀ (nM) | reduction | (nM) | reduction | (nM) | reduction | (nM) | reduction |
| W3XX115-T2U0.E44- | / | 10.0 | 1 | 74.0 | 1.0 | 0.0038 | 1.00 | 6.98 | 1.00 |
| 1.uIgG4V1 | | | | | | | | | |
| W3XX115-T3U0.E44- | F42A, Y45A, | 7.53 | 0.74 | 72.9 | 1.0 | 2.48 | 652 | NA | / |
| 1.uIgG4V1 | L72G | | | | | | | | |
| W3XX115-T2U0.E44- | F42G | 8.92 | 0.87 | 80.9 | 1.1 | 0.28 | 74.8 | 249 | 35.7 |
| 12.uIgG4V1 | | | | | | | | | |
| W3XX115-T2U0.E44- | F42V | 9.40 | 0.92 | 93.9 | 1.3 | 0.45 | 119 | 245 | 35.1 |
| 13.uIgG4V1 | | | | | | | | | |
| W3XX115-T2U0.E44- | F42I | 8.03 | 0.78 | 82.1 | 1.1 | 0.29 | 76.2 | 238 | 34.1 |
| 14.uIgG4V1 | | | | | | | | | |

2.6 Determination of Potency Reduction of IL-2 Variants with Combo Mutants

C-terminal truncation variant T2U0.E44-6 were combined with singe point mutations of F42, R38 or T111 to generate IL-2 variants. Binding curves to NK92 cells expressing the high affinity TL-2 receptor and HH cells expressing the intermediate affinity receptor in FIG. 13 and Table 10 also demonstrated the attenuation. Accordingly, potency of these variants on human primary CD8+T and Treg cells in inducing STAT5 phosphorylation were both reduced as well, as shown in FIG. 14 and Table 11.

TABLE 10
Binding parameters in HH and NK92 cells

| | | HH | | NK92 | |
|---|---|---|---|---|---|
| | | Affinity | | Affinity | |
| | | in HH | Affinity | in NK92 | Affinity |
| No. | Variants | EC₅₀ (nM) | reduction | EC₅₀ (nM) | reduction |
| W3XX115- | | 25 | 1 | 7.0 | 1 |
| T2U0.E44- | | | | | |
| 1.uIgG4V1 | | | | | |
| W3XX115- | F42A, Y45A, | 24.5 | 1 | 145 | 20.7 |
| T3U0.E44- | L72G | | | | |
| 1.uIgG4V1 | | | | | |
| W3XX115- | F42V, del | ~470 | 18.8 | 2741 | 91.6 |
| T2U0.E44- | STLT | | | | |
| 15.uIgG4V1 | | | | | |
| W3XX115- | F42I, del | 472 | 18.9 | ~310 | 44.3 |
| T2U0.E44- | STLT | | | | |
| 16.uIgG4V1 | | | | | |
| W3XX115- | F42M, del | 398 | 15.9 | 522 | 74.6 |
| T2U0.E44- | STLT | | | | |
| 19.uIgG4V1 | | | | | |
| W3XX115- | R38W, del | 336 | 13.5 | 232 | 33.1 |
| T2U0.E44- | STLT | | | | |
| 20.uIgG4V1 | | | | | |
| W3XX115- | T111A, del | 509 | 20.4 | NA | / |
| T2U0.E44- | STLT | | | | |
| 21.uIgG4V322 | | | | | |
| W3XX115- | T111E, del | 666 | 26.6 | NA | / |
| T2U0.E44- | STLT | | | | |
| 22.uIgG4V322 | | | | | |

TABLE 11
EC₅₀ of STAT5 phosphorylation in human CD8+ T and Treg cells

| | Human CD8+ T | | Human Treg | | |
|---|---|---|---|---|---|
| | Potency | Potency | Potency | Potency | EC₅₀ ratio |
| No. | EC₅₀ (nM) | reduction | EC₅₀ (nM) | reduction | Treg/CD8+ T |
| W3XX115- | 10.3 | 1 | 0.012 | 1 | 858 |
| T2U0.E44- | | | | | |
| 1.uIgG4V1 | | | | | |
| W3XX115- | 7.14 | 0.69 | 3.52 | 296 | 2 |
| T3U0.E44- | | | | | |
| 1.uIgG4V1 | | | | | |
| W3XX115- | 312 | 30.3 | 54.93 | 4616 | 5.7 |
| T2U0.E44- | | | | | |
| 15.uIgG4V1 | | | | | |
| W3XX115- | 251 | 24.4 | 31.22 | 2624 | 8.0 |
| T2U0.E44- | | | | | |
| 16.uIgG4V1 | | | | | |
| W3XX115- | 185 | 18.0 | 0.53 | 44.5 | 349 |
| T2U0.E44- | | | | | |
| 19.uIgG4V1 | | | | | |
| W3XX115- | 323 | 31.4 | 2.06 | 173 | 157 |
| T2U0.E44- | | | | | |
| 20.uIgG4V1 | | | | | |

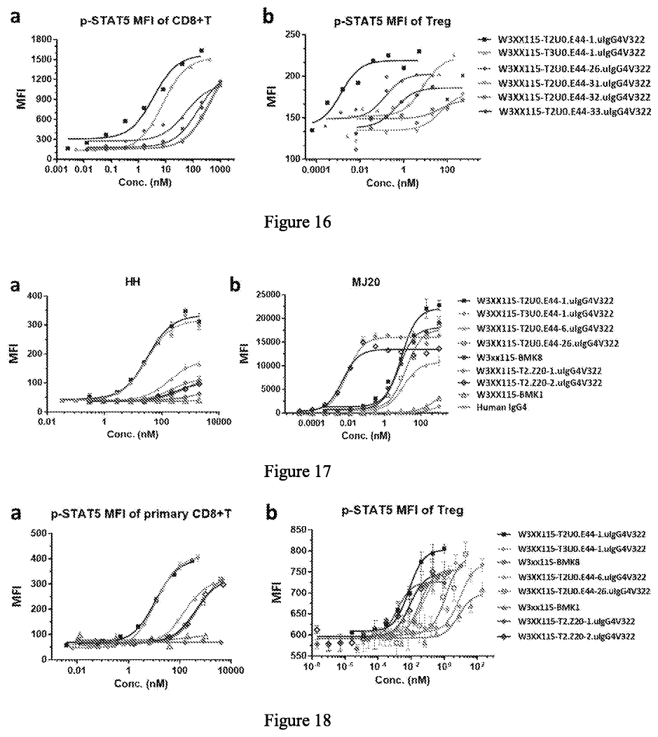

Mutations of F42 or R38 were involved in T2U0.E44-26 to generate IL-2 variants. Binding curves to MJ20 cells demonstrated the attenuation (FIG. 15 and Table 12). Accordingly, potency of these variants on Treg cells were reduced as well, as shown in FIG. 16 and Table 13.

TABLE 12
Binding parameters in HH and MJ20 cells

| | | HH | | MJ20 | |
|---|---|---|---|---|---|
| | | Affinity | | Affinity | |
| | | in HH | Affinity | in MJ20 | Affinity |
| No. | Variants | EC₅₀ (nM) | reduction | EC₅₀ (nM) | reduction |
| W3XX115- | | 50.5 | 1 | 45.9 | 1 |
| T2U0.E44- | | | | | |
| 1.uIgG4V322 | | | | | |
| W3XX115- | F42A, Y45A, | 64.1 | 1.3 | NA | / |
| T3U0.E44- | L72G | | | | |
| 1.uIgG4V322 | | | | | |
| W3XX115- | del STLT, | 626 | 12.4 | 68.9 | 1.5 |
| T2U0.E44- | I129T | | | | |
| 26.uIgG4V322 | | | | | |
| W3XX115- | del STLT, | 800 | 15.8 | NA | / |
| T2U0.E44- | I129T, F42V | | | | |
| 31.uIgG4V322 | | | | | |
| W3XX115- | del STLT, | 904 | 17.9 | NA | / |
| T2U0.E44- | I129T, F42I | | | | |
| 32.uIgG4V322 | | | | | |
| W3XX115- | del STLT, | 760 | 15 | 627 | 13.7 |
| T2U0.E44- | I129T, R38W | | | | |
| 33.uIgG4V322 | | | | | |

TABLE 13
EC₅₀ of STAT5 phosphorylation in human CD8+ T and Treg cells

| | Human CD8+ T | | Human Treg | | |
|---|---|---|---|---|---|
| | Potency | Potency | Potency | Potency | EC₅₀ ratio |
| No. | EC₅₀ (nM) | reduction | EC₅₀ (nM) | reduction | Treg/CD8+ T |
| W3XX115- | 3.65 | 1.0 | 0.0014 | 1.0 | 2670 |
| T2U0.E44- | | | | | |
| 1.uIgG4V322 | | | | | |
| W3XX115- | 7.87 | 2.2 | 7.78 | 5559 | 1 |
| T3U0.E44- | | | | | |
| 1.uIgG4V322 | | | | | |
| W3XX115- | 52.9 | 14.5 | 0.15 | 103.8 | 364 |
| T2U0.E44- | | | | | |
| 26.uIgG4V322 | | | | | |
| W3XX115- | 365.4 | 100 | 48.8 | 34857 | 7 |
| T2U0.E44- | | | | | |
| 31.uIgG4V322 | | | | | |
| W3XX115- | 379 | 104 | 40.9 | 29236 | 17 |
| T2U0.E44- | | | | | |
| 32.uIgG4V322 | | | | | |
| W3XX115- | 149.4 | 41.0 | 0.42 | 302 | 354 |
| T2U0.E44- | | | | | |
| 33.uIgG4V322 | | | | | |

### 2.7 Determination of Potency Reduction of Bivalent IL-2 Variants

IL-2 variants of T2U0.E44-6 and T2U0.E44-26 were used to construct bivalent variants W3XX115-T2.Z20-1.uIgG4V322 and W3XX115-T2.Z20-2.uIgG4V322 respectively. The binding affinity to HH and MJ20 cells were shown in FIG. 17 and Table 14, T2.Z20-1 induced 6.9-fold IL-2Rβ/γ attenuation, similar with T2U0.E44-6 (6-fold). And attenuation fold of T2.Z20-2 was 9.4, more potent than T2U0.E44-26 (20-fold). Affinity differentiation was more significant on IL-2Rα/β/γ cells, bivalent IL-2 variants (EC₅₀, 5 pM and 8 pM) were much more potent than monovalent, and even better than T2U0.E44-1 (EC₅₀, 8.3 nM).

Human primary CD8+ T and Treg cells were used for potency determination of bivalent IL-2 variants (FIG. 18, Table 15). The STAT5 phosphorylation assay results were consistent with binding assay. Reduction folds of bivalent and monovalent IL-2 variants were 13 to 48-fold in primary CD8+ T cells. T2.Z20-1 and T2.Z20-2 induced Treg activation with EC50 of 2 and 8 pM, comparable to T2U0.E44-1 (7 pM). As a result, bivalent TL-2 variants showed over 10,000 potency ratio of Treg/CD8+ T, sharing similar characteristics with BMK8 (∞).

TABLE 14
Binding parameters of bivalent IL-2 variants in HH and MJ20 cells

| | | HH | | MJ20 | |
|---|---|---|---|---|---|
| | | Affinity | | Affinity | |
| | | in HH | Affinity | in MJ20 | Affinity |
| No. | Variants | EC₅₀ (nM) | reduction | EC₅₀ (nM) | reduction |
| W3XX115- | | 31.6 | 1 | 8.31 | 1 |
| T2U0.E44- | | | | | |
| 1.uIgG4V322 | | | | | |
| W3XX115- | F42A, Y45A, | 29.3 | 0.9 | / | / |
| T3U0.E44- | L72G | | | | |
| 1.uIgG4V322 | | | | | |
| W3XX115- | del STLT | 189 | 6.0 | 10.8 | 1.3 |
| T2U0.E44- | | | | | |
| 6.uIgG4V322 | | | | | |
| W3XX115- | del STLT, | 638 | 20 | 15.1 | 1.8 |
| T2U0.E44- | I129T | | | | |
| 26.uIgG4V322 | | | | | |
| W3XX115- | del STLT | 217 | 6.9 | 0.008 | 0.001 |
| T2.Z20- | | | | | |
| 1.uIgG4V322 | | | | | |
| W3XX115- | del STLT, | 297 | 9.4 | 0.005 | 0.001 |
| T2.Z20- | I129T | | | | |
| 2.uIgG4V322 | | | | | |
| W3XX115-BMK1 | T3A, D20N, | / | | ~474 | 57 |
| | N71K, C125S | | | | |
| W3XX115-BMK8 | L19D, C125I, | 2060 | 65 | 5.99 | 0.7 |
| | Q126E | | | | |

TABLE 15
Potency summary of bivalent IL-2 variants in human CD8+ T and Treg cells

| | Human CD8+ T | | Human Treg | | |
|---|---|---|---|---|---|
| | Potency | Potency | Potency | Potency | EC₅₀ ratio |
| No. | EC₅₀ (nM) | reduction | EC₅₀ (nM) | reduction | Treg/CD8+ T |
| W3XX115- | 0.007 | 1.0 | 11.4 | 1 | 1578 |
| T2U0.E44- | | | | | |
| 1.uIgG4V322 | | | | | |
| W3XX115- | 8.99 | 1247 | 11.7 | 1 | 1.3 |
| T3U0.E44- | | | | | |
| 1.uIgG4V322 | | | | | |
| W3XX115- | 0.057 | 7.9 | 148 | 13 | 2591 |
| T2U0.E44- | | | | | |
| 6.uIgG4V322 | | | | | |
| W3XX115- | 1.36 | 188 | 541 | 48 | 398 |
| T2U0.E44- | | | | | |
| 26.uIgG4V322 | | | | | |
| W3XX115- | 0.002 | 0.3 | 532 | 47 | 2.7E+05 |
| T2.Z20- | | | | | |
| 1.uIgG4V322 | | | | | |
| W3XX115- | 0.008 | 1.1 | 410 | 36 | 5.0E+04 |
| T2.Z20- | | | | | |
| 2.uIgG4V322 | | | | | |
| W3XX115-BMK1 | 7.94 | 1101 | / | | ∞ |
| W3XX115-BMK8 | 0.032 | 4.4 | / | | ∞ |

### 2.7 SPR Assay of IL-2 Variants

IL-2 variants binding affinity to IL-2 receptor were detected by SPR using Biacore 8K. For IL-2Rα binding assay, each variant was captured on anti-human Fc immobilized CM5 sensor chip (cytiva). W3XX115-hPro1.ECD.His at different concentrations were injected over the sensor chip at a flow rate of 30 uL/min for an association phase of 60 s, followed by 90 s dissociation. The chip was then regenerated by 10 mM Glycine-HCl pH1.5 after each binding cycle. For IL-2Rγ binding assay, IL-2 variants were mixed with W369-hPro2.ECD.His as 1:1 before assay, were detected by SPR using Biacore 8K. W3XX115-hPro1.ECD.His was captured on anti-His immobilized CM5 sensor chip (cytiva). The mixture were injected over the sensor chip as described above. The sensorgrams of blank surface and buffer channel were subtracted from the test sensorgrams. The experimental data was fitted by steady state affinity model. Molecular weight of W3XX115-hPro1.ECD.His or W369-hPro1.ECD.His was used to calculate the molar concentration of analyte.

As shown in Table 16, T20.E44-1 showed normal affinity to IL-2Rα and IL-2Rγ. The KDs of T2U0.E44-6 and T2U0.E44-26 to IL-2Rα were closed to T20.E44-1, and T2U0.E44-20 and T2U0.E44-33 showed approximate 10-fold affinity reduction compared to T2U0.E44-1. For T3U0.E44-1 and T2U0.E44-33, no binding to IL-2Rα was detected.

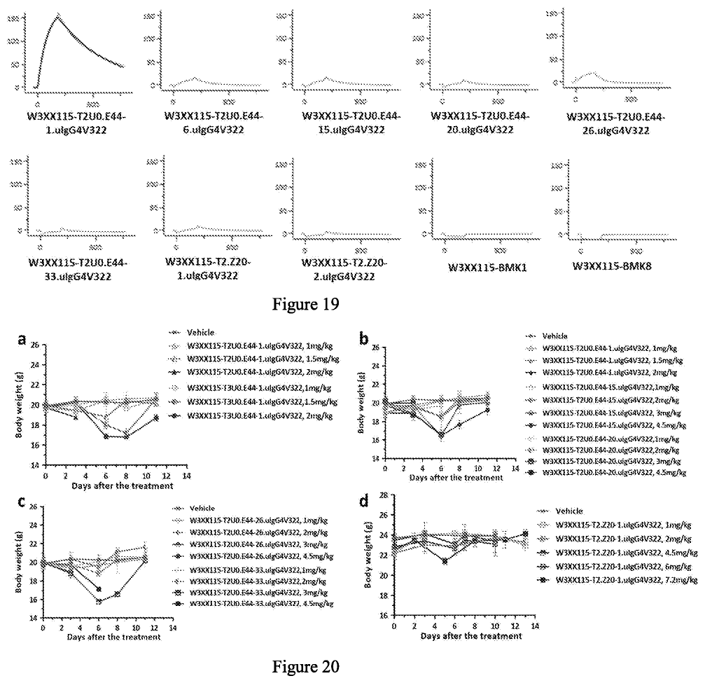

IL-2 variants showed comparable affinity to IL-2Rβ. Affinity of IL-2 variants for IL-2Rγ was too weak to measure directly. IL-2 variants had reduced IL-2Rγ binding relative to T20.E44-1 (FIG. 19), indicating the reduced IL-2Rβ/γ observed in cells is due to impaired interactions with IL-2Rγ.

Bivalent IL-2 variants showed weaker IL-2Rγc binding, and comparable or better IL-2Rα binding to its monovalent parental, but showed decreased affinity to IL-2Rβ. The Fc fusion part of bivalent variants might be leading to steric hindrance between IL-2 molecular, no matter the fusion was on N or C terminal of IL-2, since N or C terminal of IL-2 were both on IL-2Rβ/γc binding interface. Besides Fc moiety, other fusion strategies to form dimers could also contribute to affinity reduction of IL-2 to IL-2Rβ/γc.

TABLE 16
Summarized affinity of IL-2 variants with
IL2Rα, IL-2Rβ and IL2Rγ

| | | KD (M) | | |
|---|---|---|---|---|
| No. | Design | IL-2Rα | IL-2Rβ | IL-2Rγ |
| W3XX115- | | 1.1E−07 | 3.10E−06 | 1.79E−06 |
| T2U0.E44- | | | | |
| 1.uIgG4V322 | | | | |
| W3XX115- | F42A, Y45A, | No binding | 2.26E−06 | Weak |
| T3U0.E44- | L72G | | | binding |
| 1.uIgG4V322 | | | | |
| W3XX115- | del STLT | 9.82E−08 | 1.01E−06 | |
| T2U0.E44- | | | | |
| 6.uIgG4V322 | | | | |
| W3XX115- | del STLT, | No binding | 5.89E−06 | |
| T2U0.E44- | F42V | | | |
| 15.uIgG4V322 | | | | |
| W3XX115- | del STLT, | 1.03E−06 | 6.07E−06 | |
| T2U0.E44- | R38W | | | |
| 20.uIgG4V322 | | | | |
| W3XX115- | del STLT, | 1.07E−07 | 4.27E−05 | |
| T2U0.E44- | I129T | | | |
| 26.uIgG4V322 | | | | |
| W3XX115- | del STLT, | 1.03E−06 | 5.87E−06 | |
| T2U0.E44- | I129T, R38W | | | |
| 33.uIgG4V32 | | | | |
| W3XX115- | del STLT, | 8.99E−08 | 1.02E−04 | |
| T2.Z20- | | | | |
| 1.uIgG4V322 | | | | |
| W3XX115- | del STLT, | 8.16E−08 | 3.70E−04 | |
| T2.Z20- | I129T | | | |
| 2.uIgG4V322 | | | | |

### Example 3

### In Vivo Characterization

All the procedures related to animal handling, care and the treatment in the study were performed according to the guidelines approved by the Institutional Animal Care and Use Committee (IACUC) of LARC of WuXi Biologics following the guidance of the Association for Assessment and Accreditation of Laboratory Animal Care (AAALAC).

### 3.1 Mouse MTD of IL-2 Variants

C57BL/6N mice were used for maximum tolerance dose (MTD) study. Mice were randomly divided into different groups with 3 mice in each group. Mice received test articles in different dosages every 3 days. Then body weight measurement was performed twice a week and mouse observation was performed daily.

As shown in FIG. 20, MTD of attenuated IL-2 variants were all improved in different range compared to T2U0.E44-1 (1.5 mg/kg). MTD of T2U0.E44-6, T2U0.E44-20, T2U0.E44-2, T2U0.E44-33 were 2 mg/kg, and MTD of T2U0.E44-15 was 3 mg/kg. Besides, bivalent IL-2 variants show obvious MTD improvement with over 7.2 mg/kg.

### 3.2 Anti-Tumor Efficacy of IL-2 Variants

MC38 syngeneic model were used for efficacy study. The MC38 cells were implanted (s.c.) into the right flanks of C57BL/6N mice. When tumors reach about 60-80 mm³ in volume, tumor bearing mice were randomly divided into different groups, dosing with IL-2 variants at day 0 and day 3. Tumor sizes were measured in two dimensions using a caliper, and the volume was expressed in mm³. Results were represented by mean and the standard error (Mean±SEM).

Statistical analysis was operated by two-way ANOVA, P<0.05 was considered to be statistically significant.

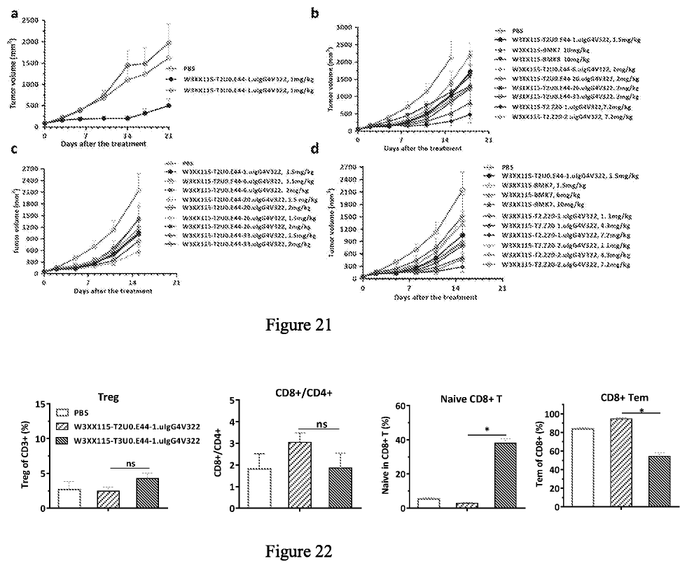

Firstly, non-α IL-2 (W3XX115-T3U0.E44-1.uIgG4V322, comprising an IL-2 variant that does not bind to IL-2Rα) was used to compare with wild-type IL-2 (W3XX115-T2U0.E44-1.uIgG4V322) in MC38 syngeneic model. As shown in FIG. 21a, no efficacy observed after T3U0.E44-1 treatment, and T2U0.E44-1 treatment showed significant anti-MC38 tumor effect, which was consistent with in vitro potency in immune cells (FIG. 18). This demonstrated that IL-2Rα binding is necessary for IL-2 variants, non-Treg affinity means non-activated CD8+ T preference.

IL-2 variants in different doses were tested further in MC38 syngeneic model, compared to wild-type IL-2 and BMK7 (surrogate of BMK8). Treatments of T2U0.E44-6, T2U0.E44-20, T2U0.E44-26 and T2U0.E44-33 at MTD dose (2 mg/kg) showed significant anti-tumor effect comparing to the treatment of PBS (FIG. 21b), with TGI of 34%, 53%, 54% and 62% respectively. There was no significant difference among T2U0.E44-6/20/26/33 and T2U0.E44-1 (TGI=52%, 1.5 mg/kg). For bivalent IL-2 variants, T2.Z20-2 in high dose (7.2 mg/kg, equivalent to 10 mg/kg of BMK7) showed comparable tumor inhibition to above IL-2 variants, with an TGI of 59%. Efficacy of T2.Z20-1 (TGI=88.7%) and BMK7 (TGI=77.7%) were much better than T2U0.E44-1, with an average tumor volume of 478 and 518 mm3 at day 15. Treatment of T2.Z20-1 induced 50% tumor-free (3 tumor-free in 6 mice), and the ratio of BMK7 and T2U0.E44-33 were 33% (2 tumor-free in 6 mice), for T2.Z20-2 was 16.7% (1 tumor-free in 6 mice).

Dose response study of T2U0.E44-6, T2U0.E44-20, T2U0.E44-26 and T2U0.E44-33 were shown in FIG. 21c, doing of 1.5 mg/kg or 2 mg/kg both induced significant anti-tumor effect and similar TGI. Bivalent IL-2 variants and BMK7 showed obvious dose response at 1.1, 4.3, 7.2 mg/kg or equivalent dose (FIG. 21d). The TGIs of T2.Z20-1 in different doses were 29.6, 64.4 and 88.7% from low to high, and TGIs of BMK7 in equivalent doses were 38.9, 79.8, 77.7% respectively.

### 3.3 Pharmdynamic Effect of IL-2 Variants

MC38 syngeneic model were used for PD study of IL-2 variants. Tumor bearing mice were randomly divided into different groups, dosing on day 0 and 3. All mice were euthanized on day 5, then tumors and spleens from these mice were collected for analysis.

As shown in FIG. 22, treatment of non-α IL-2 (W3XX115-T3U0.E44-1.uIgG4V322) also expanded tumor-infiltrating Tregs, did not elevate CD8+/CD4+ ratio in TILs. W3XX115-T2U0.E44-1.uIgG4V322 showed no Treg preference in TIL, but expended more effect memory subtype of CD8+ T (Tem) to support tumor inhibition, suggesting the necessity of IL-2Rα binding for anti-tumor IL-2 variants.

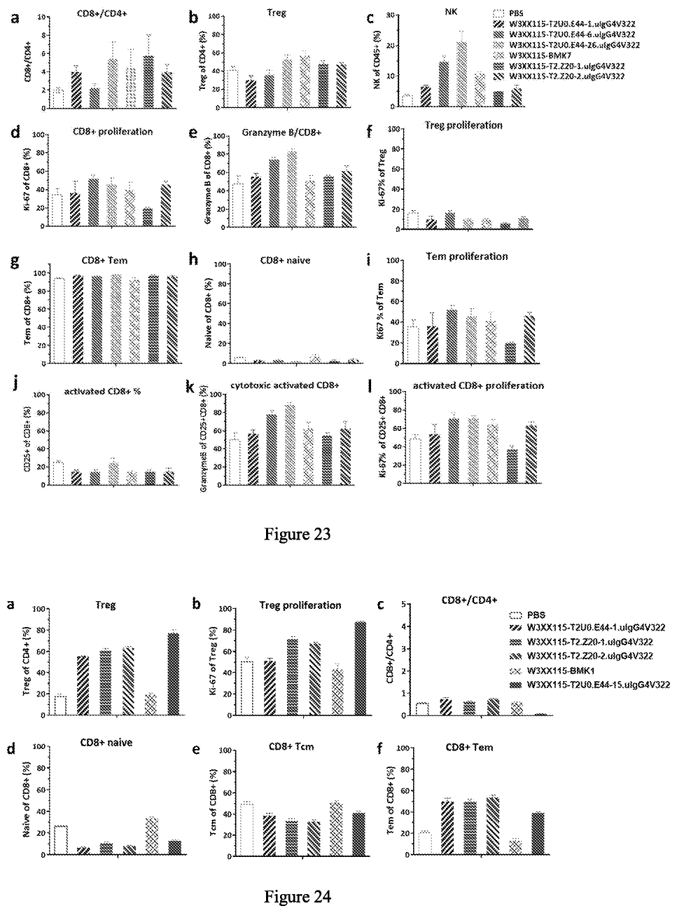

FIG. 23 depicts the immune cell response in tumor post IL-2 variants treatment. IL-2 and variants elevated CD8+/CD4+ ratio in TIL compared to PBS treatment (FIG. 23a), and did not prefer to expend Treg (FIG. 23b, f). T2U0.E44-6 and T2U0.E44-26 induced NK cells infiltration and proliferation, similar to BMK7 (FIG. 23c). In total CD8+ T cells, Ki-67 (proliferation, FIG. 23d) and Granzyme B (cytotoxicity, FIG. 23e) markers were not significant upregulated, except for T2U0.E44-6 and T2U0.E44-26. CD8+ T cells were further divided into naïve and effector memory subtypes by CD44 and CD62L. IL-2 variants expanded more Tem rather than naïve subtypes (FIG. 23g, h), and function of IL-2 variants were slightly better than BMK7 in Tem percentage and proliferation (FIG. 23i). That means these IL-2 variants keep function and biology of IL-2, but with better efficacy and less toxicity.

In TIL, activated CD8+ T, expression of high affinity IL-2 receptors (αβγ), were more sensitive to IL-2. The activated CD8+ T percentages upon different IL-2 variants treatments were comparable (FIG. 23j). IL-2 variants induced more proliferation of activated CD8+ T (FIG. 23l) than wild-type (T2U0.E44-1). Besides, FIG. 24k showed stronger cytotoxicity of activated CD8+ T in T2U0.E44-6 and T2U0.E44-26 treatment groups than others, suggesting that T2U0.E44-6 and T2U0.E44-26 worked in both IL-2Rαβγ cells (activated CD8+ T) and IL-2Rβ7 cells (NK). Bivalent IL-2 variants, T2.Z20-1, expanded more Tem and less Treg in TIL (FIG. 23b, g), dominating better anti-tumor effect.

FIG. 24 describes the peripheral immune cell response post IL-2 variants treatment. T2U0.E44-15 induced more peripheral Treg proliferation, better than T2.Z20-1, T2.Z20-2, T2U0.E44-1 and BMK1 (FIG. 24 a, b). All IL-2 variants elevated neither peripheral CD8+/CD4+ ratio, nor Tem or Tem percentage (FIG. 24d, e, f), suggesting in vivo safety. As a result, T2.Z20-1, T2.Z20-2 and T2U0.E44-15 showed potential to modulate peripheral immune suppression, available for autoimmune disease therapy.

### 3.4 Efficiency of IL-2 Variants in Autoimmune Disease

Colitis is an intestinal bowel disease, a kind of autoimmune disease. Here we used DSS-induced colitis model to explore efficacy of IL-2 variants in autoimmune disease. C57BL/6N mice in 8 weeks were randomly divided into different groups by body weight. To induced colitis, 3% DSS were fed to mice for 6 days. Mice were treated of IL-2 variants at day 1, 4, 7 after grouping. Then body weight and disease activity index (DAI) were collected daily.

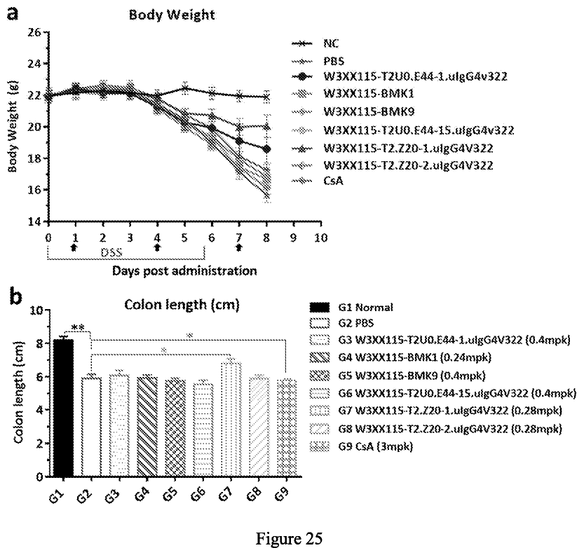

FIG. 25 showed body weight change of colitis mice after IL-2 variants treatment, all variants could alleviated body weight loss caused by colitis compared to non-treatment group. T2.Z20-1 showed better effect than wild-type IL-2 (T2U0.E44-1) in body weight changing (a) and colon length (b). As a result, IL-2 variants showed potential application to autoimmune diseases therapy.

Those skilled in the art will further appreciate that the present invention may be embodied in other specific forms without departing from the spirit or central attributes thereof. In that the foregoing description of the present disclosure provides only exemplary embodiments thereof, it is to be understood that other variations are contemplated as being within the scope of the present invention. Accordingly, the present invention is not limited to the particular embodiments that have been described in detail herein. Rather, reference should be made to the appended claims as indicative of the scope and content of the invention.

1. An interleukin-2 (IL-2) variant, wherein the IL-2 variant has a reduced binding affinity to at least one of IL-2Rα, IL-2Rβ and common γ chain, and has an amino acid sequence comprising mutation(s) selected from the following compared to the amino acid sequence as set forth in SEQ ID NO: 1: a C-terminal truncation and one or more substitutions, wherein the C-terminal truncation is selected from a truncation of 1 to 20 amino acids, wherein the substitution is selected from a substitution at position 42, a substitution at position 28, a substitution at position 32, a substitution at position 38, a substitution at position 42, a substitution at position 52, a substitution at position 76, a substitution at position 78, a substitution at position 82, a substitution at position 110, a substitution at position 111, a substitution at position 122, a substitution at position 125, a substitution at position 127, a substitution at position 128, a substitution at position 129 and any combinations thereof.

2-5. (canceled)

6. The IL-2 variant of claim 1, wherein the amino acid sequence of the IL-2 variant is selected from those as set forth in SEQ ID NOs: 5, 2-4, 6-60 and homologous sequences thereof with at least 95% identity.

7-8. (canceled)

9. The IL-2 variant of claim 1, wherein the binding affinity to at least one of IL-2Rα, IL-2Rβ and common γ chain is measured by SPR or by FACS binding analysis.

10. A fusion protein, comprising the IL-2 variant of claim 1 operably linked to a non-TL-2 moiety.

11. The fusion protein of claim 10, wherein the non-IL-2 moiety is selected from PEGs, lipids, Fc region, human serum albumin (HSA) and anti-HSA moiety, such as an Fc region.

12. (canceled)

13. The fusion protein of claim 10, comprising two amino acid chains, wherein:
(a) each chain comprises one IL-2 variant fused to one chain of the Fc region at the N-terminal of the Fc region;
(b) each chain comprises one IL-2 variant fused to one chain of the Fc region at the C-terminal of the Fc region;
(c) one chain comprises one IL-2 variant fused to one chain of the Fc region at the N-terminal of the Fc region, and the other chain comprises one IL-2 variant fused to the other chain of the Fc region at the C-terminal of the Fc region; or
(d) each chain comprises two IL-2 variants fused to the N-terminal and C-terminal of one chain of the Fc region, respectively.

14-15. (canceled)

16. A polypeptide complex, comprising an IL-2 moiety, an antigen-binding moiety and an Fc region, wherein:
the IL-2 moiety comprises the IL-2 variant of claim 1,
the antigen-binding moiety is selected from a Fab, a VHH, a scFv and a TCR.

17. The polypeptide complex of claim 16, comprising two heavy chains and one or two light chains, wherein the antigen-binding moiety is a Fab, and wherein:
(A) from N-terminal to C-terminal, the first heavy chain comprises the IL-2 moiety and one chain of the Fc region, the second heavy chain comprises a heavy chain of the Fab and the other chain of the Fc region, and the light chain comprises a light chain of the Fab:
(B) from N-terminal to C-terminal, the first heavy chain comprises a heavy chain of the Fab, one chain of the Fc region and the IL-2 moiety, the second heavy chain comprises a heavy chain of the Fab and the other chain of the Fc region, and two light chains each comprising a light chain of the Fab; or
(C) from N-terminal to C-terminal or from C-terminal to N-terminal, the two heavy chains each comprising a heavy chain of the Fab, one chain of the Fc region and one IL-2 moiety, and the two light chains each comprising a light chain of the Fab.

18. The polypeptide complex of claim 16, comprising two chains, wherein the antigen-binding moiety is a VHH or scFv, and wherein:
(A) from N-terminal to C-terminal, the first chain comprises the IL-2 moiety and one chain of the Fc region, the second chain comprises the VHH and the other chain of the Fc region;
(B) from N-terminal to C-terminal, the first chain comprises a VHH, one chain of the Fc region and the IL-2 moiety, the second chain comprises a VHH and the other chain of the Fc region; or
(C) from N-terminal to C-terminal or from C-terminal to N-terminal, each chain comprises the VHH or scFv, one chain of the Fc region; and one IL-2 moiety.

19-22. (canceled)

23. The polypeptide complex of claim 16, wherein the antigen-binding moiety specifically binds to a target antigen selected from PD-1, PD-L1, PD-L2, CTLA-4, LAG3, TIM-3, TIM4, 4-1BB, OX-40, OX-40L, GITR, A2aR, TIGIT, CD96, PVRIG, CD226, 5T4, VISTA, VSIG3, VSIG4, ICOS, CD28, CD3, CD4, CD8, CD45, CD44v6, CD27, CD47, SIRPAa, SLAMF7, CD24, Siglec10, Siglec15, Siglec8, VSIR, VSIG4, PSGL-1, C5AR1, BTN1A1, BTN3A1, CD70, RANKL, CSF1R, CSF2RB, TNFRSF1/1a/1b, BDCA2, BTLA, C5aR, NKG2A, NKG2D, NKp30, NKp46, CD16a, CD56, CD166, FCGR3, CD2, Neurophilin-1, CCR8, CCR2, CCR4, CCR5, CCR6, CCR7, CCR8, GCGR, CXCR2, CXCR4, CXCR5, CALCRL, ETAR, GLP1R, CX3CR1, GPR1, GPR17, GPR20, GPR30, GPR34, GPR-65, GPCR78, GPRC5D, GPR84, LGR4, LGR5, VEGF, VEGFR, HER2, HER3, Trop2, pCAD, ERα, EGFR, de2-7 EGFR, EGFRvIII, PSMA, PSCA, PSA, TAG-72, SEZ6, SEZ6L, SEZ6L2, SEMA4D, DLL3, GD2, GPC3, KLB, KLRB1, KLRG1, GPC1, PCSK9, EpCAM, p-Cadherin, Caludin 6, Caludin 18.2, FGFR2b, FGFR3, FGFR4, MUC1, MUC13, MUC16, MUC17, MUCL3, FolRa, TfR, TF, TFR, TFPI, c-Met, NY-ESO-1, GUCY2C, LIV-1, Integrin αvβ6, Integrin α10β1, Intergrin α3, Integrin α5β4, Integrin αvβ3, Integrin αvβ8, ROR1, ROR2, PRLR, PTK7, B7-H3, Nectin-4, NetG1, Ax1, CD147, LRRC15, Napi2b, STEAP1, LY6G6D, LYPD1, MACRO, MerTK, MICA, MICB, MSLN, Mkars, G12D, CDH3, CDH6, CDH17, APLA2, CAIX, CD46, CD47, CLDN6, EphA3, Fucosyl-GM1, ITGA3, Kallikrein, MISRII, Podocalyxin, RON, ROBO1, PAUF, PLA2, Podocalyxin, PRLR, PTK7, TM4SF1, TMEFF2, TREAKR, TREM-1, TREM-2, uPARAP, TYRP1, KAAG1, RU2AS, CD146, CD63, Endoglin, Globo H, IGF-1R, TEM1, TEM8, TAX1BP3, ADAM-9, ENPP3, EphA2, EphA3, FcRH5, NaPi3b, TWEAK, DLK1, SORT1, SSTR2, STEAP1, CD25, CD39, GARP, LRRC33, LAIR1, LAMP3, LAP, LEPR, LILRB1, LILRB2, LILRB4, RAGE, FGL1, TPBG, PDGFRB, TGFBR2, CEACAM1, CEACAM5, CEACAM6, Carcinoembryonic antigen (CEA), ICAM1, A33, CAMPATH-1 (CDw52), Carboanhydrase IX (MN/CA IX), CD248, PDPN, ITGB1, ITGAV, CD20, CD19, CD21, CD22, CLL, BCMA, DCLK1, DDR1, DLK1, DPEP3, DKK1, CD5, CD13, CD30, CD33, CD34, CD36, CD37, CD38, CD43, CD52, CD55, CD94, CD99, CD7, CD71, CD73, CD74, CD79a, CD79b, CD229, CD132, CD133, G250, CSF1R (CD115), HLA-DR, HLA-G, HTRA1, TRA-1-60, IGFR, IL-2 receptor, MCSP (Melanoma-associated cell surface chondroitin sulphate proteoglycan), ART1, ASGR1, B7H3, B7-H4, B7H6, CD124, c-Kit (CD117), CD7, Clex12A, Clever-1, IL-13RA2, IL-11RA, IL-31RA, IL-4RA, IFNAR, ActRIIb, IL-7R, SLAMF7, Fms-like tyrosine kinase 3 (FLT-3, CD135), GFRA1, BTLA, GloboH, CSF2RB, chondroitin sulfate proteoglycan 4 (CSPG4), ITGA4, Clec5a, Clec7a, Clec9a, Clec12a, CLEC14, CD205, CD206, CD200R1, CD228, CD229, CD40, CD40L, FcRn, TLR8, TLR9, TNFR2, LTBR, CD44, CD93, PDGF, PDGFR-alpha (CD140a), PDGFR-beta (CD140b), CD146, CD147, CRTH2, TNF-α, TGF-β, IL1RAcP, TSLP, DR5, ST2, fibroblast activating protein (FAP), CDCP1, Derlin1, Tenascin, frizzled 1-10, the vascular antigens VEGFR2 (KDR/FLK1), VEGFR3 (FLT4, CD309), Endoglin, Tie2 and other TAAs, I/O checkpoints, tumor microenvironment targets, or autoimmune and inflammatory diseases associated targets.

24. The polypeptide complex of claim 16, comprising a hinge region between the TL-2 moiety and the Fc region and/or between the antigen-binding moiety and the Fc region, wherein optionally the hinge region has a truncation of 1 to 10 amino acids from the N-terminal or C-terminal compared to an immunoglobulin hinge region.

25. (canceled)

26. The polypeptide complex of claim 16, comprising a linker between the IL-2 moiety and the Fc region and/or between the antigen-binding moiety and the Fc region.

27-31. (canceled)

32. The polypeptide complex of claim 16, comprising:
(a) a first heavy chain that comprises an amino acid sequence selected from those set forth in SEQ ID NOs: 62-129 and homologous sequences thereof with at least 95% identity;
(b) a second heavy chain that comprises an amino acid sequence selected from those set forth in SEQ ID NOs: 131-133 and homologous sequences thereof with at least 95% identity; and
(c) a light chain that comprises an amino acid sequence selected from those set forth in SEQ ID NO: 134 and homologous sequences thereof with at least 95% identity.

33. An isolated nucleic acid molecule, comprising a nucleic acid sequence encoding the IL-2 variant of claim 1.

34. A vector or host cell comprising the nucleic acid molecule of claim 33.

35. (canceled)

36. A pharmaceutical composition comprising the polypeptide complex of claim 16, and a pharmaceutically acceptable carrier.

37. An immunoconjugate comprising the polypeptide complex of claim 16 conjugated to an agent, such as chemotherapeutic agent, radioactive particle or toxin.

38. A method for producing the polypeptide complex of claim 16 comprising the steps of:
expressing the polypeptide complex in the host cell comprising a vector(s) encoding the heavy chain and/or light chain of the polypeptide complex; and
isolating the polypeptide complex from the host cell culture.

39. (canceled)

40. A method for treating or preventing a cancer, an autoimmune disease, or an inflammatory disease in a subject, comprising administering an effective amount of the polypeptide complex of claim 16 to the subject.

41. The method of claim 40, which further comprises administering an additional anti-tumor therapy, such as cell immunotherapy including tumor-infiltrating lymphocyte (TIL) therapy, T cell receptor (TCR) therapy, chimeric antigen receptor (CAR) T cell therapy, macrophage cell therapy, and NK cell therapy, targeted therapy, chemotherapy and gene therapy.

42. The method of claim 40, wherein the cancer is selected from breast cancer, lung cancer, colon cancer, ovarian cancer, melanoma, bladder cancer, renal cell carcinoma, liver cancer, prostate cancer, stomach cancer, pancreatic cancer, NSCLC, non-Hodgkin's lymphoma, chronic lymphocytic leukemia, diffuse large B-cell lymphoma, and multiple myeloma, and/or the autoimmune disease or the inflammatory disease is selected from inflammatory bowel disease, multiple sclerosis, rheumatoid arthritis, systemic lupus erythematosus, aplastic anemia, coeliac disease, type1 diabetes, graves' disease, psoriasis, and scleroderma.

43-46. (canceled)

47. A kit comprising a container comprising the polypeptide complex of claim 16.
